(19) United States
(12) Patent Application Publication
(10) Pub. No.: US 2024/0053343 A1
(43) Pub. Date: Feb. 15, 2024

(54) BIOMARKERS SIGNATURE(S) FOR THE PREVENTION AND EARLY DETECTION OF GASTRIC CANCER

(71) Applicants: INSTITUT PASTEUR, PARIS (FR); ASSISTANCE PUBLIQUE - HOPITAUX DE PARIS, PARIS (FR); UNIVERSITE DE VERSAILLES SAINT QUENTIN EN YVELINES, VERSAILLES (FR)

(72) Inventors: Eliette Camille TOUATI, PARIS (FR); Valerie MICHEL, PARIS (FR); Dominique LAMARQUE, PARIS (FR); Quentin GIAI GIANETTO, PARIS (FR); Thibault DOUCHE, PARIS (FR); Mariette MATONDO, PARIS (FR)

(21) Appl. No.: 18/258,271

(22) PCT Filed: Dec. 21, 2021
(86) PCT No.: PCT/EP2021/087155

§ 371 Date: Jun. 19, 2023

(30) Foreign Application Priority Data
Dec. 21, 2020 (EP) 20306649.3

(51) Int. Cl. G01N 33/574; G16H 50/20

(52) U.S. Cl. CPC G01N 33/57484; G01N 33/57446; G16H 50/20; G01N 2800/50

(57) ABSTRACT

The invention relates to an in vitro method of determining whether a human patient has lesions rendering said patient at risk of a gastric cancer condition and/or needs further medical test in relation thereto, comprising screening a biological sample of blood or plasma, where the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1 (SAA2), LBP, DSP, KRT2, KRT14, ARG1, S100A12, ATAD3B, MAN1A1, HAL, DCD, 07, HP, LEP, IL-8, IL-17, TNF-alpha, USF1, USF2, SELE, MSLN, EGFR, STATS and mtDNA level is determined, with the proviso that the selected biomarkers do not consist of the association of IL-8 and mtDNA level. The method may be for assessing the risk that a human patient has a non-atrophic gastritis (NAG), or an atrophic gastritis/pre-neoplasia (AG/P), or a gastric cancer (GO), or for discriminating between those risks or presence of these conditions. In a particular aspect, the method may comprise as a distinct, simultaneous or parallel step, a step of detecting an Helicobacter pylori infection. The invention also relates to a kit suitable for carrying out such a method, or a set of markers, and the use of the same for determining whether a human patient has lesions rendering said patient at risk of a gastric cancer condition and/or needs further medical test in relation thereto, or for prognosing or diagnosing a gastric pre-cancer condition or a gastric cancer condition.

### FIELD OF THE INVENTION

The invention relates to the field of in vitro testing methods based on the investigation of, preferably, plasmatic biomarkers obtained from biological samples collected from individuals, especially humans, and in particular relates to methods that can be applied to the prognosis or diagnosis process of a gastric cancer condition or gastric pre-cancer condition, or to the monitoring of a patient susceptible of suffering from the same. By gastric cancer condition or gastric pre-cancer condition, it is meant gastric cancer or condition(s) that may evolve in a gastric cancer, i.e., condition(s) that may precede a formal gastric cancer outcome, according to different stages, from asymptomatic to symptomatic ones. The invention takes place in a context where early detection of gastric cancer is sought. Accordingly, the invention also relates to kits, sets of markers for performing the methods of the invention, and their uses. Use of the biomarkers described herein may allow to determine whether further clinical investigations should be carried out, or evaluate a risk, or to ultimately diagnose a risk or a disease, and to favor the cure of a disease that needs to be diagnosed precociously in order to escape a poor prognosis.

### BACKGROUND OF THE INVENTION

### INTRODUCTION

Gastric cancer (GC) still represents a major public health problem with about 1 million deaths per year worldwide. GC remains the third cause of cancer-related death and the fourth most diagnosed cancer (1). While the incidence of GC was observed to decline during the last 30 years worldwide, the number of new cases is set to remain at the best constant or to increase up to 2030, due to population growth and aging. Recently an increase of new and unusual GC cases has been reported in people under 50, mainly males, in both low- and high-income countries (2). These data lead to predict a growing incidence of GC, highlighting that this cancer remains an important challenge for public health on a global scale. GC is mostly associated with a poor prognosis with an overall 5-years survival rate of 15%, thus highlighting the importance of its early detection. GC results from a multistep process starting by the development of a chronic inflammation that evolves through pre-neoplasia (intestinal metaplasia and dysplasia) to cancer lesions (3). The major risk factor responsible for 90% of GC cases is Helicobacter pylori infection which affects half of the world population.

While gastric cancer carries a poor prognosis when diagnosed at an advanced stage, it can be a curable disease if it is diagnosed at an early stage. Nonetheless, GC is often asymptomatic or causes only nonspecific symptoms in its early stages. By the time heavy symptoms occur, the cancer has often reached an advanced stage and may have also metastasized. Thus, there is still a need for characterization and validation of early GC biomarkers to reduce the morbidity and mortality associated to gastric adenocarcinoma.

Importantly, GC can be prevented if a pre-cancer condition is detected at an early stage, at the best before the development of pre-neoplasia (4). The eradication of H. pylori infection has been proposed to prevent GC. However, it is not sufficient as the magnitude of risk reduction depends of the timing of eradication during the pre-neoplastic process (5). Presently, GC can be only diagnosed by gastric endoscopy usually performed under general anesthesia. Importantly, it should be observed that while this technique involving a biopsy comes with an accurate diagnosis when GC is present, neoplastic lesions cannot always be detected with this invasive technique, since in case of neoplastic lesions, the recovered sample to be assayed may be drawn from a region of the gastric tract where no lesion is present. This technique is also very invasive, requiring general anaesthesia.

Furthermore and unfortunately, no appropriate screening strategies are available for large-scale application to reduce the global burden of GC diagnosis. The development of non-invasive methods as blood-based biomarkers is crucial as a powerful contribution for diagnostic tools, not only for the early detection and prevention of patients at risk of GC but also to predict disease recurrence/outcome and to monitor anticancer therapy, hence for improving the survival of GC patients.

The inventors identified biomarker candidates on plasma samples from patients at various stages of the GC cascade, using three complementary approaches: i) the quantification by enzyme-linked immunosorbent assay (ELISA) of plasmatic level of relevant factors, selected according to their role in the host response to H. pylori infection, inflammation and oncogenesis, ii) a proteome profiler ELISA-based analysis of oncology pathways-related factors (84 proteins) and iii) a large-scale screening of plasma proteins by mass spectrometry-based proteomics (MS). Their data led to propose a list of biomarker candidates and signatures allowing to distinguish between healthy subjects and patients, especially those at pre-neoplastic and cancer stage. The characterization of these biomarker signatures paves the way to the development of a diagnostic test that would permit by a simple blood sampling not only the early and easy detection of patients at risk of GC but also their personalized clinical follow-up.

Interestingly, inventors' findings allowed determining sets of biomarkers that may, when used together (within so-called “signatures”), display a strong and reliable capacity to predict gastric pre-cancer condition(s) at various stages or a gastric cancer condition, or to predict the same in a simultaneous fashion (i.e., display a capacity to discriminate between patient health status and/or disease stage(s)). Gastric pre-cancer condition(s) or gastric cancer condition may be identified by stages, especially AG/P and/or GC stage(s), as defined herein. Such a tool may be of particular relevance in the monitoring of patients, especially asymptomatic patients.

Of note, present description may refer to “a gastric cancer condition” as encompassing “a gastric cancer pre-condition” since in some instances the presence of a gastric cancer pre-condition, as defined throughout present description, may precede the advent of gastric cancer condition (GC stage) because gastric cancer pre-conditions can be part of a carcinogenesis process, as detailed herein.

The invention therefore relies on the experiments described herein, and proposes novel means and tools aimed at addressing any one or all of the above-mentioned problems, i.e., provision of easy, reliable and efficient biomarkers enabling determining whether a human patient has lesions rendering said patient at risk of a gastric cancer condition (which is the ultimate stage of the gastric carcinogenesis process discussed herein), i.e., differently said, lesions rendering said patient at risk of developing or at risk of having a gastric cancer condition, and ultimately enabling the early detection of gastric cancer condition, including, according to particular embodiments, diagnosis of the presence of a gastric pre-cancer condition or gastric cancer condition in a patient, with a pertinent accuracy. The invention is in particular aimed at allowing a practician to determine the relevancy to perform further medical or clinical investigations on the patient, in relation to the condition(s) sought. It is an outstanding advantage that instant invention can be carried out on blood or on plasma, of samples drawn from patients.

### DETAILED DESCRIPTION OF THE INVENTION

The invention relates to an in vitro method of determining whether a human patient has lesions rendering said patient at risk of a gastric cancer condition and/or needs further medical test in relation thereto, comprising screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, said method comprising the steps of:
a. determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1 (SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level with the proviso that the selected biomarkers do not consist of the association of IL-8 and mtDNA level, optionally determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level,
b. comparing the levels determined in step a. to a control, and
c. if levels of at least two biomarkers as determined and compared in steps a. and b. deviate from the levels of their controls, conclusion is made that the human patient has lesions rendering said patient at risk of a gastric cancer condition, and/or further medical test, especially clinical investigation, is indicated.

For instance, in a particular embodiment, when two biomarkers are assayed, conclusion is made that the human patient has lesions rendering said patient at risk of a gastric cancer condition if the levels of the said two biomarkers deviate from the levels of their controls, respectively.

According to another particular embodiment, when three biomarkers are assayed, conclusion is made that the human patient has lesions rendering said patient at risk of a gastric cancer condition if the levels of at least two biomarkers amongst the said three biomarkers, deviate from the levels of their controls, respectively.

According to a particular embodiment, the invention relates to an in vitro method of determining whether a human patient has lesions rendering said patient at risk of a gastric cancer condition and/or needs further medical test in relation thereto, comprising screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, said method comprising the steps of:
a. determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level,
b. comparing the levels determined in step a. to a control, and
c. if levels of at least two biomarkers as determined and compared in steps a. and b. deviate from the levels of their controls, conclusion is made that the human patient has lesions rendering said patient at risk of a gastric cancer condition, and/or further medical test, especially clinical investigation, is indicated.

For instance, in a particular embodiment, when two biomarkers are assayed, conclusion is made that the human patient has lesions rendering said patient at risk of a gastric cancer condition if the levels of the said two biomarkers deviate from the levels of their controls, respectively.

According to another particular embodiment, when three biomarkers are assayed, conclusion is made that the human patient has lesions rendering said patient at risk of a gastric cancer condition if the levels of at least two biomarkers amongst the said three biomarkers, deviate from the levels of their controls, respectively.

The number of biomarkers assayed in a method as disclosed herein can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39.

According to particular embodiments, the number of biomarkers assayed in a method as disclosed herein is between 2 and 6, in particular is 2 (with the proviso that the selected biomarkers do not consist of the association of IL-8 and mtDNA level), 3, 4, 5 or 6.

According to a particular embodiment, the list of biomarkers referred to in present description is: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level, with
the proviso that the selected biomarkers do not consist of the association of IL-8 and mtDNA level.

In the expression “with the proviso that the selected biomarkers do not consist of the association of IL-8 and mtDNA level”, it is intended, through the use of the expression “do not consist of” to state that when only two biomarkers are selected for level determination (step a.) in the context of the method of the invention, these markers cannot be IL-8 and mtDNA level taken together (i.e., without the presence of at least one further biomarker as described herein). While such markers IL-8 and mtDNA level may, in the context of present description, be used together in further combination with one or more further biomarker(s) as described herein, the method described herein does not encompass the use of only these two biomarkers that are IL-8 and mtDNA level together. Alternatively, the expression can be written “with the proviso that the two selected biomarkers do not consist of the association of IL-8 and mtDNA level”.

Therefore, according to another alternative writing applicable throughout the present application, the proviso can be written “with the proviso that the selected biomarkers do not consist of the strict association of IL-8 and mtDNA level”, wherein by «strict association of IL-8 and mtDNA level» it is intended that when used in a method or present in a kit or set of markers (or biomarkers) as defined herein, these specifically recited biomarkers are necessarily associated with at least one additional marker (or biomarker) selected in the list of: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, EGFR, STAT3 and MSLN.

By SAA1(SAA2), it is meant herein the SAA1 biomarker or the SAA2 biomarker or both. These proteins pertain to the same family and have close sequence homology. Therefore, they may be substituted to one another or be found concomitantly. Therefore, the expression SAA1(SAA2) in the present description can be substituted by “SAA1” or by “SAA2” or by “SAA1 or SAA2” or by “SAA1 and SAA2” in any occurrence, unless irrelevant from the context.

Of note, the terms “marker” and “biomarker” are used interchangeably herein (synonyms).

According to a particular embodiment, if the biomarkers used allows for such a conclusion, as further described herein in any disclosed aspect, notably regarding disclosed thresholds for decision making, the in vitro method of the invention conversely enables determining whether a human patient is in healthy status, i.e., in present context, whether a human patient does not have lesions rendering said patient at risk of a gastric cancer condition and/or needing further medical test in relation thereto. Such a method comprises the same steps as described above, where in step c. if the levels of at least two biomarkers as determined and compared in steps a. and b. deviate from the levels of their controls, conclusion is made that the human patient does not have lesions rendering said patient at risk of a gastric cancer condition, and/or does not need further medical test, especially clinical investigation.

According to a particular embodiment, the invention relates to an in vitro method of determining whether a human patient has lesions rendering said patient at risk of a gastric cancer condition and/or needs further medical test in relation thereto, comprising screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, said method comprising the steps of:
a. determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, EGFR, STAT3 and MSLN,
b. comparing the levels determined in step a. to a control, and
c. if levels of at least two biomarkers as determined and compared in steps a. and b. deviate from the levels of their controls, conclusion is made that the human patient has lesions rendering said patient at risk of a gastric cancer condition, and/or needs further medical test in relation thereto, especially clinical investigation.

In a more particular embodiment, the method comprises in step a. the determination of the level of at least two biomarkers amongst PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, EGFR, STAT3 and MSLN, where one of the selected biomarkers is replaced by either IL-8 or mtDNA level.

By “lesions rendering said patient at risk of a gastric cancer condition”, it is meant any lesion(s) as defined above and herein, whose gravity is gradually increasing depending upon the stage in the gastric carcinogenesis process which is the subject of instant application. For instance, lesions may be present as early as in a gastric pre-cancer stage (which is assimilated to “gastric pre-cancer condition” herein). For example, lesions in the non-atrophic gastritis (abbreviated NAG herein) stage can be those of a chronic inflammation of the gastric mucosa associated with high production of oxidative species. Lesions in the atrophic gastritis (AG) stage can be the loss of gastric glands. Lesions in the pre-neoplasia (P) stage can encompass a change of gastric epithelial cells which acquire an intestinal cells phenotype, or a specific irregular architecture of the glands. Exemplary lesions at the gastric cancer stage are provided later on in the course of present description.

By “at risk of a gastric cancer condition” in “lesions rendering said patient at risk of a gastric cancer condition”, it is alternatively said “lesions rendering said patient at risk of developing or at risk of having a gastric cancer condition” and it is meant that the assayed patient for which the condition set in step c. is met is: at risk of developing gastric cancer or is at risk of being in an ongoing process of gastric carcinogenesis, the latter of which encompasses several stages of increasing severity (which can be diagnosed as gastric cancer pre-condition(s)), even if reversal of the condition can be seen at any stage.

According to a particular embodiment, by a “deviation of the levels of at least two biomarkers from the levels of their respective controls”, it is meant a deviation that is statistically significant with respect to a control (the control can be a standard for an healthy status or another well-defined status, as long as the change is deemed significant, per common practice in the field for determining significance, especially statistical significance, of a change), as further detailed herein. Indeed, as exposed in instant application, the inventors could determine that variations of biomarkers of present invention are associated with the presence of lesions rendering the patient subjected to the test at risk of a gastric cancer condition and/or needing further medical test in relation thereto. In a particular aspect, the invention therefore seeks a first appreciation of whether subsequent investigation should pertinently be sought for the assayed patient, in connection with a risk of presence of an ongoing gastric carcinogenesis process.

According to a particular embodiment, further medical test(s) which can be indicated correspond to further clinical investigation, defined as clinical research for which an investigator directly interacts with patients in either an outpatient or inpatient setting. This definition may include performance of further in vitro testing, such as performance of various blood tests, e.g., Complete Blood Count (CBC) to check for anemia, but does also go beyond studies for which material of human origin is obtained through a third party and for which an investigator has had no direct interaction with the patient. Non-limitative examples of “further clinical investigations” therefore encompass other investigations methods aimed at confirming or excluding the presence of a gastric carcinogenesis process such as optical gastroscopic examination, computed tomography (or CT) scanning of the abdomen, biopsies for histological examination, the latter of which allows for a precise diagnosis of the type of lesion(s), cancer(s) if any, and/or stage(s) reached in a carcinogenesis process.

If gastric lesions are suspected, further clinical investigation may encompass increasing the number and/or frequency of scheduled optical gastroscopic examinations, which would otherwise have been conducted less frequently.

The determination of a “deviation of the levels of biomarkers with respect to the levels of the control” can be made by any means suitable to this end. In particular, in order to determine “deviation” it can be assessed whether the level determined in step a. for one particular biomarker is increased or decreased with respect to a reference value (i.e., a cut-off value) measured in a control individual or provided as a control value such as one obtained from pooled values of control individuals. A particular example of such a determination is shown in Table 2 herein. According to another embodiment, depending on the direction of the change and/or the absolute value of the change, e.g., expressed as a ratio or in folds, and if needed by comparison with known directions of change for the considered analyzed condition (gastric cancer condition or gastric pre-cancer condition), for example as shown herein in Table 7, one can conclude regarding whether the patient has lesions rendering said patient at risk of a gastric cancer condition and/or needs further medical test in relation thereto, on the basis of the decision rule set in step c. above. Indeed, as shown herein, experiments such as non-targeted mass spectrometry experiments, rendering measured values that are relative values, can still provide a statistically significant information, which is the variation of the level of a biomarker (that may be expressed a ratio) in a relative fashion between stages of disease. Conversely, measurements can also be carried out using quantitative (targeted) mass spectrometry measurements, which provide absolute values that may be compared with one another instead of comparing variation between relative values, as rendered necessary using non targeted mass spectrometry experiments, an example of which is shown in the experimental section herein. Instant invention can be carried out whenever the deviation for the parameters to be assayed, as defined herein, are determined with respect to an absolute reference value, or determined using a comparison between variations of pooled values between distinct disease stages or health status. Such variations may be expressed as ratios. Examples are provided in the experimental section herein.

According to a particular embodiment, step a. defined above consists of determining the level of at least three biomarkers, encompassing the two biomarkers that are IL-8 protein and mtDNA level, in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, and MSLN, optionally determining the level of at least three biomarkers, encompassing the two biomarkers that are IL-8 protein and mtDNA level in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, EGFR, STAT3 and MSLN.

According to particular embodiments, step a. can encompass determining:
the level of PGK1 in further combination with one or more biomarker(s) selected amongst: CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of CFP in further combination with one or more biomarker(s) selected amongst: PGK1, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of IGFALS in further combination with one or more biomarker(s) selected amongst: PGK1, CFP, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KRT19 in further combination with one or more biomarker(s) selected amongst: PGK1, CFP, IGFALS, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of SPRR1A in further combination with one or more biomarker(s) selected amongst: PGK1, CFP, IGFALS, KRT19, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of CPA4 in further combination with one or more biomarker(s) selected amongst: PGK1, CFP, IGFALS, KRT19, SPRR1A, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of CA2 in further combination with one or more biomarker(s) selected amongst: PGK1, CFP, IGFALS, KRT19, SPRR1A, CPA4, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of SERPINA5 in further combination with one or more biomarker(s) selected amongst: PGK1, CFP, IGFALS, KRT19, SPRR1A, CPA4, CA2, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of MAN2A1 in further combination with one or more biomarker(s) selected amongst: PGK1, CFP, IGFALS, KRT19, SPRR1A, CPA4, CA2, SERPINA5, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KIF20B in further combination with one or more biomarker(s) selected amongst: PGK1, CFP, IGFALS, KRT19, SPRR1A, CPA4, CA2, SERPINA5, MAN2A1, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of SPEN in further combination with one or more biomarker(s) selected amongst: PGK1, CFP, IGFALS, KRT19, SPRR1A, CPA4, CA2, SERPINA5, MAN2A1, KIF20B, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of JUP in further combination with one or more biomarker(s) selected amongst: PGK1, CFP, IGFALS, KRT19, SPRR1A, CPA4, CA2, SERPINA5, MAN2A1, KIF20B, SPEN, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KRT6C in further combination with one or more biomarker(s) selected amongst: PGK1, CFP, IGFALS, KRT19, SPRR1A, CPA4, CA2, SERPINA5, MAN2A1, KIF20B, SPEN, JUP, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of CDSN in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KPRP in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of F13A1 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of SAA1(SAA2) in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level. Of note SAA1(SAA2) in the context of present paragraph means either “SAA1” or “SAA2”—but in that case the other biomarker that is SAA1 or SAA2 respectively, that is missing from the list, can be added, or SAA1(SAA2) in the context of present paragraph can mean “SAA1 and SAA2”;
the level of LBP in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of DSP in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KRT2 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of KRT14 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, ARG1, S100A12, ATAD3B, MAN1A1, HAL, DCD, C7, HP, LEP, IL-8, IL-17, TNF-alpha, USF1, USF2, SELE, MSLN, and mtDNA level;
the level of ARG1 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, S100A12, ATAD3B, MAN1A1, HAL, DCD, C7, HP, LEP, IL-8, IL-17, TNF-alpha, USF1, USF2, SELE, MSLN, and mtDNA level;
the level of S100A12 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, ATAD3B, MAN1A1, HAL, DCD, C7, HP, LEP, IL-8, IL-17, TNF-alpha, USF1, USF2, SELE, MSLN, and mtDNA level;
the level of ATAD3B in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, MAN1A1, HAL, DCD, C7, HP, LEP, IL-8, IL-17, TNF-alpha, USF1, USF2, SELE, MSLN, and mtDNA level;
the level of MAN1A1 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, HAL, DCD, C7, HP, LEP, IL-8, IL-17, TNF-alpha, USF1, USF2, SELE, MSLN, and mtDNA level;
the level of HAL in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, DCD, C7, HP, LEP, IL-8, IL-17, TNF-alpha, USF1, USF2, SELE, MSLN, and mtDNA level;
the level of DCD in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, C7, HP, LEP, IL-8, IL-17, TNF-alpha, USF1, USF2, SELE, MSLN, and mtDNA level;
the level of C7 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, HP, LEP, IL-8, IL-17, TNF-alpha, USF1, USF2, SELE, MSLN, and mtDNA level;
the level of HP in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, LEP, IL-8, IL-17, TNF-alpha, USF1, USF2, SELE, MSLN, and mtDNA level;
the level of LEP in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, IL-8, IL-17, TNF-alpha, USF1, USF2, SELE, MSLN, and mtDNA level;
the level of IL-8 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE and MSLN;
the level of IL-17 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8TNF-alpha, USF1, USF2, SELE, MSLN, and mtDNA level;
the level of TNF-alpha in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, USF1, USF2, SELE, MSLN, and mtDNA level;
the level of USF1 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF2, SELE, MSLN, and mtDNA level;
the level of USF2 in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, SELE, MSLN, and mtDNA level;
the level of SELE in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, MSLN, and mtDNA level;
the level of MSLN in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, and mtDNA level;
the level of mtDNA in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE and MSLN;
the level of IL-8 and mtDNA level in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, MSLN and SELE.

According to particular embodiments, biomarkers EGFR and STAT3 are added to the one or more biomarker(s) of the above lists, to the proviso that when the level of IL-8 and mtDNA level are determined altogether, the level of at least one further biomarker is also determined.

According to a particular embodiment, “one or more” means a total of 3, 4, 5 or 6 biomarker(s), i.e., “more” means 2, 3, 4, 5 or 6.

In a particular aspect, instant invention does not encompass the embodiment where the selected biomarkers consist of the strict association of IL-8 and mtDNA level.

According to particular embodiments, the assayed biomarkers are as shown in the combinations depicted in any of Tables 4, 5, 6, 7, 8, 9, 10, 11 or 12 and/or FIG. 9, 11, 13 or 15.

According to particular embodiments, the assayed biomarkers encompass at least one, amongst the selected biomarkers, of S100A12, KIF20B, ARG1, DSP1 or HAL. S100A12 has been shown to be relevant for gastric cancer risk assessment, and KIF20B, ARG1, DSP1 and HAL have been shown to be relevant for AG/P stage assessment. When selected, one or several of these markers can be associated in any combination of biomarkers as described herein.

According to a particular embodiment, step a. of the method of the invention consists of determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level, optionally with at least one further biomarker selected amongst: EGFR and STAT3. In particular embodiment, step a. of the method of the invention consists of determining the level of at least three biomarkers, where at least two biomarkers are selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level, optionally where the third biomarker is selected amongst: EGFR and STAT3.

According to a particular embodiment, step a. of the method of the invention consists of determining the level of at least three biomarkers, including two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level, and at least one further biomarker selected amongst: EGFR and STAT3.

According to a particular embodiment, step a. of the method of the invention consists of determining the level of at least three biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level.

According to a particular embodiment, step a. of the method of the invention consists of determining the level of at least two, preferably between two and six, biomarkers selected amongst: IGFALS, KRT19, CPA4, CA2, MAN2A1, KIF20B, JUP, F13A1, LBP, KRT14, ARG1, S100A12, ATAD3B, DCD, HP, LEP, IL-8, IL-17, TNF-alpha, USF1, USF2, SELE, MSLN, EGFR and STAT3.

According to a particular embodiment, step a. of the method of the invention consists of determining the level of at least two, preferably between two and six, biomarkers selected amongst: IGFALS, KRT19, CA2, MAN2A1, KIF20B, JUP, LBP, ARG1, S100A12, ATAD3B, DCD, HP, LEP, IL-8, IL-17, TNF-alpha, USF1, USF2, SELE, MSLN, EGFR and STAT3, as shown for example in FIG. 13 and Table 12 (biomarkers determined through ELISA experiments).

According to a particular embodiment, step a. of the method of the invention consists of determining the level of at least two, preferably between two and six, biomarkers selected amongst: IGFALS, KRT19, CA2, MAN2A1, JUP, ARG1, S100A12, HP, LEP, IL-17, TNF-alpha, USF2, SELE, MSLN and EGFR.

According to a particular embodiment, deviation of the levels of at least two biomarkers from the levels of respectively corresponding standard is determined using a comparison between variations of pooled values between distinct disease stages/health status: for example, Table 7 shows variations of protein levels between distinct disease stages/health status, expressed as ratios (which can be converted to fold changes, see instant description). It can be seen that for DCD protein, it has been observed an average log2 ratio change of 0.76 between healthy and pre-neoplasia pools of patients, which has been found to be associated with a p-value of 2.54E-02. This means that this ratio change (direction and, roughly, magnitude of change) is deemed significant to conclude that we are in presence of a deviation of the level of DCD protein with respect to healthy patients. Similarly, it can be seen that for LEP protein, it has been observed an average log2 ratio change of −0.64 between healthy and pre-neoplasia pools of patients, which has been found to be associated with a p-value of 5.91 E-02. This means that this ratio change (direction and, roughly, magnitude of change) is deemed significant to conclude that we are in presence of a deviation of the level of LEP protein with respect to healthy patients. In the situation where both DCD and LEP protein levels are assayed, and both parameters show a deviation within the directions and, roughly, magnitude of change shown above with respect to the average value of an healthy pool of patients taken as a reference value, it can be said that the levels of the said two biomarkers deviate from the levels of their controls, respectively, and conclusion can be made that the patient from which the sample was taken is at risk of a gastric cancer condition in the sense that the observed variations indicate a risk of gastric pre-neoplasia. This example is provided to illustrate the teachings that can be drawn from Table 7: such a reasoning can be made for all depicted biomarkers and assayed conditions. Table 8 also provides values for plasmatic levels of biomarkers identified in signatures to predict preneoplasia and GC, as an exemplary reference for the skilled person in the art, which can readily be compared to standard values that can be gathered from samples of individuals determined to be healthy for the tested condition.

According to a particular embodiment, applicable in any part of present description, and in line with conventional practice in the field of medical statistics, a P-value is considered to define a statistically significant test when the value is inferior to 0.05 (P-value<0.05), in some instances, which can be appreciated by the skilled person, if inferior or equal to 0.05.

According to particular embodiments, each assayed biomarker can be included in a decision rule that can be associated, for an assayed condition or one condition amongst several conditions, with an AUC value that indicates a significant predictive power if it is superior to 0.5, and up to perfect predictions if it is equal to 1. According to particular embodiments, the assayed biomarkers provide a test that can be determined to be associated, for an assayed condition, with an AUC value of at least 0.5, preferably at least 0.6, or at least 0.65, or at least 0.7, or at least 0.75, or at least 0.8, or at least 0.85, or at least 0.9, or at least 0.95, in particular an AUC value of 1. Means of calculating AUC values are known to the skilled person in the art and thorough guidance is provided in instant description. For combination of several markers, AUC values may still be calculated using, first, the estimation of a multinomial logistic regression model (see experimental section for an exemplary, non-limiting, protocol), followed by determination of a decision rule based on the estimated multinomial logistic regression model (see in instant description). Additionally, a statistic enabling to determine how well the model fits all disease stages can be used by the residual deviance criterion. Reference is made to the experimental section for exemplary guidance.

According to a particular embodiment, especially but not necessarily where the in vitro method described herein comes with specificity and sensibility parameters which can be attributed to the test in light of one or several cut-off values, as described herein, the in vitro method described herein can be said to be for prognosing or diagnosing a gastric cancer condition or gastric pre-cancer condition. Examples of possible decision outcomes, associated with specificity and sensibility parameters, are provided herein (e.g., data summarized in Table 2 for single parameters or double measurements, or ELISA results shown in Tables 4 or 5, for double or triple measurements. Specificity and sensibility may vary depending upon the cut-off value used for the decision and can be defined in an optimized way using ROC curves).

“A human patient susceptible of suffering from a gastric cancer condition or gastric pre-cancer condition or susceptible of suffering from condition(s) susceptible to evolve in a gastric cancer condition” can encompass a patient presenting risk factors for developing gastric cancer, for example because of physical clues, family history or complaint(s) indicating to the practitioner that an etiology of gastric cancer may be present or may become present. It also includes patients with gastroesophageal reflux, with chronic gastric pain as well as H. pylori seropositive subjects or H. pylori seronegative subjects, which have been beforehand eradicated for H. pylori infection. This definition also encompasses individuals having condition(s) susceptible to evolve in a gastric cancer condition or a declared gastric cancer condition, under treatment or not, which should be monitored. A particular group of patients eligible for the performance of the method of the invention is a group of patients with chronic inflammation associated with gastritis, or patient(s) with gastroesophageal reflux, with chronic gastric pain as well as H. pylori seropositive subjects. As mentioned above, a “patient susceptible of suffering from a gastric cancer condition or susceptible of suffering of condition(s) susceptible to evolve in a gastric cancer condition” may conversely also be H. pylori negative at the time of the sampling and/or testing because of a successful eradication of the infection. The above definition also includes patients previously diagnosed for chronic atrophic gastritis or other gastric lesion that need a clinical follow-up. However, this definition also encompasses patients that are totally asymptomatic with regards to any clinical clues generally associated with gastric cancer, since instant invention is aimed at an early detection of the same, such as stage of molecular disease, based on the determination of blood or plasmatic biomarkers levels, easily and without warning clinical signs.

By “gastric cancer condition” it is meant gastric cancer as conventionally diagnosed according to the medical practice. It includes both cardia (upper stomach) and non-cardia (mid and distal stomach) cancer. “Gastric cancer” may encompass: diffuse gastric adenocarcinoma, intestinal gastric adenocarcinoma and MALT lymphoma. MALT lymphoma (or MALToma) is a form of lymphoma involving gastric mucosa-associated lymphoid tissue (MALT). MALT lymphoma is frequently associated (but not in all cases) with a chronic inflammation resulting from the presence of H. pylori, or linked with the presence of H. pylori. Gastric adenocarcinoma is a malignant epithelial tumor, originating from glandular epithelium of the gastric mucosa. It represents a major proportion of GC, i.e. more than 90% of diagnosed GC are adenocarcinomas. The two types of gastric adenocarcinoma: intestinal type or diffuse type are based on a histological distinction. Different stages be associated to GC using known classifications systems, e.g. the TNM Classification of Malignant Tumours staging system that describes the extent of a patient's cancer. Using this type of classification, one can for instance distinguish between Stages 0, I, II, Ill or IV. In Stage 0, the gastric cancer is limited to the inner lining of the gastric mucosa and may be treatable by surgery when found very early, without need for chemotherapy or radiation treatments. In Stages I and II, the disease has penetrated the deeper layers of the gastric mucosa, and may be treated by surgery, sometimes associated with chemotherapy and/or radiation treatments. In Stage III, the disease may have penetrated other nearby tissues distant lymph nodes. In Stage IV, the disease has spread to nearby tissues and more distant lymph nodes, or has metastasized to other organs. Gastric Cancer is generally abbreviated “GC” in present description, unless indicated otherwise or unless the context dictates otherwise.

GC of intestinal-type, which is often, but not always, induced by H. pylori infection, develops through a sequence of precursor lesions. By “gastric pre-cancer condition” it is thus meant events ranging from non-atrophic gastritis (abbreviated NAG herein) corresponding to a chronic inflammation of the gastric mucosa associated with high production of oxidative species, or atrophic gastritis (AG) to pre-neoplasia (P) as described in Correa and Piazulo, J. Dig. Dis, 2012, 13: 2-9. AG is the first recognizable step of the precancerous cascade corresponding to a loss of gastric glands. Pre-neoplasia encompasses intestinal metaplasia (IM) and dysplasia, before entering gastric cancer stage. IM and dysplasia are recognized as pre-neoplastic lesions. IM correspond to a change of gastric epithelial cells which acquire an intestinal cells phenotype. It is a condition that predisposes to malignancy. Dysplasia are also referred as non-invasive neoplasia with a specific irregular architecture of the glands.

Since, as stated above H. pylori infection is a major risk factor for GC, “condition(s) susceptible to evolve in a gastric cancer condition” encompass for example H. pylori infection in a patient.

According to a particular aspect, the invention more precisely seeks the assessment of a risk according to present description, especially a risk that a human patient has to develop a gastric cancer condition or a risk that a human patient has to have a gastric cancer condition, in particular seeks the prognosis or diagnostic of a gastric pre-cancer condition affecting the tested individual, just before gastric cancer, i.e., at the atrophic gastritis (AG) to pre-neoplasia (P) stages or conditions, also referred to as AG/P herein, by reference to the cohort of patients studied to this effect.

The invention is therefore also for assessing the risk that a human patient has an atrophic gastritis/pre-neoplasia (AG/P), in particular concerns a method which is for prognosing or diagnosing an atrophic gastritis/pre-neoplasia (AG/P) condition in the tested patient.

In a specific embodiment, where outcome allows, the invention is for prognosing or diagnosing an atrophic gastritis/pre-neoplasia (AG/P) condition in the tested patient.

In a particular embodiment, where outcome allows, the invention is for assessing the risk that a human patient has a non-atrophic gastritis (NAG), or an atrophic gastritis/pre-neoplasia (AG/P), or a gastric cancer (GC), in particular the invention concerns a method which is for discriminating between the presence of a non-atrophic gastritis (NAG), an atrophic gastritis/pre-neoplasia (AG/P), a gastric cancer (GC) or an healthy status in the tested patient, in particular a method which is for prognosing or diagnosing one or the other of these conditions, especially simultaneously.

The methods described herein are based on the detection or monitoring of the biological parameters of a patient, and/or allow for the providing of information about the health status of such a patient. When sensitivity/sensibility values can be associated to the test made, the methods of the invention can be defined as enabling the diagnosis, of the sought condition.

Accordingly, the investigation methods described herein enable at least determining a risk according to a non-statistic definition, or a possibility of onset or a risk of presence of gastric pre-cancer condition in a patient, a sample of which is assayed according to the methods described herein. According to a different embodiment, said determination amounts to a prognosis or diagnosis of a gastric pre-cancer condition in a patient, when comparison of the gathered values with relevant control values enables to conclude about the presence or absence of the gastric pre-cancer condition as a direct result, if relevant without recourse to additional tests or clinical investigations. According to the common practice in the field, the sensitivity/sensibility values associated with such a decision, are in line with the choice of the threshold value retained, which can be optimized according to common knowledge in the field, notably by the use of ROC curves, as it will be discussed hereafter.

According to a particular embodiment, the method of the invention is carried out on a blood sample removed from a human patient suffering from a gastric cancer condition or gastric pre-cancer condition or suffering of condition(s) susceptible to evolve in a gastric cancer condition.

In a particular embodiment, the invention is based on the measure of plasmatic biomarkers. Thus, the biological sample can be a blood sample, a plasma sample or a serum sample.

When protein biomarkers are sought, the invention is advantageously carried out on the basis of a biological plasma sample previously obtained from a human subject. This sample can be isolated (collected, removed) from an individual who is susceptible of suffering from a gastric cancer condition or gastric pre-cancer condition or susceptible of suffering from condition(s) susceptible to evolve in a gastric cancer condition as defined above. The individual may or may not have been previously diagnosed for a gastric cancer or for lesions that may lead to a gastric cancer (pre-neoplastic condition or gastric pre-cancer condition) and who, optionally, may have been subjected to a treatment, such as surgery and/or chemotherapy and/or radiations treatment.

A plasma sample is the liquid part of a blood sample which carries the cells and proteins the blood contains. Of note, blood serum is blood plasma without clotting factors. According to a particular embodiment when relevant, the invention can also be carried out on a sample which is a blood serum sample.

While the invention focuses on the measurement of protein levels found in the plasma of an individual, according to a particular embodiment mitochondrial DNA (abbreviated “mtDNA” herein) levels may also be measured. This is conveniently done by testing the mtDNA of leukocyte(s) found in the blood (albeit not in a plasma sample, which does not contain cells). However, since it is found in the circulating blood, the considered mtDNA level can also be said to be a “plasmatic” biomarker. According to a preferred embodiment when mtDNA level is measured, the level of mtDNA is determined by testing circulating blood mtDNA, in particular is determined by testing the mtDNA of leukocyte(s).

According to a particular embodiment, the biological sample removed from the tested individual is a blood sample. According to a more particular embodiment, such a blood sample is beforehand treated to isolate leukocytes from which total DNA is prepared and purified. A measure of mtDNA level can then be performed on the retrieved preparation of total DNA of the leukocytes, if necessary in parallel with the measure of other plasmatic biomarkers as found in the plasma of the same sample, which contained the isolated leukocytes.

The invention is more particularly based, as defined in step a. above, at least on the measurement of the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN protein levels, optionally with the proviso(s) described in any embodiment disclosed in present description.

These markers being proteins, they can be conveniently measured by enzyme-linked immunosorbent assay (ELISA) testing, or, alternatively, by mass spectrometry on the basis of the sample obtained from the individual to be tested. According to another embodiment, they can be measured through Luminex experiments, according to the guidance available to the skilled person in the literature.

Some of those proteins have been determined through a large-scale screening of plasma proteins by mass spectrometry (MS)-based proteomics, to be pertinent targets for the purpose of invention. In particular embodiments, the combinations of proteins shown in Table 6 or FIG. 11 herein, are combinations of at least two plasmatic biomarkers of interest, in particular three plasmatic biomarkers of interest, associated with good predicted AUC values as determined using the protocol shown in the experimental section, for discriminating between a risk of being in the presence of either a NAG, or AG/P or a GC stage or condition in tested individuals. The same is provided with respect to so-called “ELISA” biomarkers including mtDNA levels, in FIG. 9. Some biomarkers have been confirmed through ELISA, and promising biomarkers are recapitulated herein—see Tables 8 to 12.

Levels of proteins in plasma can be measured using commonly known methods in the art such as, as a non-limitative example, through enzyme-linked immunosorbent assay(s) (ELISA) (Engvall E and Perlman P, 1971, Immunochemistry, 8: 871-874). Examples of the same are provided in the experimental section herein (e.g., plasmatic levels of different selected biomarker candidates could be evaluated through commercial ELISA assays from Duo Set RD system or MyBioSource companies. Interleukin-8 (IL-8) (Ref DY208); Interleukin-17 (IL-17) (Ref DY317); Tumor necrosis factor-α (TNF-α) (Ref DY210); Mesothelin (MSLN) (Ref DY3265); E-Selectin (SELE) (Ref DY724); Haptoglobin (HP) (Ref DY8465); Leptin (LEP) (Ref DY398) and Upstream stimulating factor 1 and 2 (USF1 Ref MBS9342772 and USF2 Ref MBS9321077; MyBioSource)). The skilled person can readily determine and retrieve proper ELISA reagents for measuring the desired protein level in a sample.

A Luminex assay is a bead-based simplex or multiplex immunoassay system in a microplate format. In a multiplex form, it can simultaneously detect many targets in a single sample, e.g., up to 500 targets, while the agents enabling capture of the targets, which are coupled to the beads, can be of different types (in a single assay), i.e., proteins including antibodies, ligands, and nucleic acids specific to the desired targets. The beads, which can be used in a Luminex assay can have different spectral addresses (so-called “color-codes”), for example by internally labelling beads with different ratios of two fluorophores. The beads can also be magnetic or non-magnetic. In a Luminex assay, the sample to be analyzed is added to a mixture of color-coded, magnetic or non-magnetic, beads, pre-coated or to be coated with analyte-specific capture agents, such as antibodies. For instance, if the agents are antibodies, biotinylated detection antibodies specific to the analytes of interest are added and form an antibody-antigen sandwich, after the antibodies have bound to the analytes of interest. Phycoerythrin (PE)-conjugated streptavidin can be added to bind the biotinylated detection antibodies. The beads are then read on a dual-laser flow-based detection instrument, i.e., one laser classifies the bead and determines the analyte that is being detected. The second laser determines the magnitude of the PE-derived signal, which is in direct proportion to the amount of analyte bound. Magnetic beads can also be used to holds the magnetic beads in a monolayer, while two spectrally distinct lights, for example emitted by light-emitting diodes (LEDs) illuminate the beads. One light identifies the analyte that is being detected and, the second light determines the magnitude of the PE-derived signal. Luminex allows for high-throughput experiments and is powerful when looking for changes in concentrations of multiple targets, as stated above. Kits for carrying out Luminex experiments can encompass capture beads that are or can be conjugated to the capture agents used, such as capture antibodies. The beads can be color-coded beads, and/or magnetic or non-magnetic beads, and/or carboxylated beads. A kit can include an amine coupling kit for attaching capture agents, such as antibodies, to beads if the beads are carboxylated. A kit can include biotinylated antibodies as detection (secondary) antibodies, an/or phycoerythrin (PE)-conjugated streptavidin to reveal biotinylated antibodies. Such assays can be carried out according to the instructions and guidance provided by the manufacturers, such as BioRad, ThermoFischer Scientific, Luminex or R&D Systems, to cite a few, and conventional knowledge in the field, as detailed in the literature.

The manner of “measuring the level” of a protein biomarker can depend upon the technique used for measuring the same. When ELISA testing is used, the level of a protein biomarker can conveniently be given as a concentration (quantitative value), using the common practice in the art. When mass spectrometry is used, levels of protein biomarker in an assayed plasma sample can conveniently be translated in arbitrary values, whether representative of an absolute value (targeted mass spectrometry experiments) or representative of a relative value enabling to determine a variation with respect to a group used for comparison (non-targeted mass spectrometry experiments). Concretely in order to obtain an “arbitrary value” based on actual physical measurement, it is possible, as shown in the experimental section herein, to make use of a proteomic analysis with an array consisting of capture antibodies present on nitrocellulose membranes that were incubated with plasma samples mixed with a cocktail of biotinylated detection antibodies and revealed by streptavidin-horseradish peroxidase. As a result, obtained values for protein presence in the assayed sample could be reflected as intensity values, the latter of which could be further used for statistical treatment of the information. Comparison with intensity values obtained for distinct groups of individuals, including healthy individuals, is readily achievable and well with the purview of the skilled person in the art. According to another embodiment, “measuring the level” of a protein biomarker can make use of quantitative polymerase chain reaction (q-PCR), e.g., through a TaqMan protein assay, enabling for instance, on the basis of the same technique, measurement of both protein levels and level of mtDNA, from plasma and leukocytes retrieved concomitantly from the patient. TaqMan protein assay conventionally allows for sample protein quantitation using real-time PCR and antibodies.

According to a method of the invention, once a measurement of a level of at least two biomarkers according to step a. has been made, and in order to enable assessing a risk according to present description, in particular prognosing or diagnosing a gastric cancer condition or gastric pre-cancer condition, the obtained levels determined in step a. are, as previously discussed, “compared” to a control. A synonym of “control” can be a so-called “standard” value typically obtained for a same assay but for an individual or a pool of individuals that are known to be healthy for the studied condition, or having a particularly determined condition (see Table 7). It is however noteworthy to state that the skilled person can readily determine such a control value, if needed by considering the literature in the art for the concerned studied condition, and also adjust such a “control” value depending upon the precisely studied cohorts, patients, and optimize it for its purpose, notably using the well-known ROC curve technique. The experimental section provide further guidance in this respect. In a particular embodiment of the methods of the invention, the control may be an internal control, e.g. when the patient's health is monitored by testing biological samples at multiple points overtime.

According to a particular embodiment, a “control” value can also be defined as a “threshold” value enabling decision making, i.e., a value deemed to be “normal”. Such a threshold value is generally determined for subjects determined to be healthy. Examples are provided in Table 2. A normal threshold value determined for healthy subjects can be a value found in the literature, i.e. know to be representative of a healthy situation, or a value found by assaying one biological sample from an healthy subject or alternatively found by assaying several biological samples from several distinct healthy subjects, the resulting normal threshold value being then determined as the mathematical mean of the levels values of all the assayed healthy subjects biological samples, or alternatively found by assaying a pool of biological samples from several distinct healthy subjects. By “healthy subject(s)” it is meant subjects that would have no symptoms of gastric disorders or patients referred to for gastroscopy with gastric biopsies corresponding to a normal phenotype. According to a particular embodiment, a “control” value is a value as found in a healthy individual (or a group thereof, see above), which has been determined to be healthy by standard(s) commonly acknowledged to this end by the skilled person in the field of the invention. Is encompassed within such definition control group(s) of healthy volunteer(s) with a negative H. pylori serology and/or asymptomatic individuals with no suspicion(s) for the disease(s) or condition(s) at stake in present invention. Conversely and according to another embodiment, control group(s) for defining values are made of individual who are considered to be healthy in the medical field, by the highest known standards.

Examples of threshold values can be found in the experimental section herein, for example when IL-8, IL-17 and TNF-alpha factors with a decision making rule in ng/mL coming with particular values of sensitivity and specificity for the decision made. Examples are also provided for USF1 and USF2 factors, with the additional definition of an AUC value determined by ROC curve analysis, the determination of the latter being known by the skilled person in the art. FIGS. 3 and 4, also provide examples of determination of ROC curves. ROC curves represent the drawing of True Positive Rates (TPR) in function of False Positive Rates (FPR) obtained by the processing of the data obtained by the experiments carried out, on the basis of decision rules such as “if the biomarker quantity is superior (or, depending upon the configuration, inferior, or superior or equal, or inferior or equal) to the x cut off value, then the patients is to be classified within one of the category H or NAG or AG/P or GC (choose as appropriate)”. This is allowed by the multiplicity of experiments carried out, and readily enables the skilled person on determine an appropriate “control”, “normal”, “cut-off”, “threshold” value as needed for the purpose of decision making. Furthermore, the AUC parameter, i.e., the “Area Under The Roc Curve” parameter is an effective way to summarize the overall diagnostic accuracy of the decision rule. It takes values from 0 to 1, where a value of 0 indicates a perfectly inaccurate decision rule and a value of 1 reflects a perfectly accurate decision rule. If it is inferior to 0.5 it means that the decision rule does not do better than a random decision and is therefore useless. AUC can be computed using rules known by the skilled person and in the literature (Delacour, H., et al., La Courbe ROC (receiver operating characteristic): principes et principales applications en biologie clinique, Annales de biologie clinique, 2005; 63 (2) : 145-54).

From this, the skilled person can readily determine, when relevant, “optimal cut off values”, i.e., control values in the context of the claimed invention, using a compromise between TPR and FPR, in all circumstances.

Of note, the skilled person can also envision defining a “control” value, or simply analyzing the direction of change of the level of a biomarker measured in an individual, if needed by reference to a pool of values (see Table 7) by considering either the number of “fold” of change (or “ratio”) in a measured or in an experimental value, with respect to a “normal” situation (or another “known” condition for the reference group), or simply considering whether the direction of change is identical or different with respect to a known change. By “fold”, it is meant the expression of a change, in particular a number, describing how much a given quantity changes from a normal to a tested value, the normal value being in particular the “normal threshold” and the tested value of the tested sample. For example, a normal value of 30 and a tested value of 60 correspond to a fold change of 2, or in common terms, a two-fold increase. To the contrary, a normal value of 60 and a tested value of 30 correspond to a fold change of 0.5, or in common terms, a 0.5 fold decrease, also referred to as a “minus” two-folds decrease (expressed in negative terms, with a “minus” sign before the number). Fold changes therefore correspond to a ratio of the tested value to the normal value. In other words, the fold change results from the determination of a ratio of the tested value against the normal value.

Table 7 show variation profiles between assayed samples, according to several comparison schemes. When comparisons with healthy samples are made, then the change depicted in that Table with respect to the analyzed parameter, expressed in log2 (ratio of change), can be used as a rule identical to a reference to a “control value”, the ratio/change bearing the same information as the information provided by a comparison of a peculiar value to a control value. Log2 values can be translated into corresponding fold-changes values using the formula 2^{x}. Defining a combination of several parameters may allow, at a first level, to (for instance) distinguish between healthy patients from diseased one, and at other(s) level(s), refine whether the diseased patient(s) pertain to a particular category of patients along the carcinogenesis cascade. All point to point variations shown in Table 7 are part of instant invention, and can be referred to for the change direction they indicate, and the order of magnitude of the fold/ratio they express. They may provide a reference chart in order to identify whether a patient pertains to a particular group along the carcinogenesis cascade.

According to a particular embodiment, the method of the invention is carried out with assayed biomarkers that are:
PGK1 and CFP protein levels, or
KIF20B and SPEN protein levels, or
JUP and KRT6C protein levels, or
JUP and CDSN protein levels, or
JUP and KPRP protein levels, or
F13A1 and SAA1(SAA2) protein levels, or
KRT19 and LBP protein levels, or
DSP and KPRP protein levels, or
DSP and CDSN protein levels, or
KRT2 and CDSN protein levels.

These biomarkers have been shown, with excellent AUC values, to be relevant in association with one another, for the assessment of a risk according to present description or prognosis and diagnosis of the presence of an AG/P condition in a tested individual, as depicted in Table 6 herein.

According to another embodiment, in line with the considerations of FIG. 12, the method of the invention involves assayed biomarkers that include at least KIF20B and SPEN protein levels, alone or in further combination with at least another biomarker selected amongst: PGK1, CFP, IGFALS, KRT19, SPRR1A, CPA4, CA2, SERPINA5, MAN2A1, JUP, KRT6C, CDSN, KPRP, F13A1, SAA1(SAA2), LBP, DSP, KRT2, KRT14, ARG1, S100A12, ATAD3B, MAN1A1, HAL, DCD and C7 protein levels, in particular wherein the assayed biomarkers include at least KIF20B and SPEN protein levels in further combination with at least another protein biomarker selected amongst: KRT19, ARG1, DSP1 and HAL protein level(s). Indeed, the combination of KIF20B and SPEN has been shown to give a good prediction for AG/P or GC in the appended experimental results.

According to a particular embodiment, the method of the invention is carried out with assayed biomarkers that are:
KIF20B, SPEN and KRT19 protein levels, or
KIF20B, SPEN and ARG1 protein levels, or
KIF20B, SPEN and DSP protein levels, or
KIF20B, SPEN and HAL protein levels, or
KIF20B, SPEN and C7 protein levels, or
KIF20B, SPEN and JUP protein levels, or
KIF20B, SPEN and CPA4 protein levels, or
KIF20B, SPEN and SPRR1A protein levels, or
KIF20B, SPEN and MAN2A1 protein levels, or
KIF20B, SPEN and KRT14 protein levels.

These biomarkers have been shown, with excellent AUC values, to be relevant in an association of at least 3 biomarkers, for the assessment of a risk according to present description, in particular the prognosis and diagnosis of the presence of an AG/P condition in a tested individual, as depicted in Table 6 herein. Furthermore and as a further advantage, they come with an optimal residual deviance for predicting simultaneously all pathologies that are NAG, AG/P and GC conditions, respectively, as shown in FIG. 11 B.

According to another embodiment, in line with the considerations of FIG. 12, the invention relates to a method according to any embodiment as described herein, which further comprises determining the level of at least one another biomarker selected amongst: mtDNA level, MSLN, HP, SELE and TNF-alpha protein level(s).

These markers have indeed been shown to be pertinent in order to prognose or diagnose AG/P condition in patients.

Concerning mtDNA levels, the same can be readily determined by testing circulating blood mtDNA, in particular by testing the mtDNA of leukocyte(s) of a sample previously retrieved from a patient. When mtDNA levels are measured along with another biomarker protein level, both are advantageously measured on the basis of a unique sample retrieved from the tested individual, if necessary differently processed depending upon whether DNA measurements or protein level measurements are carried out.

More precisely, concerning determination of a mtDNA, it can be observed that a “level of mtDNA” can either:
represent a “quantitative” value of mtDNA with respect to a standard or a “normal” mtDNA level and in particular be a value aimed at representing, especially quantifying, the amount of mtDNA in the assayed sample, such as the number of copies of mtDNA, in particular the absolute number of copies of mtDNA, or
or represent a relative amount of mtDNA in the tested biological sample, in particular when the determined amount is normalized with respect to a quantity of nuclear DNA (nDNA) or another suitable reference also present in said biological sample, or with respect to normalizing genes or DNA sequences pertaining to nDNA.

In a particular embodiment, it is therefore possible to determine a level of mtDNA without determining an “absolute” quantity of mtDNA in the sample but rather by evaluating this quantity by reference to another parameter.

To obtain a “level of mtDNA”, use can therefore be made of techniques enabling nucleic acids quantification from a biological sample. Such technique enables the determination of the average concentration (or amount) of nucleic acids, i.e., mtDNA within the context of the present invention, present in a sample. Several methods can be used to establish such concentrations (or amounts), including (1) spectrophotometric analysis of nucleic acids and their further quantification and (2) quantification using the measurement of the fluorescence intensity of dyes that bind to nucleic acids and selectively fluoresce when bound, as well as (3) quantification after specific nucleic acids amplification, such as in the real time PCR technique, which also relies on the detection of a fluorescent dye bound to said nucleic acids to be detected and quantified. Preferably, prior isolation of mtDNA to be quantified may be required, according to the common knowledge in the art of nucleic acids analysis.

In particular, mtDNA levels can be determined using quantitative polymerase chain reaction (q-PCR), using conventionally known protocols. Primers specific for the 12sRNA mitochondrial gene may be used, although one skilled in the art can suitably choose other genes or sequences of the mitochondrial genome for implementing such a technique, following guidance available in the literature of this field with respect to this technique. PCR (polymerase chain reaction) is a common method for amplifying DNA. In order to amplify small amounts of DNA, a DNA template, at least one pair of specific oligonucleotide primers, nucleotides (dATP, dCTP, dGTP, dUTP), a suitable buffer solution and a thermo stable DNA polymerase are required. A substance marked with a fluorophore is generally added to one reagent of this mixture in a thermal cycler that contains sensors for measuring the fluorescence of the fluorophore after it has been excited at the required wavelength allowing the generation rate to be measured for one or more specific products. Real-time PCR (q-PCR) is generally applied to the detection and quantification of DNA in samples to determine the presence and/or abundance of a particular DNA sequence in these samples. A measurement is made after each amplification cycle, which enables the quantification of the amplified product in real time.

Real-time PCR is performed by using a real-time PCR apparatus, and after each cycle, the levels of fluorescence are measured with a detector. Used dyes generally only fluoresce when bound to the DNA amplified through PCR, and the increase of fluorescence is detected, corresponding to increasing presence of the amplified products, at each amplification cycle.

Real-time PCR can be used to quantify nucleic acids by either relative quantification or absolute quantification. Absolute quantification gives the exact number of target DNA molecules by comparison with DNA standards using a calibration curve. By this method, it is possible to determine the number of mtDNA copies in patients suspected for the presence of gastric pre-neoplasia or neoplasia and compare this number to the number of mtDNA copies defined in healthy subjects. (Ref: von Wurmb-Schwark et al, 2002, Forensic Science International, 126: 34-39; Fernandes et al, 2014, Cancer Epidemiol Biomarkers Re, 23: 2430-38). Relative quantification enables determining fold-differences between a target sequence, the quantity of which is to be determined, and a “housekeeping sequence”.

In order to quantify the presence of a specific DNA target sequence, representative of the copy number of the mtDNA, it is indeed convenient to express its relative level in relation to another DNA sequence called a “normalizing sequence” or “housekeeping sequence”, which is selected for its almost constant rate of expression. Housekeeping sequences are usually found in genes involved in the functions related to basic cellular survival, which normally implies constitutive gene expression. This enables the provision of a ratio expressing the presence of the amplified sequence of interest over the presence of the amplified selected normalizer. This method allows obtaining a value evaluating the relative presence of the amplified sequence of interest actually knowing its absolute quantity within the tested sample.

Commonly used normalizing sequences are those found in genes coding for the following proteins. As a non-limitative list, tubulin, glyceraldehyde-3-phosphate dehydrogenase, albumin, cyclophilin, ribosomal RNAs sequences can be used.

Real time PCR allows quantification of the desired product at any point in the amplification process by measuring fluorescence. Measurement is expressed using a Cycle Threshold (CT) value (CT; PCR cycle at which the fluorescence of the sequence of interest is detected; the lowest is the CT value, the more abundant is the target sequence). To quantify the presence of the target sequence when a normalization sequence is used, a normalization procedure such as the ΔΔCT-method can be used, said ΔΔCT-method being used for analyzing a relative gene expression.

According to a more specific embodiment, the “level of mtDNA” can be determined by quantitative polymerase chain reaction (q-PCR) through reference to a selected normalizer gene or nDNA sequence, the level of mtDNA being calculated according to the formula 2ΔCt, wherein ΔCt=CtnDNA−CtmtDNA, as described in reference publications Fan et al, 2009, J Cancer Res Clin Oncol, 135; 983-989 and/or Chatre and Richetti, 2013, J of Cell Sciences, 126: 914-926. With this calculation method, the level of mtDNA can be calculated using the ΔCT of average CT of mtDNA and nDNA (ΔCT=CtnDNA−CtmtDNA,) as 2ΔCt. The primers used for amplification can be chosen by one skilled in the art according to the common knowledge in the field of this technique, as indicated in particular in the above-mentioned reference publications.

An example of protocol for determining mtDNA levels can encompass the steps of:
preparing the biological sample to provide access to the nucleic acid, especially mitochondrial nucleic acid of cells;
contacting the prepared sample with oligonucleotide primers targeting the mtDNA;
performing amplification cycles,
simultaneously running amplification of a normalizer nDNA,
Quantitatively detecting the mtDNA and the normalizer nDNA.
determining the level of mtDNA through reference to a selected normalizer nDNA sequence, the level of mtDNA being calculated according to the formula 2ΔCt, wherein ΔCt=CtnDNA−CtmtDNA.

Further details concerning mtDNA measurements especially from leukocytes are available in the experimental section herein, and comprehensive elements can also be found in WO 2015/049372, which is incorporated by reference herein.

As shown in the experimental section herein, an increased level of mtDNA above 6.3 in the conditions of the experiment, allowed to predict patients with AG/P with a sensitivity of 66.6% and a specificity of 65% (AUC value of 0.7089).

As shown in the experimental section herein, the TNF-alpha protein level was interesting with respect to a “not healthy” decision rule, with an elevated concentration of TNF-alpha being significant for assessing that the assayed sample is from a non-healthy patient, with a very high sensitivity and specificity (AUC value of 0.7954).

As shown in the experimental section herein, the MSLN protein level was interesting with respect to two decision rules, in particular a most elevated concentration of MSLN being significant for identifying patients with AG/P (AUC value of 0.7433).

As shown in the experimental section herein, the HP protein level was interesting with respect identification of patients with GC (AUC value of 0.6622).

As shown in the experimental section herein, the SELE protein level was interesting with respect to a “not healthy” decision rule, with an elevated concentration of SELE being significant for assessing that the assayed sample is from a non-healthy patient, with the best AUC value of 0.7565.

As shown in the experimental section herein, the HP plasmatic protein level was interesting because an increased concentration of HP plasmatic protein of 1.7 folds could be found in GC samples.

According to a particular embodiment, the method of the invention is for assessing a risk according to present description, in particular prognosing or diagnosing a non-atrophic gastritis (NAG) condition in the tested patient.

According to a particular embodiment, the method of the invention is for assessing a risk according to present description, in particular prognosing or diagnosing an atrophic gastritis/pre-neoplasia (AG/P) condition in the tested patient. According to this embodiment, the method of the invention is for assessing the risk that a human patient has an atrophic gastritis/pre-neoplasia (AG/P), in particular the method is for prognosing or diagnosing an atrophic gastritis/pre-neoplasia (AG/P) condition in the tested patient.

According to a more specific embodiment, where the assessment of atrophic gastritis/pre-neoplasia (AG/P) is sought, step a. of the method of the invention consists of determining the level of at least two, preferably between two and six, biomarkers selected amongst: IGFALS, KRT19, CA2, MAN2A1, KIF20B, JUP, LBP, S100A12, ATAD3B, DCD, HP, LEP, IL-8, IL-17, USF1, USF2, SELE, MSLN and EGFR, in particular consists in determining the level of IGFALS, KRT19, HP, LEP, MSLN and EGFR.

The selected biomarkers can be 2, 3, 4, 5 or 6.

According to embodiments where assessment of atrophic gastritis/pre-neoplasia (AG/P) is sought and where between 2 and 6 biomarkers are used, are encompassed, in these specific embodiments, the selection of biomarkers as described in any one of the combinations disclosed in Table 10, which lists the best combinations of 2 to 6 biomarkers to predict AG/P selected on the basis of the best AUC value obtained. For AG/P, as soon as 4 biomarkers are used according to the described corresponding signature, AUC is 0.82 and Sensitivity is of 92%.

According to more specific embodiments where assessment of atrophic gastritis/pre-neoplasia (AG/P) is sought and where 6 biomarkers are used, are encompassed, in these specific embodiments, the selection of biomarkers as described in any one of the combinations disclosed in Table 9, which lists the best combinations of 6 biomarkers to predict AG/P: all of these combinations correspond to an AUC≥0.8, with Sensitivity between 90 to 96% and Specificity between 71% and 79%.

According to particular embodiments, which can be combined to any other embodiment described herein, especially embodiments described above where the assessment of atrophic gastritis/pre-neoplasia (AG/P) is sought, the method of the invention, which is for assessing the risk that a human patient has an atrophic gastritis/pre-neoplasia (AG/P), comes with a sensitivity of at least 80%, 81%, 82%, 83%, 84%, 85%, 86%, 87%, 88%, 89%, 90%, 91%, 92%, 93%, 94%, 95%, 96%, 97%, 98%, 99% or 100%, and/or a specificity of at least 80%, 81%, 82%, 83%, 84%, 85%, 86%, 87%, 88%, 89%, 90%, 91%, 92%, 93%, 94%, 95%, 96%, 97%, 98%, 99% or 100%, in particular a sensitivity and a specificity of at least 80%, 81%, 82%, 83%, 84%, 85%, 86%, 87%, 88%, 89%, 90%, 91%, 92%, 93%, 94%, 95%, 96%, 97%, 98%, 99% or 100% each.

According to a particular embodiment, the method of the invention is for assessing a risk according to present description, in particular prognosing or diagnosing gastric cancer (GC) condition in the tested patient.

According to a more specific embodiment, where the assessment of gastric cancer (GC) condition is sought, step a. of the method of the invention consists of determining the level of at least two, preferably between two and six, biomarkers selected amongst: IGFALS, KRT19, CA2, MAN2A1, KIF20B, JUP, LBP, ARG1, S100A12, ATAD3B, DCD, HP, LEP, IL-8, IL-17, TNF-alpha, USF1, USF2, MSLN, EGFR and STAT3, in particular consists in determining the level of ARG1, LEP, IL-17, TNF-alpha, SELE and MSLN.

The selected biomarkers can be 2, 3, 4, 5 or 6.

According to embodiments where assessment of gastric cancer (GC) condition is sought and where between 2 and 6 biomarkers are used, are encompassed, in these specific embodiments, the selection of biomarkers as described in any one of the combinations disclosed in Table 10, which lists the best combinations of 2 to 6 biomarkers to predict GC (cancer lesions) selected on the basis of the best AUC value obtained. For GC, a number of 6 biomarkers allow to improve the sensitivity while the specificity is already excellent (95%) with only one protein (i.e., IL-17).

According to more specific embodiments where assessment of gastric cancer (GC) condition is sought and where 6 biomarkers are used, are encompassed, in these specific embodiments, the selection of biomarkers as described in any one of the combinations disclosed in Table 11, which lists the best combinations of 6 biomarkers to predict GC: all of these combinations correspond to an AUC≥0.9, with Sensitivity between 87 to 94% and Specificity between 71% and 79%.

According to particular embodiments, which can be combined to any other embodiment described herein, especially embodiments described above where the assessment of gastric cancer (GC) condition is sought, the method of the invention, which is for assessing the risk that a human patient has an atrophic gastritis/pre-neoplasia (AG/P), comes with a sensitivity of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%, and/or a specificity of at least 90%, 91%, 92%, 93%, 94%, 95%, 96%, 97%, 98%, 99% or 100%, in particular a sensitivity and a specificity of at least 90%, 91%, 92%, 93%, 94%, 95%, 96%, 97%, 98%, 99% or 100% each.

Let it be when assessment of atrophic gastritis/pre-neoplasia (AG/P) or gastric cancer (GC) condition is sought, or in general, the sensitivity and specificity come with the selection of biomarkers, and can readily be determined by routine practice in the field, with further guidance provided herein. Means of calculating AUC values are known to the skilled person in the art and recalled above in present text. Similarly, specificity and sensibility, which may vary depending upon the cut-off value used for the decision, and can thus be selected according to said cut-off value used for decision, can be defined in an optimized way using ROC curves, as also recalled above in the present text, and done in the Experimental Section herein. The skilled person can therefore readily adjust the biomarkers to be used in order to reach a suitable level of sensitivity and specificity as desired.

According to other embodiments, when assessment of gastric cancer (GC) condition is sought, biomarkers that are or at least one biomarker selected amongst: CA2, KIF20B, ARG1, DCD, LEP, IL-17, TNF-alpha and MSLN have been determined to be promising (Table 3) and can therefore be included in a signature for this purpose.

According to other embodiments, when assessment of atrophic gastritis/pre-neoplasia (AG/P) is sought, biomarkers that are or at least one biomarker selected amongst: IGFALS, KRT19, CA2, MAN2A1, LBP, LEP, SELE, MSLN and EGFR have been determined to be promising (Table 3) and can therefore be included in a signature for this purpose.

According to a particular embodiment, the method of the invention is for assessing the risk that a human patient has a non-atrophic gastritis (NAG), or an atrophic gastritis/pre-neoplasia (AG/P), or a gastric cancer (GC).

According to a particular embodiment, the method of the invention is for assessing a risk according to present description, in particular prognosing or diagnosing a non-atrophic gastritis (NAG) condition or atrophic gastritis/pre-neoplasia (AG/P) condition in the tested patient.

According to a particular embodiment, the method of the invention is for assessing a risk according to present description, in particular prognosing or diagnosing a non-atrophic gastritis (NAG) condition or a gastric cancer (GC) condition in the tested patient.

According to a particular embodiment, the method of the invention is for assessing a risk according to present description, in particular prognosing or diagnosing an atrophic gastritis/pre-neoplasia (AG/P) condition or a gastric cancer (GC) condition in the tested patient.

According to embodiments, when assessment of atrophic gastritis/pre-neoplasia (AG/P) or a gastric cancer (GC) condition is sought, biomarkers that are or at least one biomarker selected amongst: CA2, LEP and MSLN have been determined to be promising for both conditions (Table 3) and can therefore be included in a signature for this purpose, including in a context of discrimination between these conditions.

According to a particular embodiment, the method of the invention is for assessing a risk according to present description, in particular prognosing or diagnosing a non-atrophic gastritis (NAG) condition, or a atrophic gastritis/pre-neoplasia (AG/P) condition or a gastric cancer (GC) condition in the tested patient, i.e., allows discrimination between these situations. The difference between these distinct clinical situations can be made depending upon the observed changes of values with regards to a “normal” situation, i.e., the direction (increase or increase) and extent of the change (the number of folds).

According to a particular embodiment, and in line with the considerations of FIG. 12, the method of the invention further comprises determining the level of at least one another protein biomarker selected amongst: LEP and S100A12 protein level(s), and/or IL-17 protein level, and/or LEP, S100A12 and IL-17 protein level(s).

LEP and S100A12 protein levels have indeed been shown to be pertinent in order to prognose or diagnose GC condition in patients. Accordingly, such biomarkers added to a signature, according to all possible combinations disclosed herein, can come as a further check of whether or not the individual, the sample of which is assayed, may have GC or not.

Conversely, IL-17 protein level has been shown to be pertinent in order to detect healthy individuals, and therefore exclude a gastric cancer condition or gastric pre-cancer condition (see Table 4). As a consequence, such a biomarker added to a signature, according to all possible combinations disclosed herein, can come as a further check of whether or not the individual, the sample of which is assayed, may be healthy.

Such measurements can assist corroborating or invalidating the results obtained with a signature of biomarkers according to instant invention.

According to an aspect, is also disclosed a method which encompasses as a sequence of steps:
i) performance of a method as disclosed herein, where the assayed biomarkers include at least KIF20B and SPEN protein levels, alone or in further combination with at least another biomarker as described herein, in particular wherein the assayed biomarkers include at least KIF20B and SPEN protein levels in further combination with at least another (or more) protein biomarker selected amongst: KRT19, ARG1, DSP1 and HAL protein level(s) and/or mtDNA, MSLN, HP, SEL and TNF-alpha protein level(s); these markers are relevant with regards to the assessment of a risk of presence of an AG/P stage (see FIG. 12) in the assayed patient, and
ii) performance of a method as disclosed herein, where the assayed biomarker is IL-17, in order to corroborate or double-check whether the assayed patient may in fact be considered as healthy (see FIG. 12), and
iii) performance of a method as disclosed herein, where the assayed biomarkers include at least LEP or S100A12 protein levels; these markers are relevant with regards to the assessment of a risk of presence of gastric cancer (see FIG. 12) in the assayed patient. Such a measure can be used to corroborate or double-check whether the assayed patient may in fact be considered as at risk of having gastric cancer,
   where in particular steps ii) and iii) can be carried out in order, or where only one of those steps are carried out, or where only one of steps i) or ii) or iii) are carried out.

According to an embodiment, the method of the invention is for assessing a risk according to present description, in particular prognosing or diagnosing a non-atrophic gastritis (NAG), or an atrophic gastritis/pre-neoplasia (AG/P) gastric cancer condition, or a gastric cancer (GC) in the tested patient, in particular which is for discriminating between a non-atrophic gastritis (NAG), an atrophic gastritis/pre-neoplasia (AG/P) gastric cancer condition, a gastric cancer (GC) or an healthy status in the tested patient.

According to a particular embodiment, the method of the invention is for monitoring or diagnosing the health status of a patient susceptible of suffering from condition(s) susceptible to evolve in a gastric cancer condition or susceptible of suffering from a gastric pre-cancer condition or susceptible of suffering from a gastric cancer condition, or the health status of a human patient that has lesions rendering said patient at risk of a gastric cancer condition, wherein the method is repeated at least once over time so as to conclude about the health status of the tested patient if the comparison set in step b. discussed herein (comparison with a “control”) and/or the deviation observed in step c. of claim 1 shows an evolution, in particular for monitoring or diagnosing the health status of a patient diagnosed with gastric cancer, and optionally treated for gastric cancer.

According to an embodiment, the method is repeated as needed, i.e., repeated as long as there is need to monitor the evolution of the health status of a patient that may be under treatment for its condition, or not.

The method of the invention is for assessing a risk according to present description, in particular prognosing or diagnosing a gastric cancer condition or gastric pre-cancer condition, i.e., is either for diagnosing when possible (within particular specificity and sensitivity values, which can be readily determined by the skilled person as described herein and according to common knowledge), or for prognosing/predicting a risk, in association with further clues whenever required. Indeed, according to the results of the test, further examination of the individual may be recommended in order to better assess the clinical picture.

Accordingly, the method of the invention enables to determine the status of biological parameters of an individual, and, when possible, statistically determine that the individual, the biological sample of which is tested, may present a risk of suffering a gastric cancer condition or gastric pre-cancer condition. In particular, presence of a gastric pre-cancer condition, especially an AG/P condition, comes with a risk that a gastric carcinogenesis is present in the tested individual. Accordingly, further clinical investigations may be ordered. According to a particular embodiment, prognosis or diagnosis require performing further clinical investigation(s), as described herein.

According to a particular embodiment, the fact of concluding that proceeding with further clinical investigation(s) may be required or ordered, corresponds to a conclusion about the health status of a patient from which the tested biological sample has been removed.

Therefore, “concluding about the health status” and/or “proceeding with further clinical investigation(s)” also encompass enrolment of said patient in a procedure of closer therapeutic monitoring, i.e., said patient is recommended with or directly incorporated in a therapeutic follow-up comprising a regular monitoring of his/her condition or health status over time, and optionally further clinical investigations regarding its health status. Precisely, any determination that the tested individual is susceptible of suffering from gastric lesions, also suggests performance of further clinical investigations. To detect the existence of a risk of gastric carcinogenesis at an early stage, i.e., detection of a risk of presence of gastric lesions at a stage such as the one corresponding to an AG/P condition (or, alternatively, AG/P stage), is a pertinent public health objective, as detailed herein.

Non-limitative examples of “further clinical investigations” encompass other investigations methods aimed at confirming or excluding the presence of a gastric carcinogenesis process such as optical gastroscopic examination, computed tomography (or CT) scanning of the abdomen, biopsies for histological examination, various blood tests, e.g., Complete Blood Count (CBC) to check for anemia.

If gastric lesions are suspected, “concluding about the health status” and/or “proceeding with further clinical investigation(s)” may encompass increasing the number and/or frequency of scheduled optical gastroscopic examinations, which would otherwise have been conducted less frequently.

If gastric lesions are suspected, and possibly following further clinical investigations, it is also possible to conclude to chirurgically remove areas suspected for having entered a gastric carcinogenesis process, when possible. In some instances, resection can be carried out by endoscopy (Gastrointestinal endoscopic mucosal resection (EMR) is a procedure to remove early-stage cancer and precancerous growths from the lining of the digestive tract).

FIG. 16 depicts possible procedures/schemes of use of diagnostic test based on the detection of preneoplasia and GC lesions by SIG-AGP and SIG-GC signatures (described in FIG. 15), respectively—see legend of FIG. 16 herein. It can be seen that a diagnostic test of the invention can be used at different levels of the proposed procedure, and reiterated. Accordingly, the method of the invention can be used in an initial diagnosis protocol and/or a follow-up protocol, as described in this Figure.

In a particular embodiment, the method of the invention also comprises as a distinct, simultaneous or parallel step, a step of detecting an Helicobacter pylori infection, in particular through detection of antigen(s) specific for H. pylori infection, or through an assay involving DNA amplification and subsequent detection of said DNA, or detection of the presence of specific H. pylori IgA and IgG antibodies in a biological sample removed from the tested patient, or through an 13C urea breath test performed on the tested patient.

In that case, the detection of H. pylori may be performed, when relevant, on a fraction (aliquot) of the biological sample removed from the patient, in particular on a plasma sample or the serum fraction of a blood sample whenever relevant by carrying out a step of detection of antigens specific for H. pylori infection. People infected by H. pylori have specific IgA and IgG antibodies that can be easily detectable. In addition, the search for the presence of CagA antigens can also confirm the presence of H. pylori. Another method to detect H. pylori is the 13C urea breath test, a non-invasive test with high sensitivity widely used in human medicine (Graham et al, 1987, Lancet, 1: 1174-1177). This respiratory test allows an indirect measure of the H. pylori-associated urease activity. Presence of H. pylori can also be detected in stools by immunoassay indicating the presence of H. pylori antigens or by amplification of H. pylori DNA in particular by polymerase chain reaction (PCR) using specific primers for H. pylori genes sequences, which are available in the literature to one skilled in the art, and detection of the amplified DNA.

When both a mtDNA level and detection of H. pylori are sought altogether, the biological sample obtained from the tested individual may be a blood sample that can be prepared on the one hand to purify the cellular fraction of the blood sample, in particular the mononuclear cells or leukocytes containing the mtDNA to be assayed and on the other hand to collect the serum enabling the detection of H. pylori infection.

In a particular embodiment, the tested biological sample, especially plasma sample, is obtained from a patient diagnosed with gastric carcinogenesis and under treatment for this condition or not, and/or a patient having an ongoing, treated or not, Helicobacter pylori infection, and/or a patient having antecedents of Helicobacter pylori infection(s), eradicated by prior or ongoing treatment or not, and/or an individual having gastric pain and/or a family history of gastric cancer.

As detailed in present description, and according to particular embodiments applicable to all embodiments described herein, the levels of plasmatic biomarkers are determined by enzyme-linked immunosorbent assay (ELISA) testing, or Mass Spectrometry, or quantitative polymerase chain reaction (q-PCR), or Luminex assay, and, when carried out, the level of mtDNA is determined by quantitative polymerase chain reaction (q-PCR).

Another object of the invention is, when protein level(s) or antigenic detection is contemplated, to provide a kit suitable for carrying out a method as defined herein, or a kit for carrying out a method as defined herein, said kit comprising:
at least two types of antibodies having different antigen specificity wherein each type of antibody is specific fora protein selected amongst:: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins or a combination of several antibodies having different antigen specificity for: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins, and, optionally, at least one antibody specific for H. pylori antigen(s), such as CagA antigens, and, optionally one or several of the following reagents,
a secondary antibody, such as a biotinylated antibody, or reagent to reveal a complex between specific antibody(ies) recited above and its(their) target,
optionally, a buffer solution,
optionally, beads such as color-coded beads, and/or magnetic or non-magnetic beads, and/or carboxylated beads, with optionally an amine coupling kit for attaching antibodies to beads,
optionally, phycoerythrin (PE)-conjugated streptavidin to reveal biotinylated antibodies,
optionally, an assay plate, and
optionally a notice providing instructions for use and expected values for interpretation of results.

Another object of the invention is, when nucleic acid detection is contemplated, to provide a kit suitable for carrying out a method as defined herein, or a kit for carrying out a method as defined herein, said kit comprising:
At least one pair of specific oligonucleotide primers, or nucleic acid molecules, specific for hybridization with mtDNA and/or
At least two pairs of specific oligonucleotide primers, or nucleic acid molecules, specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins, and, optionally, at least one pair of specific oligonucleotide primers or nucleic acid molecules specific for hybridization with H. pylori nucleic acid(s) sequence(s), and, optionally, one or several of the following reagents,
nucleotides (e.g. dATP, dCTP, dGTP, dUTP),
a DNA polymerase, in particular a thermostable DNA polymerase, such as a Taq DNA Polymerase,
at least one dye for staining nucleic acids, in particular a dye detectable in a real-time PCR equipment,
optionally, a buffer solution,
optionally, reagents necessary for the hybridization of the primers to their targets,
optionally, a reference dye and,
a notice providing instructions for use and expected values for interpretation of results.

Another object of the invention is, when a combination of protein level(s), antigen(s) and nucleic acid detection is contemplated, according to all possible combinations thereof, a kit suitable for carrying out a method as defined herein, or a kit for carrying out a method as defined herein, said kit comprising:
at least two types of antibodies having different antigen specificity wherein each type of antibody is specific fora protein selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins or a combination of several antibodies having different antigen specificity for PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins, and, optionally, at least an antibody specific for H. pylori antigen(s), such as CagA antigens, and, optionally one or several of the following reagents,
a secondary antibody, such as a biotinylated antibody, or reagent to reveal a complex between specific antibody(ies) recited above and its(their) target,
optionally, a buffer solution,
optionally, beads such as color-coded beads, and/or magnetic or non-magnetic beads, and/or carboxylated beads, with optionally an amine coupling kit for attaching antibodies to beads,
optionally, phycoerythrin (PE)-conjugated streptavidin to reveal biotinylated antibodies,
optionally, an assay plate, and
optionally a notice providing instructions for use and expected values for interpretation of results, and
At least one pair of specific oligonucleotide primers specific for hybridization with mtDNA and/or
At least two pairs of specific oligonucleotide primers specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and, optionally, at least one pair of specific oligonucleotide primers specific for hybridization with H. pylori nucleic acid(s) sequence(s), and, optionally, one or several of the following reagents, optionally, at least one pair of specific oligonucleotide primers or nucleic acid molecules specific for hybridization with H. pylori nucleic acid(s) sequence(s), and, optionally, one or several of the following reagents,
nucleotides (e.g. dATP, dCTP, dGTP, dUTP),
a DNA polymerase, in particular a thermostable DNA polymerase, such as a Taq DNA Polymerase,
at least one dye for staining nucleic acids, in particular a dye detectable in a real-time PCT equipment,
optionally, at least one buffer solution,
optionally, reagents necessary for the hybridization of the primers to their targets,
optionally, a reference dye.

Another object of the invention is indeed to provide a kit suitable for carrying out a method of the invention as defined herein, comprising a combination of some of the agents, or all the agents, mentioned in the above-described kits, i.e., a kit, which includes all or some reagents for the detection of a protein by enzyme like immunoassay (ELISA), or performance of a so-called TaqMan protein assay (qPCR based), and/or specific antibodies allowing to quantify these proteins also including the necessary positive and negative controls to perform the assays when relevant and, optionally, at least one marker specific for H. pylori antigen(s), as well as, when nucleic acid have to be measured (for mtDNA level or determination of the presence of H. pylori DNA or RNA), tubes and/or means allowing the separation of leukocytes and plasma from blood samples, and reagents necessary to isolate DNA from leucocytes and to perform both mtDNA detection and quantification including couples of primers specific to mtDNA and nDNA genes as relevant, or H. pylori gene(s) or RNA as relevant, also including Taq DNA polymerase, deoxynucleotides mix, buffer and dye needed for qPCR reaction, or, according to another embodiment, all agents and/or reagents for carrying out Luminex assays.

Another object of the invention is a set of markers comprising or consisting of at least two antibodies specific fora protein selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and optionally at least one pair of specific oligonucleotide primers or nucleic acid molecules specific for hybridization with mtDNA, or set of markers comprising or consisting of at least two pairs of specific oligonucleotide primers specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and optionally at least one pair of specific oligonucleotide primers specific for hybridization with mtDNA, suitable to carry out a method as defined in any embodiment herein, or for carrying a method as defined in any embodiment herein.

The invention also relates to the use of kit(s) according to the invention or a set of markers as defined herein, for determining whether a human patient has lesions rendering said patient at risk of a gastric cancer condition and/or needs further medical test in relation thereto, or for assessing the risk that a human patient has to develop or the risk the human patient has to have a gastric cancer condition, in particular for prognosing or diagnosing a gastric pre-cancer condition or a gastric cancer condition, by screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, especially by measuring the level of at least two markers as defined in any embodiment herein in a biological blood or plasma sample removed from a human patient susceptible of suffering of condition(s) susceptible to evolve in a gastric cancer condition or susceptible of suffering from a gastric pre-cancer condition or susceptible of suffering from a gastric cancer condition

In a particular embodiment, the invention relates to the use of a kit or a set of markers as defined herein for prognosing or diagnosing a gastric cancer condition or gastric pre-cancer condition, by screening a biological sample of blood or plasma previously removed from a human patient susceptible of suffering from a gastric cancer condition or a gastric pre-cancer condition or susceptible of suffering of condition(s) susceptible to evolve in a gastric cancer condition, especially by measuring the level of at least two markers as defined in any embodiment herein in a biological blood or plasma sample removed from a human patient susceptible of suffering from a gastric cancer condition or a gastric pre-cancer condition or susceptible of suffering of condition(s) susceptible to evolve in a gastric cancer condition.

According to present disclosure, the use of kit(s) according to the invention or a set of markers as defined herein, is to investigate the parameter(s) detailed herein, and/or monitor said parameter(s) in the tested individual, as intermediate biological parameter(s) before any further investigation.

The invention also relates to the use of agents, ingredients or reagents, as described in any aspect disclosed herein, in particular when the kits suitable for implementing the invention are described, for the manufacture of a kit suitable for or aimed at performing the method of the invention as described herein. Instructions for use or guidance for implementing the method of the invention and/or instructions for use or guidance in order to obtain a suitable kit may advantageously be provided.

According to another aspect, it is to be understood that the method of the invention as described in any embodiment herein can be at least partly implemented by a computer. In particular, steps b. and c. of the method of the invention described in any embodiment herein, can be implemented by a computer to which the data corresponding to the level of at least two biomarkers as described in any embodiment herein, is provided as an output. According to another particular embodiment, a computer can also drive the in vitro gathering of the data to be collected in step a., i.e., the collection of the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level, according to any embodiment of levels of biomarkers described herein, through appropriate interfacing means between a computer and level measuring devices.

In line with the possibility that a computer can implement steps b. and c. of the method of the invention described in any embodiment herein, the invention also relates to a method carried out by a computer for investigating whether a human patient has lesions rendering said patient at risk of a gastric cancer condition and/or needs further medical test in relation thereto, the method comprising the steps of:
a. receiving the levels of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level, or receiving any levels of at least two biomarkers whose determination is defined in any embodiment described herein, in particular where the levels of the at least two biomarkers are measured through an in vitro method for measuring such levels, or a device adapted to the same, and
b. processing the levels determined in step a. by comparing them to a control, and
c. determining through a predetermined decision rule, especially a decision rule associated with determined sensitivity and/or specificity associated with the investigated biomarkers, in particular if the levels of at least two biomarkers compared in step b. deviate from their controls, whether the levels received in step a. make that the human patient from which they have been measured, especially through an in vitro method for measuring the levels defined in in step a., has lesions rendering said patient at risk of a gastric cancer condition, and/or needs further medical test in relation thereto, especially clinical investigation.

The determination of step c. can be carried out according to any rule disclosed in present disclosure, according to the biomarkers that have been selected for implementation. For example, Table 2 herein provides an exemplary list of interesting decision rules. Decision rules can also derive from cut-off values determined for sensitivity and specificity values determined to be acceptable for the test to be carried out. Present description can be relied upon in light of all the examples of biomarkers lists associated with sensitivity, specificity and AUC values. The skilled person can readily implement a method carried out by a computer embedding the discussed step c. based on any data described in present disclosure. It is to be understood that the method carried out by a computer discussed herein can be implemented for any embodiment described herein in the context of an in vitro method of determining whether a human patient has lesions rendering said patient at risk of a gastric cancer condition and/or needs further medical test in relation thereto, in particular as defined in the claims or any embodiment of present description.

The invention also relates to a data processing apparatus comprising means for carrying out the method carried out by a computer discussed above, especially steps b. and/or c. of said method, or comprising a processor adapted to (or configured to) perform the said method, especially a processor adapted to (or configured to) perform steps b. and/or c. of the said method.

According to a particular embodiment, such a data processing apparatus comprises:
an input interface to receive the levels of the at least two biomarkers (or any combination of biomarkers as described in present description) defined in step a. of the method carried out by a computer discussed above,
a memory for storing at least instructions of a computer program comprising instructions which, when the program is executed by a computer or processor, cause the computer to carry out the method carried out by a computer discussed above, optionally a memory for storing control data and decision rules,
a processor accessing to the memory for reading the aforesaid instructions and executing the method carried out by a computer discussed above,
an output interface to provide at least the determination of whether the levels of the at least two biomarkers defined in step a. of the method carried out by a computer discussed above (or any combination of biomarkers as described in present description) make that the human patient from which they have been measured has lesions rendering said patient at risk of a gastric cancer condition, and/or needs further medical test in relation thereto, especially clinical investigation.

The invention also relates to a computer program (or computer product) comprising instructions which, when the program is executed by a computer or processor, cause the computer to carry out the method carried out by a computer discussed above.

The invention also relates to a computer-readable medium, in particular a computer-readable non-transient recording medium, having stored thereon the computer program (or computer product) discussed above, especially to implement the method carried out by a computer discussed above, when the computer program (or computer product) is executed by a computer or processor.

The present invention is a basis for a non-invasive test carried out in particular on a biological sample previously obtained from an individual, which may be a patient. By non-invasive test, it is meant that the method of the invention is in particular an in vitro method. Said method does not need the presence of a medical practitioner for its implementation. According to the invention, several biomarker(s) are proposed for an early detection of gastric carcinogenesis, in particular an early detection of the presence of gastric lesions involved or at the basis of a gastric carcinogenesis process, e.g., at an AG/P stage or condition as described herein. The present invention may be especially suitable for prevention purposes, but also: 1/ for monitoring the progression of a gastric carcinogenesis process, in an individual subjected or not to an on-going treatment for the condition he/she suffers, and/or 2/ monitoring a shift from a pre-neoplastic condition to a neoplastic condition, and/or 3/ as a follow-up after a cure to screen for a recurrence of the disease. For this purpose, the method of the invention simply relies on the detection and/or monitoring of physiological parameter(s) of a patient.

The method of the invention may be used on patients under treatment, such as chemotherapy treatment and/or radiations treatment, as an indicator of treatment efficiency, disease stage, and disease development.

The term “comprising” as used herein, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the application.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference.

The features described here-above and other features of the invention will be apparent when reading the examples and the figures, which illustrate the experiments conducted by the inventors, in complement to the features and definitions given in the present description. The following examples are offered by way of illustration. The examples are however not limitative with respect to the described invention.

### LEGEND OF THE FIGURES

FIG. 1. Plasmatic level of candidate biomarkers measured on all the samples of the cohort, A) mtDNA measured by qPCR on DNA isolated from circulating leukocytes; C) IL-8, E) TNF-α, G) IL-17, I) USF1 and K) USF2 measured using commercial ELISA assay as described in the methods section. Distribution of candidate biomarkers plasmatic level in the different groups of patients, H, NAG, AG/P, GC according to a determined cut-off value: B) mtDNA, D) IL-8, F) TNF-α, H) IL-17, J) USF1, L) USF2.

FIG. 2. Plasmatic level of candidate biomarkers measured on all the samples of the cohort, A) LEP, C) HP, E) SELE, G) MSLN, measured using commercial ELISA assays as described in the methods section. Distribution of candidate biomarkers plasmatic level in the different groups of patients, H, NAG, AG/P, GC according to a determined cut-off value: B) LEP, D) HP, F) SELE, H) MSLN

FIG. 3. Diagnostic accuracies of biomarker candidates as determined by ROC curve analysis (True Positive Rate (TPR) in function of False Positive Rate (FPR)) and AUC values. ROC curves have been obtained using decision rules “if the biomarker quantity is superior (or, depending upon the configuration, inferior, or superior or equal, or inferior or equal) to x, then the patients is H or NAG or AG/P or GC”, where x is a cut off value. Optimal cut off values and AUC criteria are displayed in the top left of each plot. Optimal cut off values have been determined using a compromise between TPR and FPR. Corresponding table for FIG. 3 is shown below:

| | | H | NAG | AG/P | GC |
|---|---|---|---|---|---|
| MtDNA | AUC | 0.3618 | 0.4956 | 0.7089 | 0.5382 |
| | Opt. cut | 6.55 | 4.76 | 6.24 | 4.6 |
| IL-8 | AUC | 0.2308 | 0.4771 | 0.4237 | 0.7744 |
| | Opt. cut | 4 | 16.0493 | 35.3086 | 29.3827 |
| IL-17 | AUC | 0 | 0.7513 | 0.7189 | 0.6675 |
| | Opt. cut | Inf | 78 | 89.1428 | 50.2 |
| TNF-alpha | AUC | 0.2045 | 0.6487 | 0.5776 | 0.6763 |
| | Opt. cut | 191.6 | 112.11 | 101.03 | 85.64 |
| USF1 | AUC | 0.3706 | 0.4279 | 0.4338 | 0.6761 |
| | Opt. cut | 119.23 | 171.5384 | 173.0769 | 96.15 |
| USF2 | AUC | 0.3704 | 0.5498 | 0.6089 | 0.5319 |
| | Opt. cut | 55 | 15.96 | 36.1538 | 40.19 |
| SELE | AUC | 0.2434 | 0.5483 | 0.578 | 0.6341 |
| | Opt. cut | 11.44 | 9.05 | 9.705 | 10.206 |
| MSLN | AUC | 0.287 | 0.5775 | 0.7433 | 0.5252 |
| | Opt. Cut | 13.42 | 8.4 | 10.83 | 9.95 |
| HP | AUC | 0.3771 | 0.4256 | 0.4687 | 0.6622 |
| | Opt. cut | 0.91 | 1.05 | 1.05 | 1.23 |
| LEP | AUC | 0.566 | 0.6426 | 0.8865 | 0.1898 |
| | Opt. cut | 4.2699 | 5.94 | 7.08 | 1.8479 |

FIG. 4. Diagnostic accuracies of biomarker candidates as determined by ROC curve analysis (True Positive Rate (TPR) in function of False Positive Rate (FPR)) and AUC values. ROC curves have been obtained using decision rules “if the biomarker quantity is inferior (or, depending upon the configuration, superior, or superior or equal, or inferior or equal) to x, then the patients is H or NAG or AG/P or GC”, where x is a cut off value. Optimal cut off values and AUC criteria are displayed in the top left of each plot. Optimal cut off values have been determined using a compromise between TPR and FPR. Corresponding table for FIG. 4 is shown below:

| | | H | NAG | AG/P | GC |
|---|---|---|---|---|---|
| MtDNA | AUC | 0.6381 | 0.5043 | 0.291 | 0.4617 |
| | Opt. cut | 4.57 | 6.05 | 10.23 | 6.67 |
| IL-8 | AUC | 0.7691 | 0.5228 | 0.5762 | 0.2255 |
| | Opt. cut | 17.7777 | 26 | 10.4 | 82.2222 |
| IL-17 | AUC | 1 | 0.2486 | 0.281 | 0.3324 |
| | Opt. cut | 41.18 | 89.1428 | 64 | 78 |
| TNF-alpha | AUC | 0.7954 | 0.3512 | 0.4223 | 0.3236 |
| | Opt. cut | 73.85 | 130.26 | 121.05 | 146.3 |
| USF1 | AUC | 0.6293 | 0.572 | 0.5661 | 0.3228 |
| | Opt. cut | 96.15 | 82.3076 | 100 | 171.5384 |
| USF2 | AUC | 0.6295 | 0.4501 | 0.391 | 0.468 |
| | Opt. cut | 14.42 | 38.0769 | 49.81 | 29.23 |
| SELE | AUC | 0.7565 | 0.4516 | 0.4219 | 0.3658 |
| | Opt. cut | 7.1 | 10.51 | 10.323 | 9.64 |
| MSLN | AUC | 0.7129 | 0.4224 | 0.2586 | 0.4747 |
| | Opt. Cut | 8.33 | 11.01 | 20.21 | 8.7 |
| HP | AUC | 0.6228 | 0.5743 | 0.5312 | 0.3377 |
| | Opt. cut | 1.08 | 1.1299 | 1.1 | 0.89 |
| LEP | AUC | 0.4339 | 0.3573 | 0.1134 | 0.801 |
| | Opt. cut | 5.94 | 2.3514 | 18.04 | 4.1 |

FIG. 5. Functional association network between biomarker candidates identified by Proteome profiler arrays (STRING analysis: https://string-db.org/) and their related cellular function. Known Interactions: from curated databases ( ) experimentally determined ( [symbol] ) predicted Interactions: co-expression ( [symbol] ) others: textmining ( [symbol] )

FIG. 6. Correlation matrix, hierarchical clustering and PLS-DA and sparse PLS-DA analysis. A) Pairwise correlation matrix represents the Pearson correlation coefficients between each pair of samples computed using all complete pairs of intensity values measured in these samples. B) Hierarchical cluster analysis conducted as indicated in the methods section. C) Partial Least Square—Discriminant Analysis (PLS-DA) was used to investigate the proteomic differences between four patient groups (H, NAG, AG/P, GC). PLS-DA plot displayed a good separation between healthy patients and other pathologies. D) The sparse PLS-DA method selects a set of 85 potential biomarkers, allowing to clearly distinguish H subjects and GC patients but failed to separate NAG and AG/P patients.

FIG. 7. Heatmap displaying the deviations from their mean intensity level of the most relevant biomarker candidates identified by MS. For most of the factors a common profile is mainly observed for NAG and AG/P and a distinct profile characterizes GC compared to the other groups. This Figure encompasses information in color: color figures have been submitted to the Office at the time of the filing, which can be relied upon.

FIG. 8. Analysis and prediction for candidate biomarkers measured by ELISA assay. A) Repartition of AUC values for all combinations of 2 variables for each group of patients Healthy (H), non-atrophic gastritis (NAG), Pre-neoplasia (AG/P) and cancer (GC). B) Number of times one candidate appears in the model giving the best AUC criteria for 2 variables.

FIG. 9. Residual deviance of the model to measure its capacity to predict all pathologies simultaneously. Ten best combinations of 2 biomarkers, highlighting the ability of the combination of IL-17 and LEP to predict H and patients (upper part). Ten best combinations of 3 biomarkers showing the association TNF-α, IL-17 and LEP to separate H from patients, mainly GC (lower part).

Corresponding 10 best combinations of 2 biomarkers and 3 biomarkers shown in FIG. 9 are reproduced below:

### 10 Best Combinations of 2 Biomarkers (in Term of Residual Deviance) (Upper Part)

1) IL-17 and LEP: 151.23
2) TNF-α and IL-17: 154.25
3) IL-17 and USF2: 162.58
4) Sex and IL-17: 176.26
5) IL-17 and MSLN: 190.51
6) MtDNA and IL-17: 191.22
7) Hp status and 1L-17: 191.22
8) IL-17 and USF1: 196.64
9) IL-8 and IL-17: 198.49
10) IL-17 and HP: 198.81

### 10 Best Combinations of 3 Biomarkers (in Term of Residual Deviance) (Lower Part)

1) TNF-α, IL-17 and LEP: 124.88
2) IL-17, USF2 and LEP: 135.12
3) MtDNA, IL-17 and LEP: 135.28
4) IL-17, HP and LEP: 139.20
5) Sex, TNF-α and IL-17: 140.53
6) TNF-α, IL-17 and USF2: 141.95
7) Sex, IL-17 and USF2: 142.34
8) IL-17, MSLN and LEP: 142.98
9) TNF-α, IL-17 and MSLN: 143.40
0 10) MtDNA, TNF-α and IL-17: 143.54

Of note, mentions of «Sex» and «Hp status» in this Figure and above refer to the fact that in the course of the experiments carried out, it also has been tested whether patient sex or Hp status, i.e., the fact that the patient is known to be positive to Helicobacter pylori infection, could have an incidence on each detection of each disease stage, according to the definitions provided herein.

FIG. 10. Results of the model estimation. A) Distribution of AUC criteria for 2 and 3 variables for non atrophic gastritis (NAG), pre-neoplasia (AG/P) and cancer (GC) groups. B) Number of times a biomarker candidate appears in the models giving the best AUC criteria

FIG. 11. Residual deviance of the model to measure its capacity to predict all pathologies simultaneously. Ten best combinations of 2 biomarkers, highlighting the ability of the combination of KIF20B with ARG1 or CPA4 to predict GC (upper part). Ten best combinations of 3 biomarkers showing a perfect classification for all groups with the association: KRT19, KIF20B and SPEN (lower part).

Corresponding 10 best combinations of 2 biomarkers and 3 biomarkers shown in FIG. 11 are reproduced below:

### 10 Best Combinations of 2 Biomarkers (in Term of Residual Deviance) (Upper Part)

1) ARG1 and KIF20B: 21.86
2) KIF20B and CPA4: 21.95
3) DSP and KIF208: 25.06
4) SPRR1A and S100A12: 25.17
5) S100A12 and CPA4: 25.36
6) MAN1A1 and SPRR1A: 25.55
7) ARG1 and S100A12: 25.61
8) S100A12 and MAN2A1: 25.87
9) KRT19 and S100A12: 25.95
10) CFP and CDSN: 26.04

### 10 Best Combinations of 3 Biomarkers (in Term of Residual Deviance) (Lower Part)

1) KRT19, KIF20B and SPEN: 0.47
2) ARG1, KIF20B and SPEN: 2.37
3) DSP, KIF20B and SPEN: 2.38
4) HAL, KIF20B and SPEN: 3.50
5) C7, KIF20B and SPEN: 5.09
6) JUP, KIF20B and SPEN: 5.79
7) KIF20B, SPEN and CPA4: 6.01
8) SPRR1A, KIF20B and SPEN: 6.24
9) MAN2A1, KIF20B and SPEN: 6.68
10) KRT14, KIF20B and SPEN: 8.49

FIG. 12. Biomarkers signature giving the most perfect predictions to identify the different stages of the GC process. As indicated IL-17 allows to distinguish between healthy and patients. Among patients the association of KIF20B, SPEN either with KRT19, ARG1, DSP1 or Hal gives a good prediction of pre-neoplasia (AG/P). In italics are also candidates to be considered among others, according to the corresponding AUC values observed for pre-neoplasia prediction. In addition LEP and S100A12 are pertinent for a good prediction of GC.

FIG. 13. Plasma level of biomarker candidates confirmed by ELISA. Violin plots representing the plasma levels of candidate biomarkers firstly identified either by MS, proteome profiler and confirmed by commercial ELISA or directly measured by ELISA. Quantifications were performed on all samples of the cohort. Statistical analysis using Mann-Whitney test significant for p<0.05. This Figure includes ELISA results for biomarkers EGFR and STAT3.

FIG. 14. STRING graphic representation of the functional network existing between the biomarker candidates. Among the 22 confirmed proteins, 14 and 2 are functionally connected. In addition, some proteins are part of a physical complex as: STAT3-EGFR-LEP-IL-8; MSLN-LBP and USF1-USF2. https://string-db.org.

FIG. 15. Best biomarker signatures to predict A. gastric preneoaplasia (SIG-AGP) and B. gastric cancer (SIG-GC). AUC increase with the length of the signature for both SIG-AGP and SIG-GC. It is associated with an increase of Sens for SIG-GC and Spec for SIG-AGP.

FIG. 16: Scheme of the use of a diagnostic test based on the detection of preneoplasia and GC lesions by SIG-AGP and SIG-GC signatures, respectively. Three different levels of use can be proposed: 1) screening of patients at risk of GC; 2) follow-up of the presence of preneoplasia to detect GC development at the earliest steps; 3) follow-up of GC patients after surgery and during/after chemotherapy to prevent recurrence of cancer. The legend of the diagram is as follows: 1. Testing a biological sample drawn from a patient or pool of patients, e.g., a blood or plasma sample, against the, e.g., SIG-AGP or SIG- GC signature(s) described herein, or any other combination of markers as described herein, 2. If the test of 1. is negative, no further action is to be envisioned, 3. If the test of 1. is positive for a risk of AG/P (SIG-AGP Positive), then proceeding further to 5., 4. If the test of 1. is positive for a risk of GC (SIG-GC Positive), then proceeding further to 5., 5. Further clinical investigation, for example, carrying out an endoscopy procedure on the patient, from which the assayed sample was drawn, 6. If step 5. concludes to the presence or risk of preneoplasia through the further clinical investigations of 5., then proceeding further to 8. as a patient follow-up procedure, 7. If step 5. concludes to the presence or risk of gastric cancer through the further clinical investigations of 5., then proceeding further to 9., 8. New/further testing of a biological sample drawn from the followed-up patient, e.g., a blood or plasma sample, against the, e.g., SIG-AGP or SIG-GC signature(s) described herein, or any other combination of markers as described herein. This new/further testing can be the beginning of a new round of testing starting from step 1. of present diagram, 9. Further medical action such as treatment and/or surgery of the followed-up patient, according to the data at disposal of the practitioner, 10. New/further testing of a biological sample drawn from the followed-up patient, e.g., a blood or plasma sample, against the, e.g., SIG-AGP or SIG-GC signature(s) described herein, or any other combination of markers as described herein. This new/further testing constitutes a patient follow-up procedure and can be the beginning of a new round of testing starting from step 1. of present diagram.

### EXAMPLES

### Methodology

### Study Population

The studied cohort is described in the Table 1. It includes 48 healthy (H) asymptomatic volunteers recruited at the clinical investigation and biomedical research support unit (ICAReB) at the Institut Pasteur. Each H samples were confirmed for their H. pylori-negative serology using a commercial Enzyme-linked Immunosorbent Assay (ELISA) (Serion ELISA Classic). Twenty-six non-atrophic gastritis (NAG), 38 atrophic gastritis/pre-neoplasia (AG/P) and 68 gastric cancer (GC) patients are included in the cohort. NAG and AG/P patients were diagnosed in the service of Hepato-Gastroenterology headed by Pr D. Lamarque (AP-HP, A. Paré hospital, Boulogne-Billancourt). GC patients were diagnosed in the service of Hepato-Gastroenterology and Digestive Oncology headed by Pr J. Taieb, AP-HP, HEGP, Paris. All patients were adults, not under anticancer treatment, not treated with antibiotics, bismuth compounds, proton pump inhibitors and non-steroidal anti-inflammatory drugs for at least the two preceding weeks. Diagnosis was based on endoscopic examination and histopathology analysis of the gastric biopsies. All patients were informed and asked to sign a consent letter. The study was approved by the Institut Pasteur translational research center (Ref protocol: 2013-29).

TABLE 1
Characteristics of the study population

| | Mean age | Sex Ratio | |
|---|---|---|---|
| | (range) | M/F | H. pylori positive |
| Healthy (n = 48) | 41 (21-70) | 0.4 | 0 |
| NAG (n = 26) | 59 (27-88) | 0.7 | 50% |
| AG/P (n = 38) | 68 (19-88) | 0.9 | 61% |
| GC (n = 68) | 61 (30-84) | 2 | 29% |
| Total n = 180 | | | |

### Collection of Clinical Samples and Histological Analysis

For each patient, 10 ml of blood were collected and gastric tissue specimens isolated. Gastric biopsies from both antrum and corpus were collected during gastric endoscopy. Biopsies were immersed in formalin and processed for haematoxilin-eosin (H&E) staining for histology analysis and diagnosis of gastric lesions. The presence of H. pylori was confirmed by Giemsa staining and serology. The H. pylori negative status of blood samples from healthy volunteers from ICAReB platform was verified by serology before to be included in the cohort.

Circulating Mitochondrial DNA (mtDNA) and Quantification

Peripheral blood (10 ml) is taken from each patient, and leucocytes isolated on Leucosep® tubes by pancoll gradient. DNA was prepared from isolated leucocytes using Qiamp DNA kits (Qiagen) and frozen at −80° C. until tested for mtDNA quantification. In parallel, plasmatic fractions are isolated and frozen at −20° C. until to be used. MtDNA levels were measured on DNA isolated from circulating leukocytes by quantitative Polymerase Chain Reaction (q-PCR) using a StepOne™ Plus Real-Time PCR system and FastStart Universal SYBR Green Master (Applied Biosystems) as previously described (6), using the 12S ribosomal RNA gene and the nuclear encoded 18S ribosomal RNA gene as endogenous reference. The relative mtDNA level was calculated using the delta Ct (ΔCt) of average Ct of nDNA and mtDNA (ΔCt=Ct_{nDNA}−Ct_{mtDNA}) as 2^{ΔCt} as already reported (7).

### Quantification of Plasmatic Levels of the Different Biomarker Candidates

Plasmatic levels of the different selected biomarker candidates were evaluated using commercial ELISA assays from Duo Set RD system. Interleukin-8 (IL-8) (Ref DY208); Interleukin-17 (IL-17) (Ref DY317); Tumor necrosis factor-α (TNF-α) (Ref DY210); Mesothelin (MSLN) (Ref DY3265); E-Selectin (SELE) (Ref DY724); Haptoglobin (HP) (Ref DY8465); Leptin (LEP) (Ref DY398) and Upstream stimulating factor 1 and 2 (USF1 Ref MBS9342772 and USF2 Ref MBS9321077; My Bio Source).

Plasmatic levels of factors related to Oncology pathways were screened by proteomic profiler analysis using the Human XL oncology array (Ref ARY029; R&D systems), respectively. This array consists in capture selected antibodies present on nitrocellulose membranes that were incubated with plasma samples mixed with a cocktail of biotinylated detection antibodies and revealed by streptavidin-horseradish peroxidase according to the supplier recommendations. For each pathway, 2 samples representatives of each group of patients NAG, AG/P and GC, also including healthy (H) subjects were analyzed.

### Large-Scale Screening of Plasma Biomarker Candidates by Mass Spectrometry-Based Proteomics (MS)

### Plasma Samples

Four groups of n=10 samples were considered in this pilot study, including H, NAG, AG/P and GC patients. Representative plasma samples of each group were selected in the same cohort from AP-HP hospitals, used for all the project.

### MARS Hu-14 Immunodepletion

Plasma samples were depleted using the MARS Hu-14 (5188-6560-Agilent) following the manufacturers protocol. Briefly, 300 μg of total proteins were diluted with buffer A and filtered at 0.22 μm. Each sample was loaded into the spin column and centrifuged at 100 g/l min/RT. After 5 min of incubation time, the non-depleted proteins were eluted with 2 rounds of 400 μL buffer A by a centrifugation at 100 g/2.5 min/RT. The 3 filtrates were combined and further precipitated with TCA 40% (vol:vol) overnight. Samples were washed 2 times with acetone and air-dried before in-solution digestion.

### In-Solution Digestion

Depleted samples were resuspended with 100 μL of a 8M Urea/100 mM NH₄HCO₃ denaturation buffer and reduced with 5 mM TCEP (646547—Sigma, St Louis, Missouri, USA) for 15 min followed by alkylation with iodoacetamide 20 mM (I114—Sigma, St Louis, Missouri, USA) for 30 min into the dark. Proteins were digested with rLys-C 0.5 μg (V1671—Promega, Madison, Wisconsin, USA) for 3 h/37° C. and then diluted 9 times for a subsequent digestion with Sequencing Grade Modified Trypsin 0.5 μg (V5111—Promega, Madison, Wisconsin, USA) overnight/37° C. The digestion was stop with 4% Formic acid (FA) and peptides were desalted with a reversed phase C18 Stage-Tips method (8). Peptides were eluted with 80% Acetonitrile (ACN)/0.1% FA. Finally, samples were dried in vacuum centrifuge and resuspended with 2% ACN/0.1% FA. For all samples, iRT peptides were spiked as recommended by Biognosys.

### Peptide Fractionation for Spectral Library

A “pool” sample composed of the 40 plasma samples was dedicated to obtain a spectral library for the data independent acquisition (DIA) approach. The “pool” sample was depleted and digested with the previous protocols and a peptide fractionation was done using poly(styrene-divinylbenzene) reverse phase sulfonate (SDB-RPS) Stage-Tips method as described in (8) (9). Briefly, 3 SDB-RPS Empore discs were stacked on a P200 tip and 7 serial elutions were applied as following: elution 1 (60 mM Ammonium formate (AmF)/20% ACN/0.5% FA), elution 2 (80 mM AmF/30% ACN/0.5% FA), elution 3 (95 mM AmF/40% ACN/0.5% FA), elution 4 (110 mM AmF/50% ACN/0.5% FA), elution 5 (130 mM AmF/60% ACN/0.5% FA), elution 6 (150 mM AmF/70% ACN/0.5% FA) and elution 7 (80% ACN/5% ammonium hydroxide). All fractions were dried and resuspended with 2% ACN/0.1% FA before injection. For all fractions, iRT peptides were spiked as recommended by Biognosys.

### Mass Spectrometry Analysis

Data Dependent Acquisitions (DDA) for the spectral library: A nanochromatographic system (Proxeon EASY-nLC 1200—Thermo Fisher Scientific, Waltham, Massachusetts, USA) was coupled on-line to a Q Exactive™ HF Mass Spectrometer (Thermo Fisher Scientific) using an integrated column oven (PRSO-V1—Sonation GmbH, Biberach, Germany). For each sample, 1 μg of peptides was injected onto a 44 cm home-made C18 column (1.9 μm particles, 100 Å pore size, ReproSil-Pur Basic C18—Dr. Maisch GmbH, Ammerbuch-Entringen, Germany) after an equilibration step in 100% solvent A (H₂O, 0.1% FA). Peptides were eluted with a multi-step gradient from 2 to 7% solvent B (80% ACN, 0.1% FA) during 5 min, 7 to 23% solvent B during 70 min, 23 to 45% solvent B during 30 min and 45 to 95% solvent B during 5 min at a flow rate of 250 nL/min over 132 min. Column temperature was set to 60° C. Mass spectra were acquired using Xcalibur software using a data-dependent Top 10 method with a survey scans (300-1700 m/z) at a resolution of 60,000 and a MS/MS scans (fixed first mass 100 m/z) at a resolution of 15,000. The AGC target and maximum injection time for the survey scans and the MS/MS scans were set to 3.0E+06, 100 ms and 1.0E+05, 45 ms respectively. The isolation window was set to 1.6 m/z and normalized collision energy fixed to 28 for HCD fragmentation. We used a minimum AGC target of 2.0E+03 for an intensity threshold of 4.4E+04. Unassigned precursor ion charge states as well as 1, 7, 8 and >8 charged states were rejected and peptide match was disable. Exclude isotopes was enabled and selected ions were dynamically excluded for 45 seconds.
Data Independent Acquisitions (DIA) for plasma samples: Mass spectra were acquired in data-independent acquisition mode with the XCalibur software using the same nanochromatographic system coupled on-line to a Q Exactive™ HF Mass Spectrometer. For each sample, 1 μg of peptides was injected onto a 50 cm home-made C18 column (1.9 μm particles, 100 A pore size, ReproSil-Pur Basic C18—Dr. Maisch GmbH, Ammerbuch-Entringen, Germany) after an equilibration step in 100% solvent A (H₂O, 0.1% FA). Peptides were eluted with the same multi-step gradient than the spectral library. Each cycle was built up as follows: one full MS scan at resolution 60 000 (scan range between 349 and 1214 m/z), AGC was set at 3.0E+06 and maximum injection time was set at 60 ms. All MS1 was followed by 36 isolation windows of 25 m/z, covering the MS1 range. The AGC target was 2.0E+05 with an automatic maximum injection time and NCE was set to 28. All acquisitions were done in positive and profile mode.

### Data Processing for Protein Identification and Quantification

Building of the spectral library: Raw data were analyzed using MaxQuant software version 1.5.0.30 (10) using the Andromeda search engine (11). The MS/MS spectra were searched against the Human SwissProt database (20,203 entries the Dec. 4, 2018). Variable modifications (methionine oxidation and N-terminal acetylation) and fixed modification (cysteine carbamidomethylation) were set for the search and trypsin with a maximum of two missed cleavages was chosen for searching. The minimum peptide length was set to 7 amino acids and the false discovery rate (FDR) for peptide and protein identification was set to 0.01. The main search peptide tolerance was set to 4.5 ppm and to 20 ppm for the MS/MS match tolerance. Second peptides were enabled to identify co-fragmentation events.
Data analysis for DIA method: DIA experiments were analyzed using Spectronaut X (v. 13.2.190705.43655 Biognosys AG). Dynamic mass tolerance at the MS1 and MS2 levels was employed. The XIC RT Extraction Window was set to dynamic with a correction factor of 1. Calibration mode was set to automatic with nonlinear iRT calibration and precision iRT enabled. Decoys were generated using the mutated method and a dynamic limit. P-value estimation was performed using a kernel density estimator. Interference correction was enabled with no proteotypicity filter. Major grouping was by Protein-Group ID, and minor grouping was by stripped sequence. The major group quantity was mean peptide quantity. The major group top N was enabled with a minimum of 1 and a maximum of 3. Minor group quantity was mean precursor quantity. The minor group top N was enabled with a minimum of 1 and a maximum of 3. The quantity MSLevel was MS2, and quantity type was area. Q value was used for data filtering. Cross run normalization was enabled with Q value sparse for the row selection and local normalization for the strategy. The default labelling type was label-free with no profiling strategy and unify peptide peaks not enabled. The protein inference workflow was set to automatic.

The mass spectrometry proteomics data will be deposited to the ProteomeXchange Consortium via the PRIDE partner repository (12).

### Statistical Analysis

For each group of patients, the plasmatic levels for each biomarker candidates were first statistically analyzed using Mann-Whitney test. Results were considered significant if P<0.05.

### Determination of False Positive Rates (FPR), True Positive Rates (TPR), ROC Curves, and Area Under the Curve (AUC)

For a potential biomarker or a combination of biomarkers, a decision rule can be deduced to predict the stage of the disease (healthy, gastritis, pre-neoplasia or cancer). Therefore, the False Positive Rate (FPR) and True Positive Rate (TPR) are deduced from this decision rule by:

FPR=N/W

TPR=M/C

where N=Number of patients with an incorrectly predicted decision (ex.: number of non-cancer patients with a predicted cancer); W=Number of patients not checking the decision in reality (ex.: number of non-cancer patients); M=Number of patients with a correctly predicted decision (ex.: number of cancer patients with a predicted cancer); C=Number of patients checking the decision in reality (ex.: number of patients with cancer).

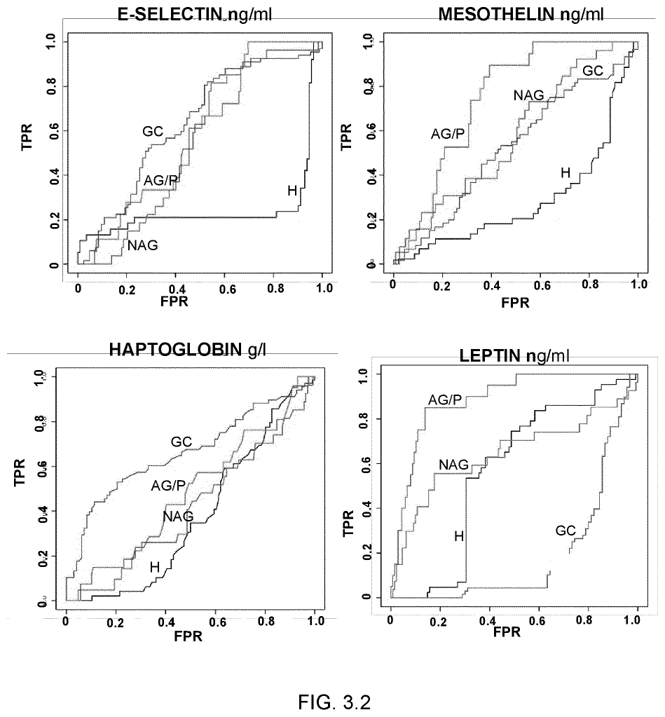

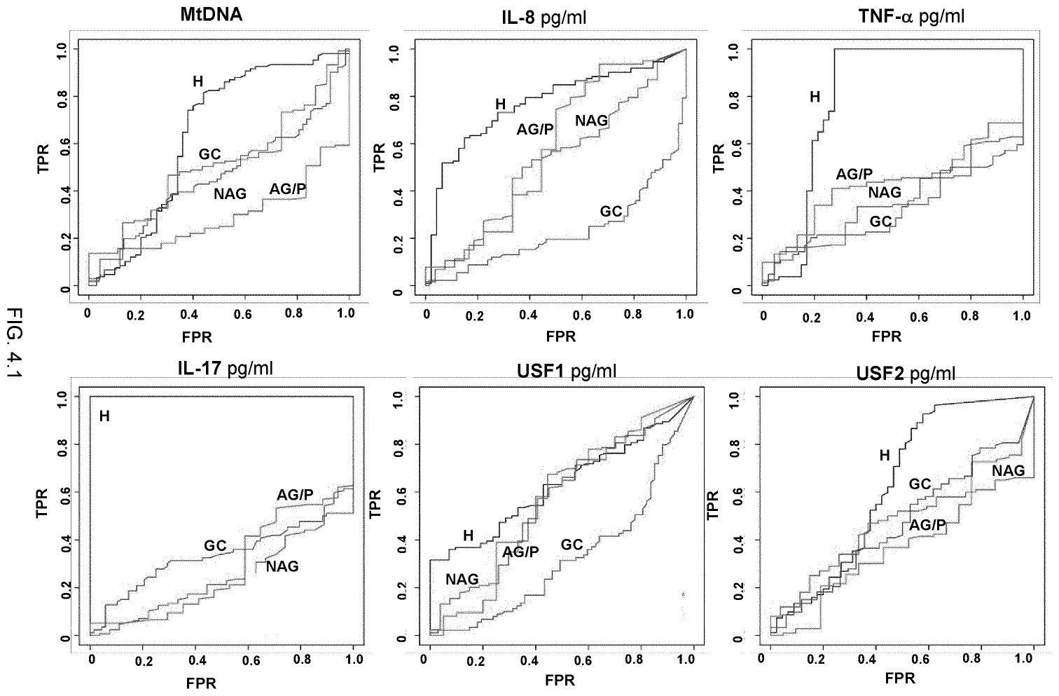

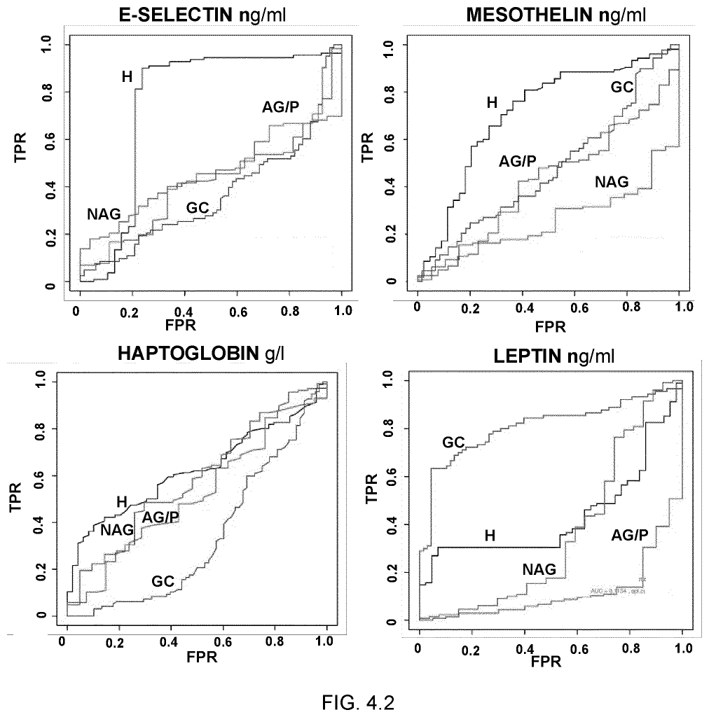

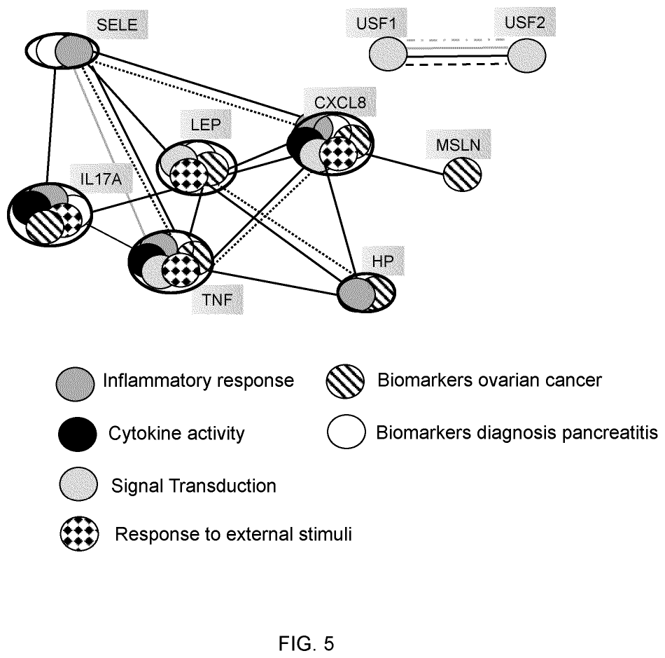

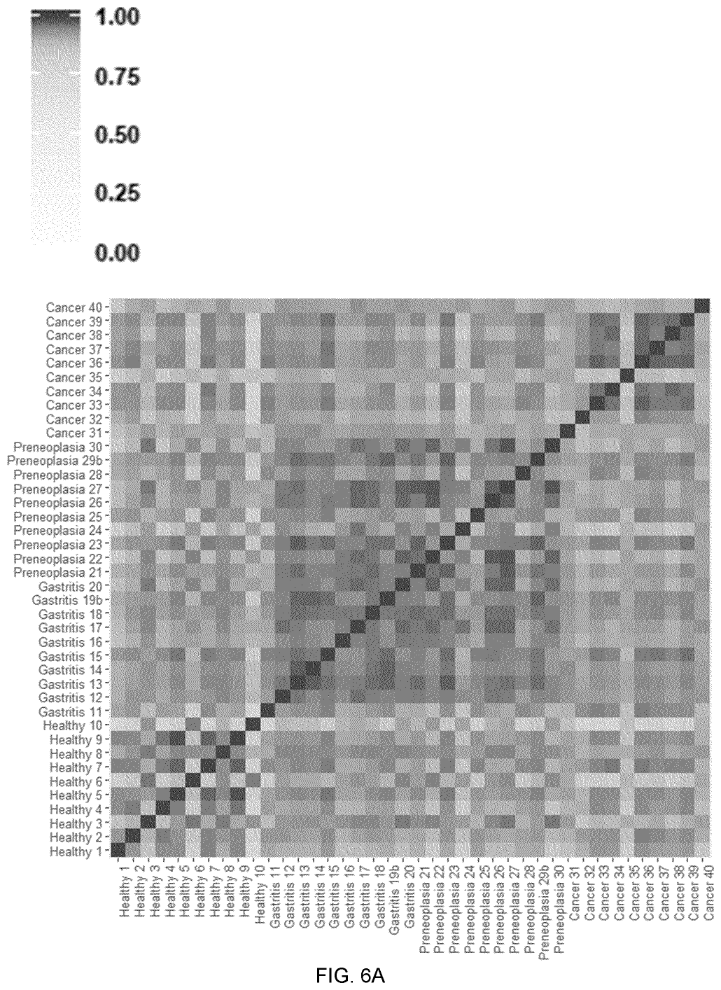

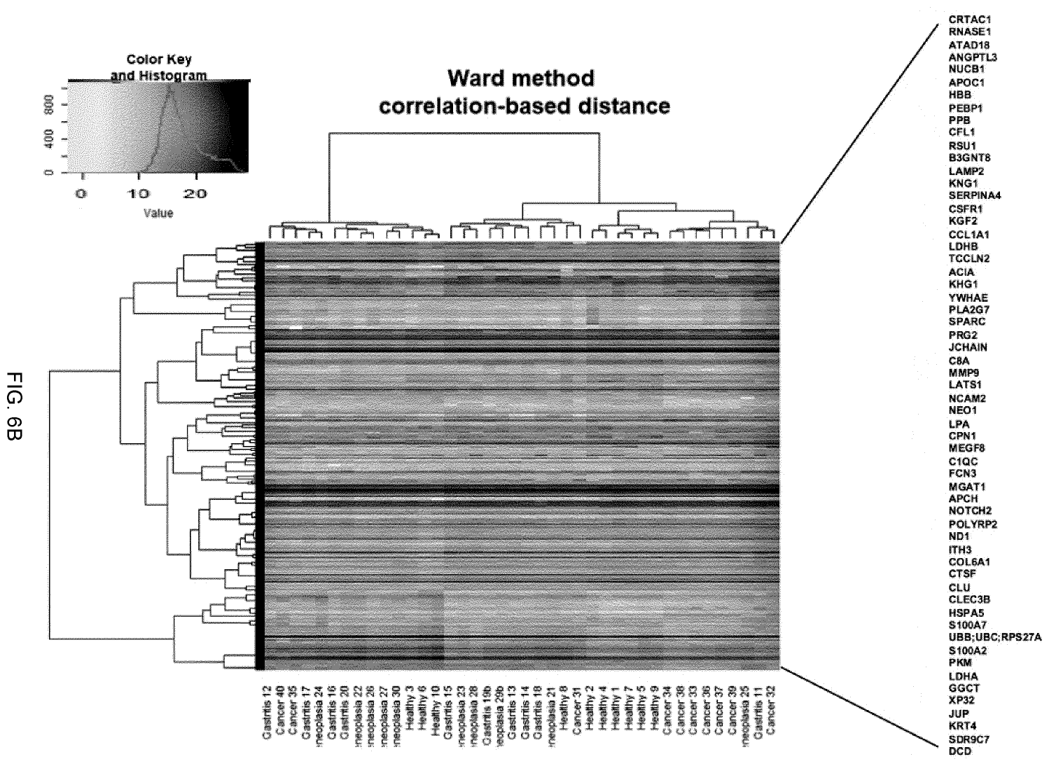

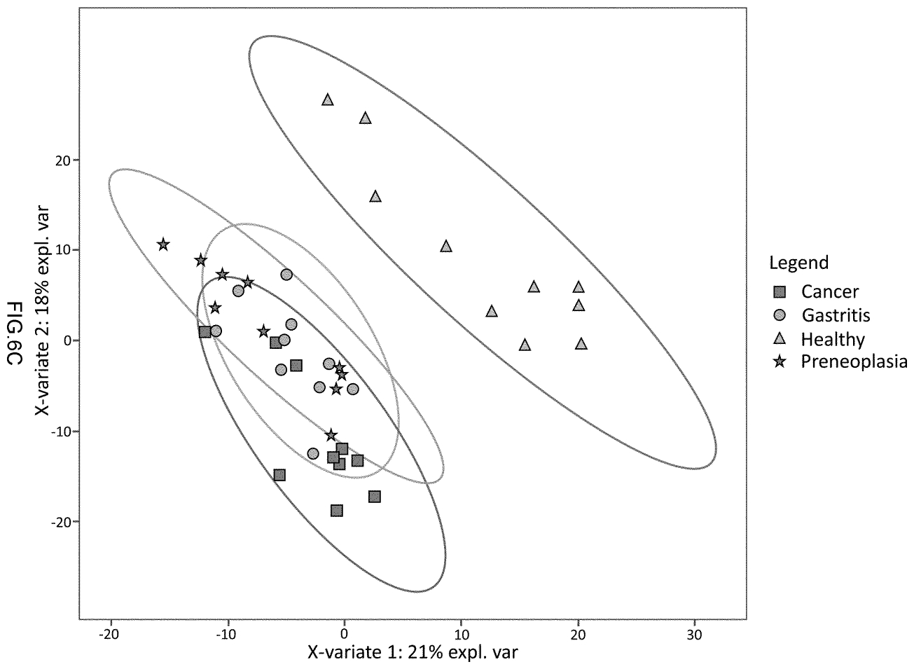

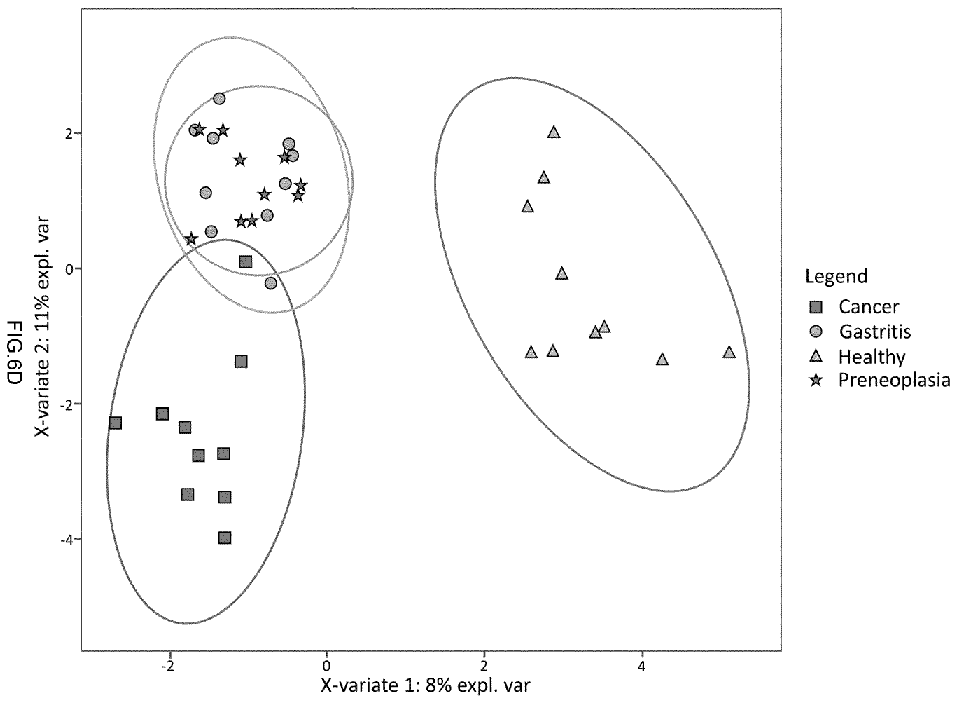

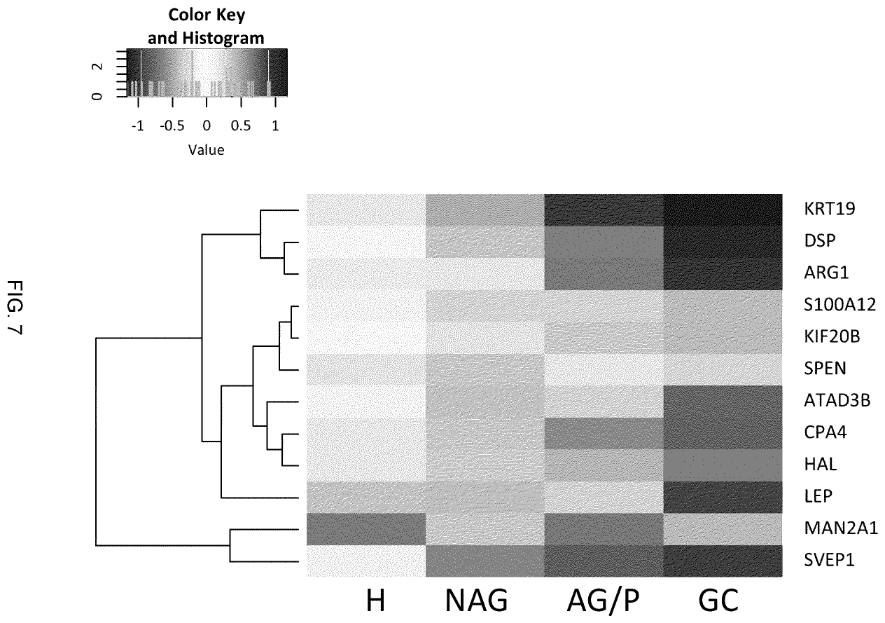

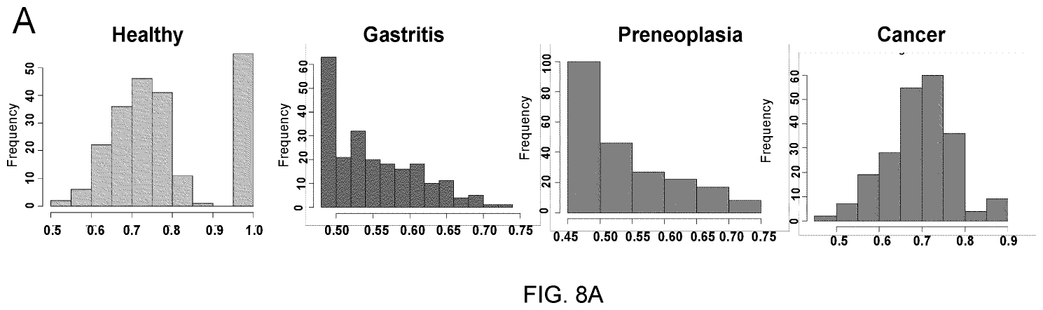

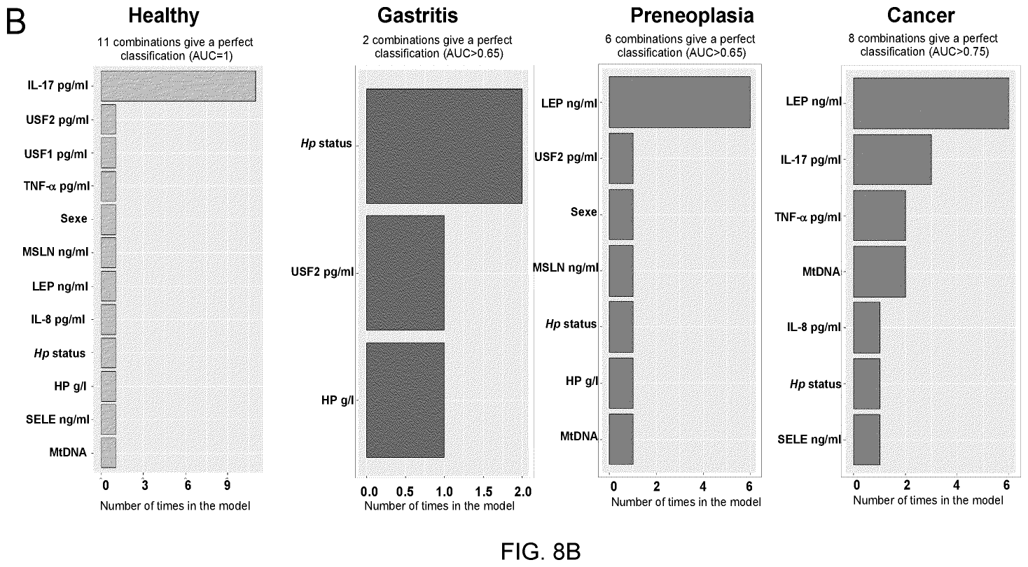

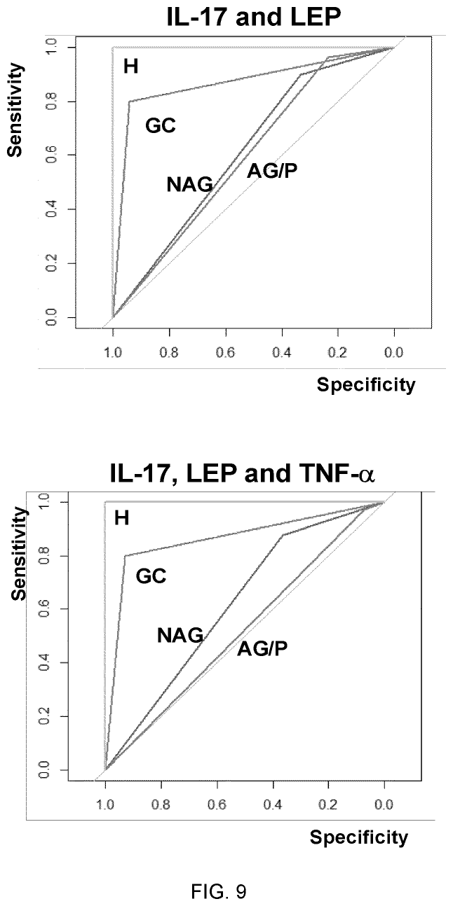

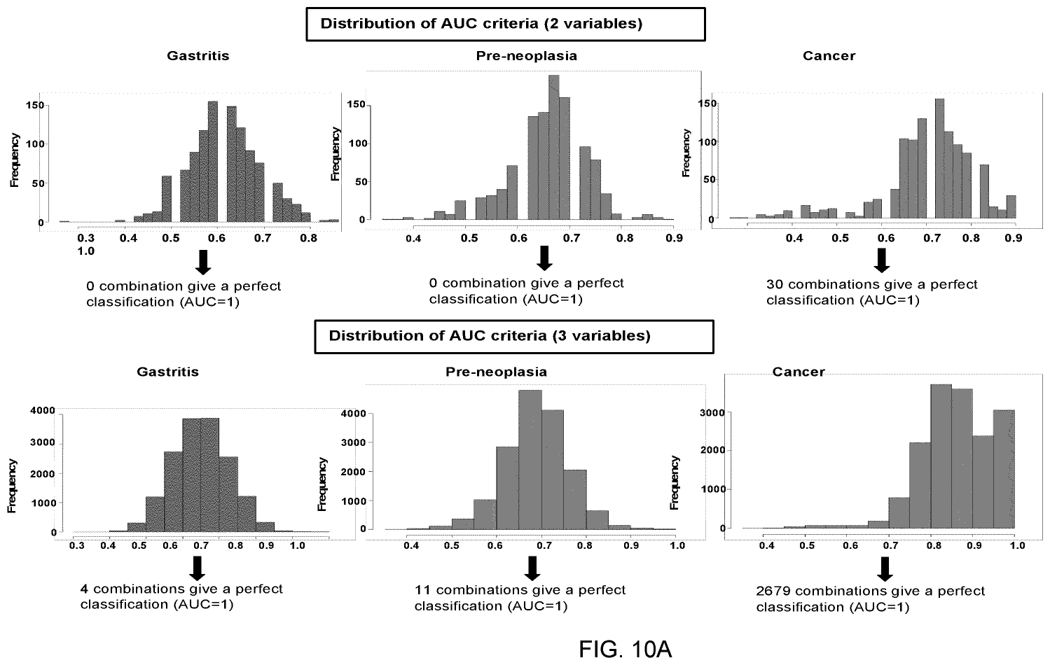

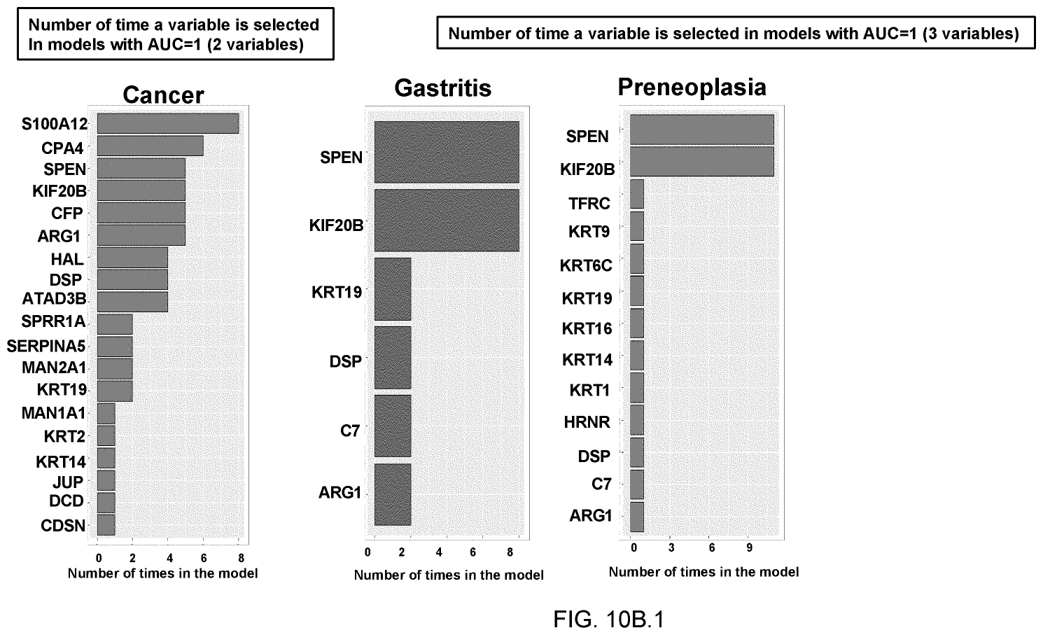

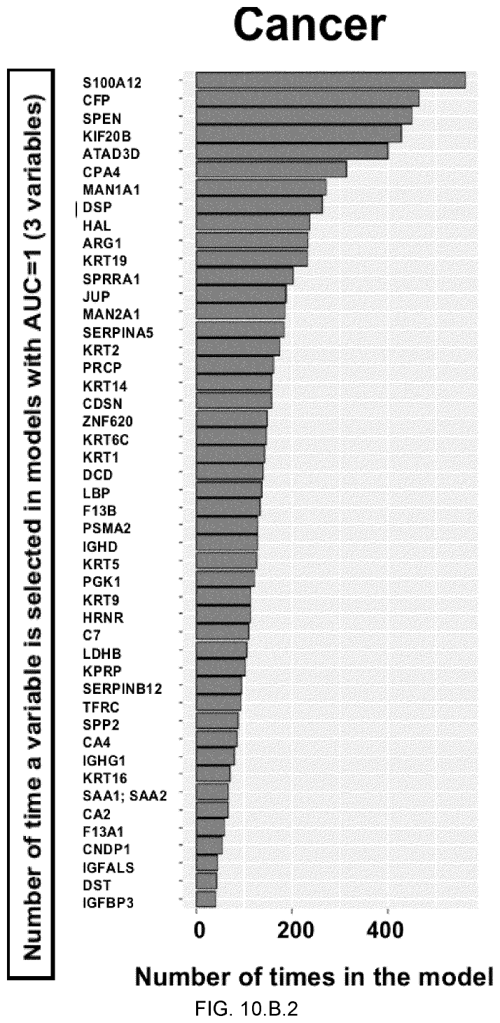

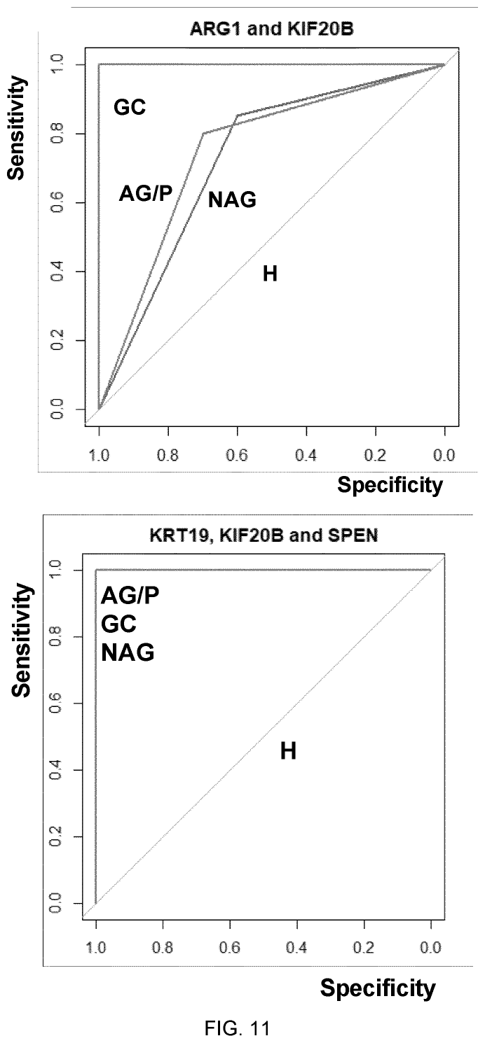

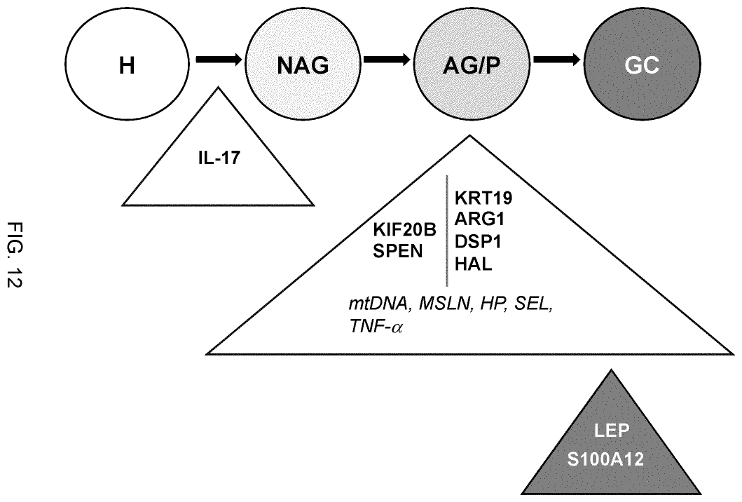

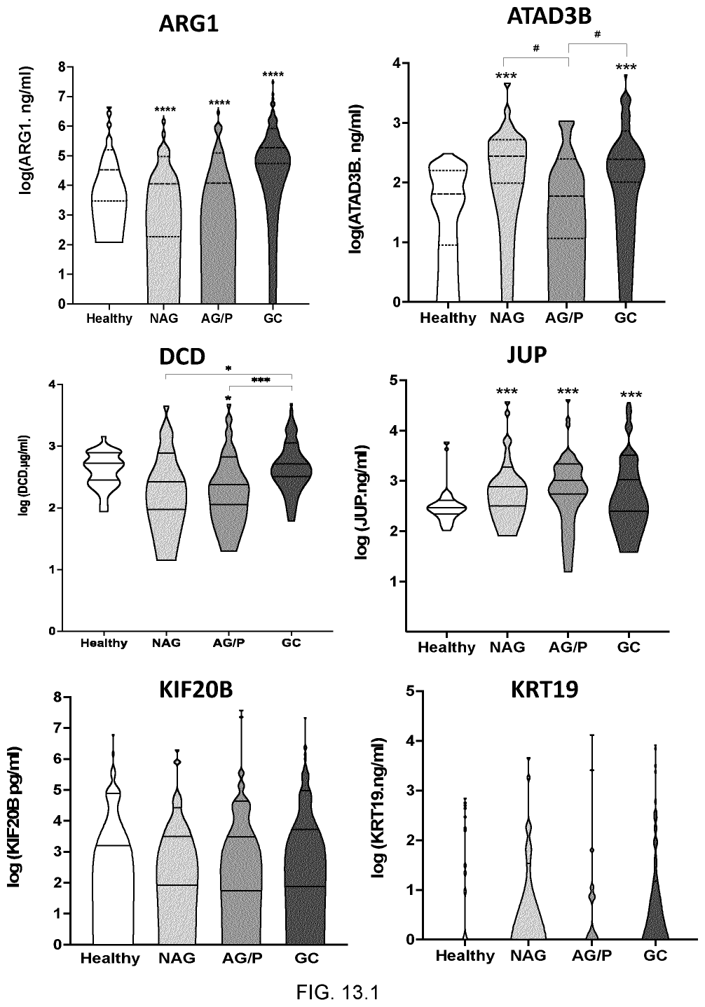

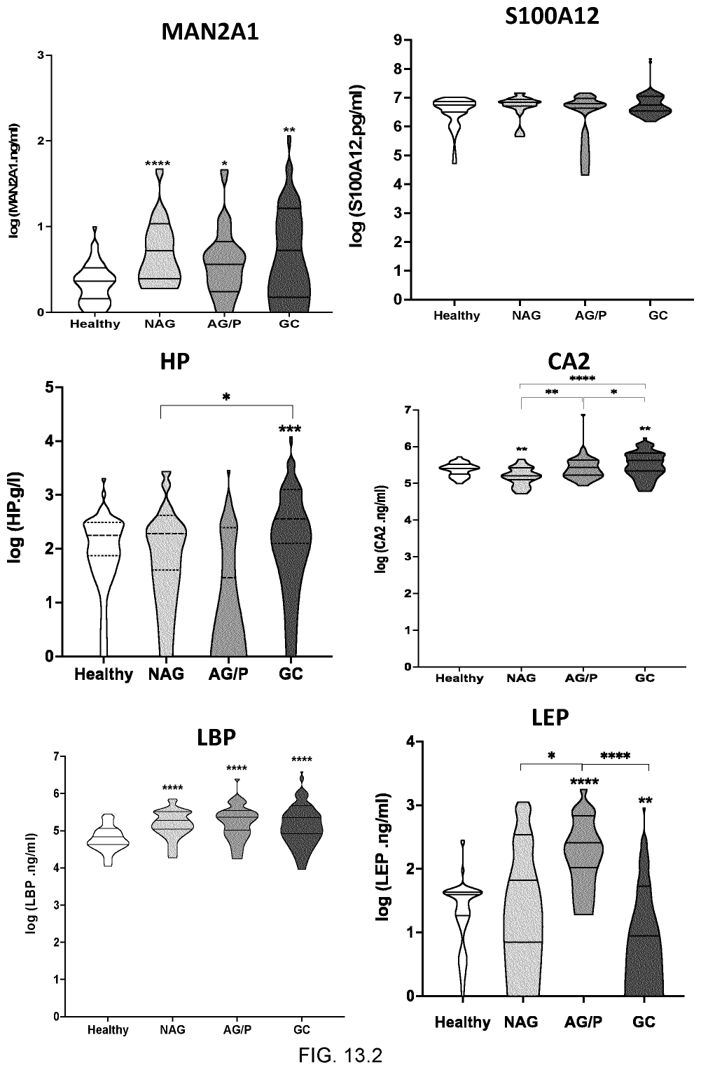

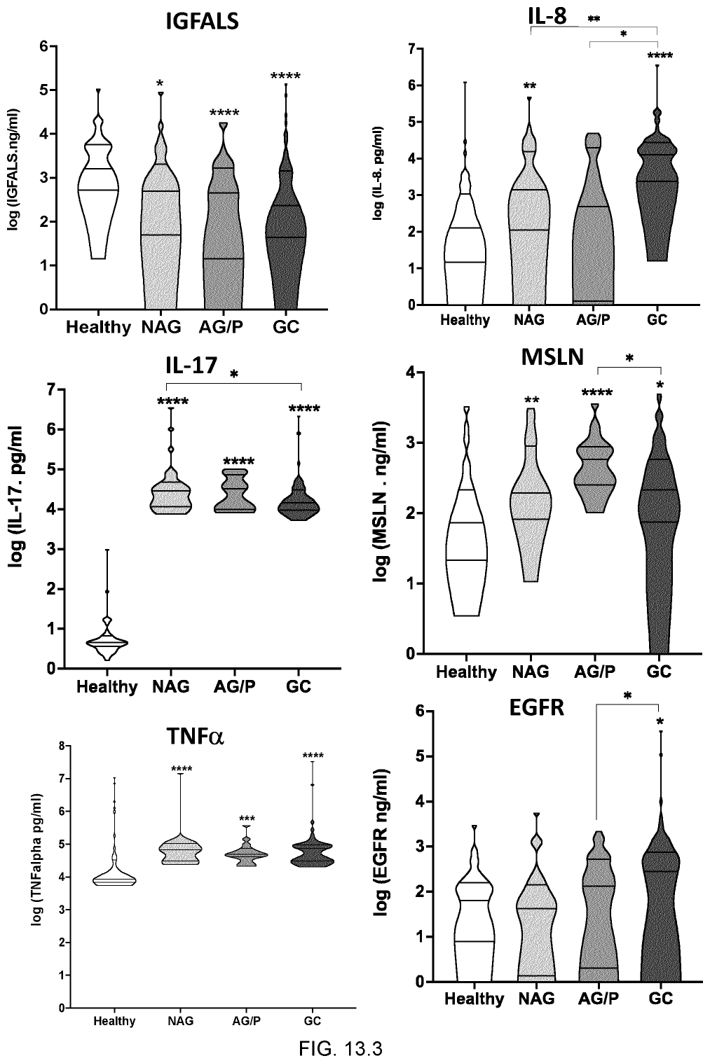

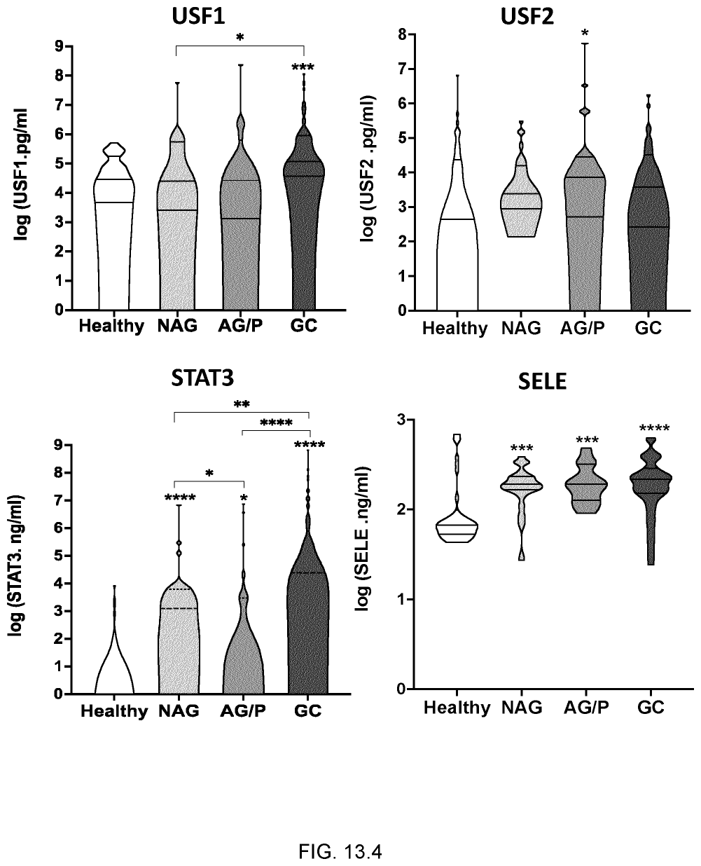

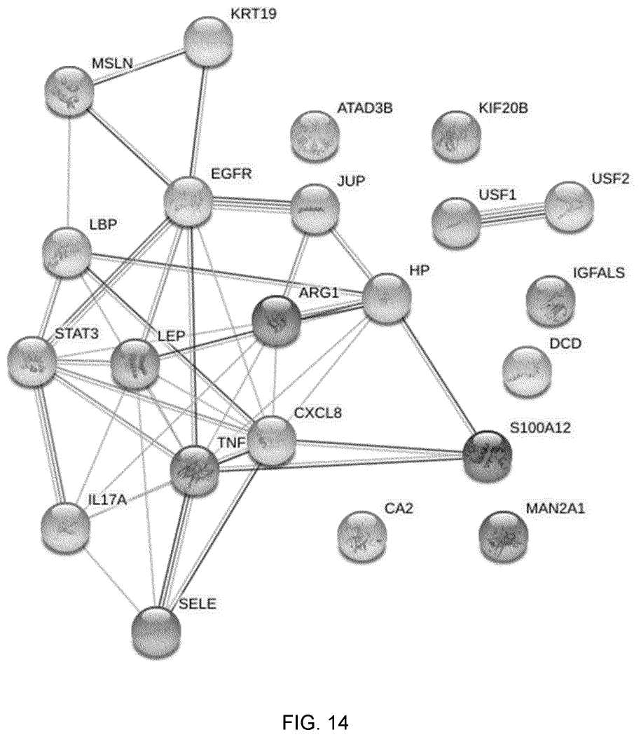

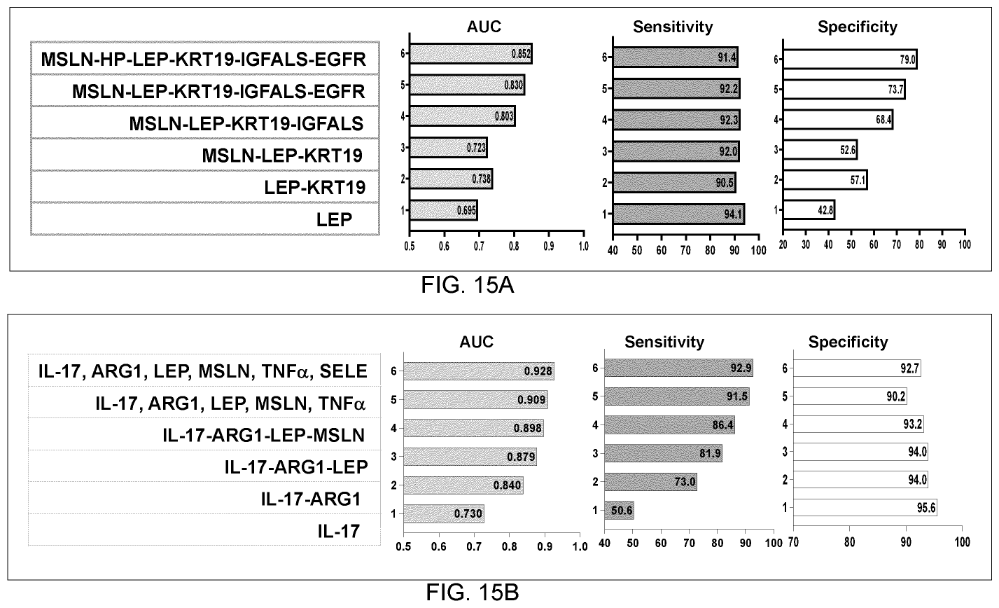

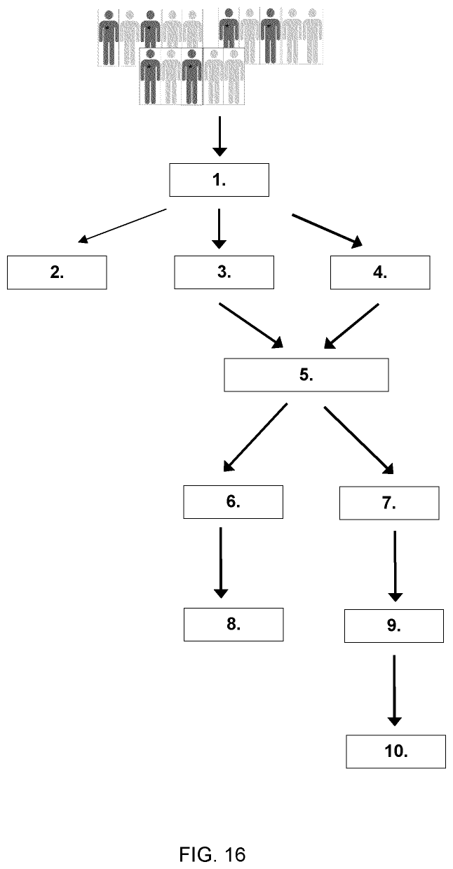

When using a single potential biomarker, a stage of the disease can be predicted using a threshold on the concentration of the potential biomarker. So, a FPR and a TPR are computed for each value of the threshold (Table 2). By varying the values of the threshold, a receiver operating characteristic (ROC) curve is determined with the FPRs on the x-axis and the TPRs on the y-axis (FIGS. 3 and 4). The area under the ROC curve is the AUC criterion. If AUC=0.5, then the biomarker cannot do better than a random selection of patients (whatever the chosen threshold), so the decision rule has no predictive power. Therefore, if AUC>0.5, the decision rule is better than a random selection (at least for one chosen threshold). Closer to 1 is the AUC, better is the decision rule (AUC=1 for an ideal decision rule). Similarly, for combinations of potential biomarkers, a diagnostic model is estimated to predict each stage of the disease, so that a FPR and a TPR are estimated, leading also to an AUC value (Table 4).

Exemplary literature regarding ROC curves: Delacour, H., et al., La Courbe ROC (receiver operating characteristic): principes et principales applications en biologie clinique, Annales de biologie clinique, 2005; 63 (2): 145-54.

### Multivariate Data Analysis and Selection of Potential Biomarkers

Correlation matrix and hierarchical clustering. Pairwise correlation analysis and hierarchical clustering have been performed to highlight similarities between plasma samples. The correlation matrix represents the Pearson correlation coefficients between each pair of samples computed using all complete pairs of intensity values measured in these samples. The hierarchical cluster analysis has been conducted via multiscale boostrap resampling (1000 bootstrap replications) with the Ward's method and a correlation-based distance measure thanks to the pvclust function of the R package pvclust, after log2 transformation of the intensities, imputation of the missing values with the impute.slsa function of the R package imp4p and a normalization using a sample-median centering method inside conditions.

Partial Last Square-Discriminant Analysis (PLS-DA) and sparse PLS-DA. PLS-DA was used to investigate the proteomic differences between four patient groups (H, NAG, AG/P, GC). PLS-DA and sparse PLS-DA was used using mixOmics R package.

### Prediction and Diagnostic Tests Using Combination of 2 or 3 Potential Biomarkers

To identify combinations of two or three potential biomarkers allowing to predict patient groups from measured intensities (Mass spectrometry analysis) or quantity (ELISA data), the following multinomial logistic regression model for a combination of k biomarkers was used:

$$ln ( \frac{P ( ind = Cancer )}{P ( ind = Healthy )} ) = {a}_{0} + {\sum }_{i = 1}^{k} {a}_{i} \times {v}_{i}$$

$$ln ( \frac{P ( ind = Preneoplasia )}{P ( ind = Healthy )} ) = {b}_{0} + {\sum }_{i = 1}^{k} {b}_{i} \times {v}_{i}$$

$$ln ( \frac{P ( ind = Gastritis )}{P ( ind = Healthy )} ) = {c}_{0} + {\sum }_{i = 1}^{k} {c}_{i} \times {v}_{i}$$

where:
k is the number of biomarkers used in the model.
vᵢ is the relative intensity value measured for a biomarker i. It corresponds to the concentration or intensity measured in a gastritis, pre-neoplasia or cancer sample divided by the average of concentration or intensity value that have been measured in healthy patients for this protein.

The estimation of this model has been evaluated using a residual deviance criterion, which is a goodness-of-fit statistic evaluating how well the model fits the observed stages of the disease (Table 4). Once the parameters aᵢ, bᵢ and cᵢ of the model are estimated, it is possible to compute a probability that the patient is affected by each stage of the disease with the following mathematical formula:

$$P ( ind = Healthy ) = \frac{1}{1 + S}$$

$$P ( ind = Cancer ) = \frac{exp ( {a}_{0} + {\sum }_{i = 1}^{k} {a}_{i} \times {v}_{i} )}{1 + S}$$

$$P ( ind = Preneoplasia ) = \frac{exp ( {b}_{0} + {\sum }_{i = 1}^{k} {b}_{i} \times {v}_{i} )}{1 + S}$$

$$P ( ind = Gastritis ) = \frac{exp ( {c}_{0} + {\sum }_{i = 1}^{k} {c}_{i} \times {v}_{i} )}{1 + S}$$

$$Where S = exp ( {a}_{0} + {\sum }_{i = 1}^{k} {a}_{i} \times {v}_{i} ) + exp ( {b}_{0} + {\sum }_{i = 1}^{k} {b}_{i} \times {v}_{i} ) + exp ( {c}_{0} + {\sum }_{i = 1}^{k} {c}_{i} \times {v}_{i} ) .$$

The highest probability among these four probabilities determines the most probable condition of the patient. This predicted state has been compared to the real state of the patient to determine AUC criteria for each stage of the disease (Table 4). Therefore, the estimated model can be used to diagnose a patient's disease state.

### Results

### Biomarker Candidates Selected According to Their Known Role in Carcinogenesis Process

### MtDNA

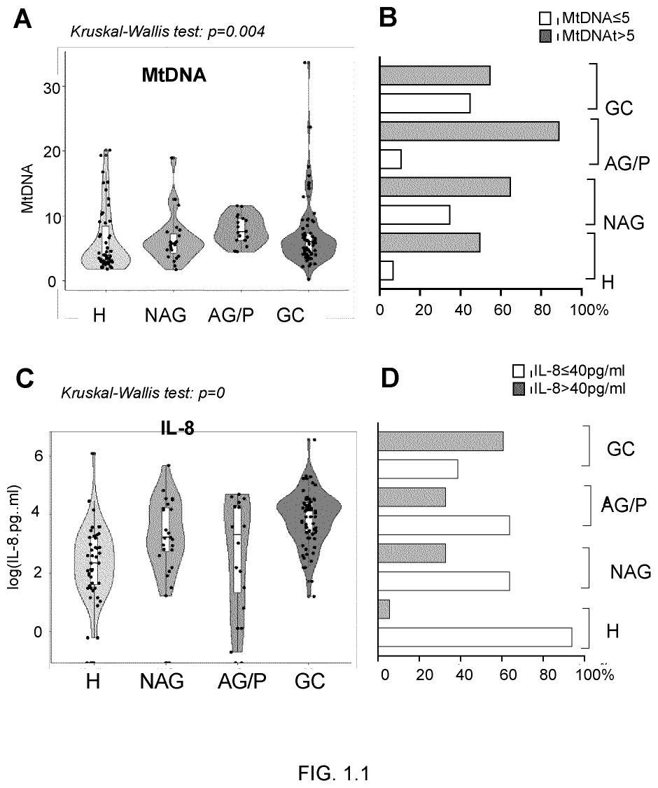

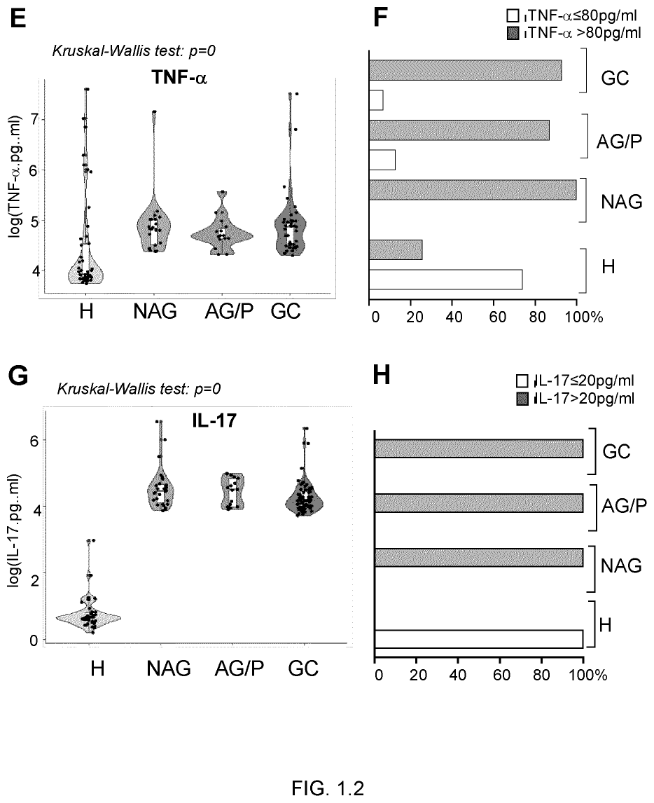

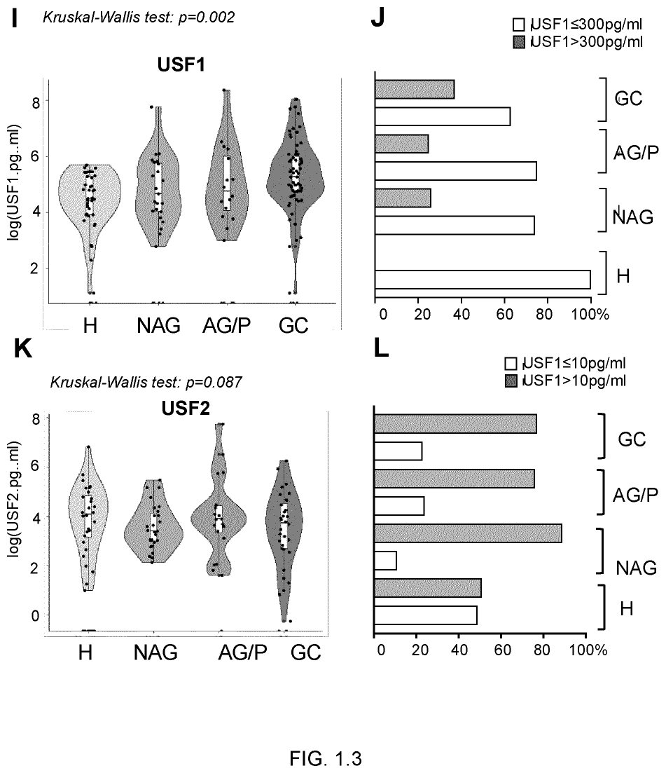

Both mtDNA mutations and variation of mtDNA content have been reported in different types of tumours. In a previous study performed on a cohort of Mexican patients, we reported higher levels of mtDNA (mtDNA>20) in circulating leukocytes mtDNA of GC patients, with a cut-off value of 8.23 (OR:3.93), that differentiates GC from H samples (6). These data were similar to mtDNA quantification performed with a Moroccan cohort of patients (ACIP 10-2015—Collaboration F. Maachi, Institut Pasteur Morocco). In the present study, mean mtDNA levels are 1.25 (P=0.04); 1.8 (P=0.0006) and 1.3 (P=0.003) fold higher in NAG, AG/P and GC cancer patients, respectively, compared to H subjects (FIG. 1A). The analysis of the distribution of samples according to mtDNA values, shows that mtDNA>5 is observed in 89% of AG/P samples, compared to 32% in the healthy group. More precisely, mtDNA>6.3 leads to predict patients with pre-neoplasia (AG/P) with a sensitivity of 66.6% and a specificity of 65% (Table 2). Receiver Operative Characteristics (ROC) curves of mtDNA data reports an Area Under Curve (AUC) value of 0.7089 for AG/P samples (FIG. 3).

### Inflammatory Factors, IL-8, IL-17 and TNFα

GC is an inflammation-driven disease. Even though the variation of the levels of inflammatory mediators as IL-8; IL-17 and TNFα should not be specific of the presence of gastric pre/neoplasia (AG/P), their variation can help to identify patients in which the gastric malignant process is initiated or in progress. In our previous study on the Mexican cohort, high plasmatic level of IL-8 in combination with the measure of mtDNA allowed to improve the detection of GC patients (6). As indicated in the methodology section, plasmatic levels of IL-8, IL-17 and TNFα were evaluated by commercial ELISA assay on all samples of the present AP-HP cohort. As reported in FIG. 1C, mean IL-8 values increased with the stage of the gastric lesions and are 2- (P=0.0098), 1.6- and 3- (P<0.0001) fold higher in NAG, AG/P and GC samples compared to H, respectively. Importantly, 94% of H samples showed IL-840 ng/ml compared to only 39% in GC patients. In addition, IL-8>40ng/ml is observed in 33% and 61% of AG/P and GC samples (FIG. 1D). The calculation of cut-off values according to “not healthy” decision rule indicates that IL-8>17.7pg/ml corresponds to not healthy samples with a sensitivity of 73.2% and a specificity of 72.3% (Table 2). In addition, IL-8>29.4 pg/ml allows to predict patients with cancer (GC), with sensitivity and specificity of 74.6% and 72.9%, respectively (Table 2).

Interestingly IL-17 led to distinguish with no ambiguity between H subjects and patients either with NAG, AG/P or GC lesions. One-hundred percent of NAG, AG/P and GC samples showed IL-17 plasmatic levels>20 pg/ml. In contrast, IL-17≤20 pg/ml is observed in 100% of H samples (FIG. 1G and 1H). More precisely, the calculation of cut-off values according to “not healthy” decision rule indicates that IL-17>41 pg/ml corresponds to not healthy samples with sensitivity and specificity of 100% (Table 2). The good biomarker property of IL-17 is also indicated by ROC curve analysis, with an AUC value of 0.75, 0.72 and 0.67 for NAG, AG/P and GC samples, respectively using the decision rules “if the biomarker quantity is superior (or, depending upon the configuration, inferior, or superior or equal, or inferior or equal) to x, then the patients is H or NAG or AG/P or GC”, where x is a cut off value. (FIG. 3). An AUC of 1 is observed for healthy (H) samples using the decision rule “if the biomarker quantity is inferior (or, depending upon the configuration, superior, or superior or equal, or inferior or equal) to x, then the patients is H or NAG or AG/P or GC” (FIG. 4).

A genetic polymorphism of TNF-α has been previously associated with increased GC risk (13). The measure of plasmatic levels of TNF-α showed higher values of 1.6-; 1.4- and 1.6-fold in NAG, AG/P and GC samples compared to healthy subjects. Similarly to IL-17, TNF-α>80 pg/ml observed in 100%, 87% and 93% of NAG, AG/P and GC samples compared to 26% of H subjects (FIG. 1E and 1F). More precisely, the calculation of cut-off values according to “not healthy” decision rule indicates that TNF-α>74 pg/ml corresponds to not healthy (H) samples with both sensitivity and specificity of 98.8% and 72.3% respectively (Table 2). Accordingly, an AUC values of 0.7954 is obtained using the decision rules “if the biomarker quantity is inferior (or, depending upon the configuration, superior, or superior or equal, or inferior or equal) to x, then the patients is H or NAG or AG/P or GC”, where x is a cut off value. (FIG. 4).

### Upstream Stimulating Factors USF1 and USF2

USF1 and USF2 are pleiotropic transcription factors involved in the regulation of several genes related to important cellular functions as immune response, cell proliferation and maintenance of genome stability (14). These factors have been previously proposed as tumor suppressors (15). A recent study from our team, reported that depletion of USF1 in gastric biopsies from GC patients is associated with a worse prognosis (16). Thus, USF1 could be a potential biomarker candidate to identify patients at risk of GC.

In the present study, USF1 plasmatic levels are 3.6-fold higher in GC patients compared to H subjects (P=0.0002), with 36% and 0% of GC and H samples with USF1>300 pg/ml, respectively (FIGS. 1I and 1J). In that case, the AUC value as determined by ROC curve analysis is 0.774 for GC, according to the decision rule “if the biomarker quantity is superior (or, depending upon the configuration, inferior, or superior or equal, or inferior or equal) to the x cut off value, then the patients is H or NAG or AG/P or GC” (FIG. 3). Data obtained by measuring USF2 did not show significant differences in plasma levels among the samples. However, 89%, 76% and 77% of NAG, AG/P and GC samples respectively, showed USF2>10 pg/ml compared to only 51% for healthy individuals (FIG. 1K and IL).

### Haptoglobin

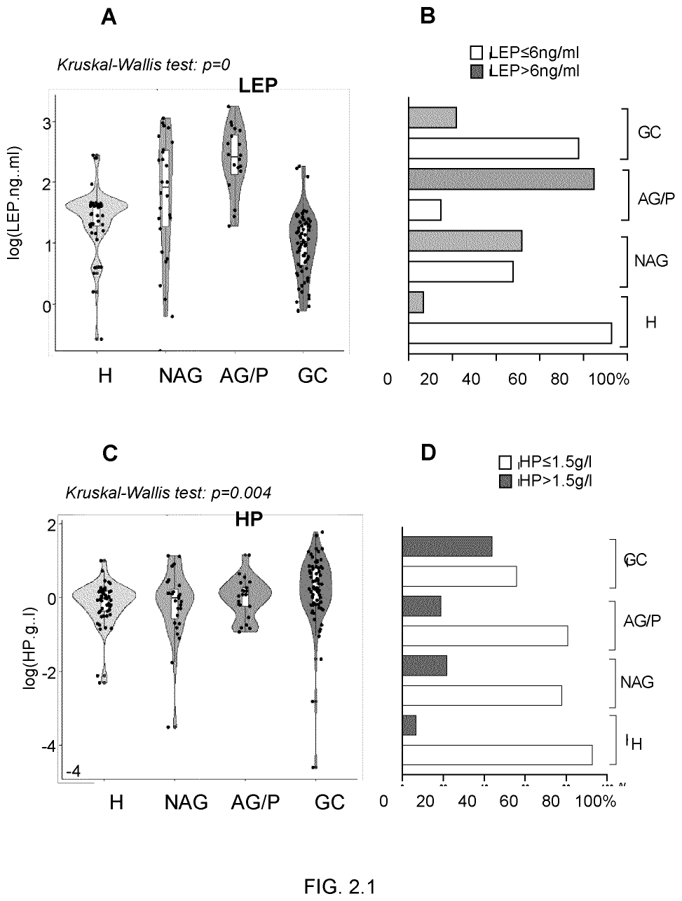

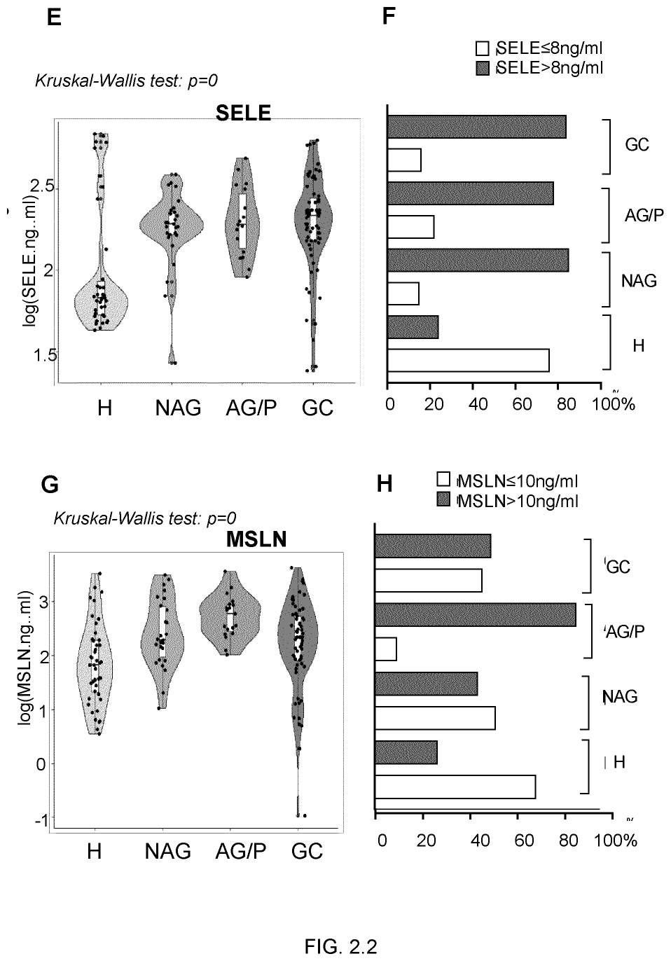

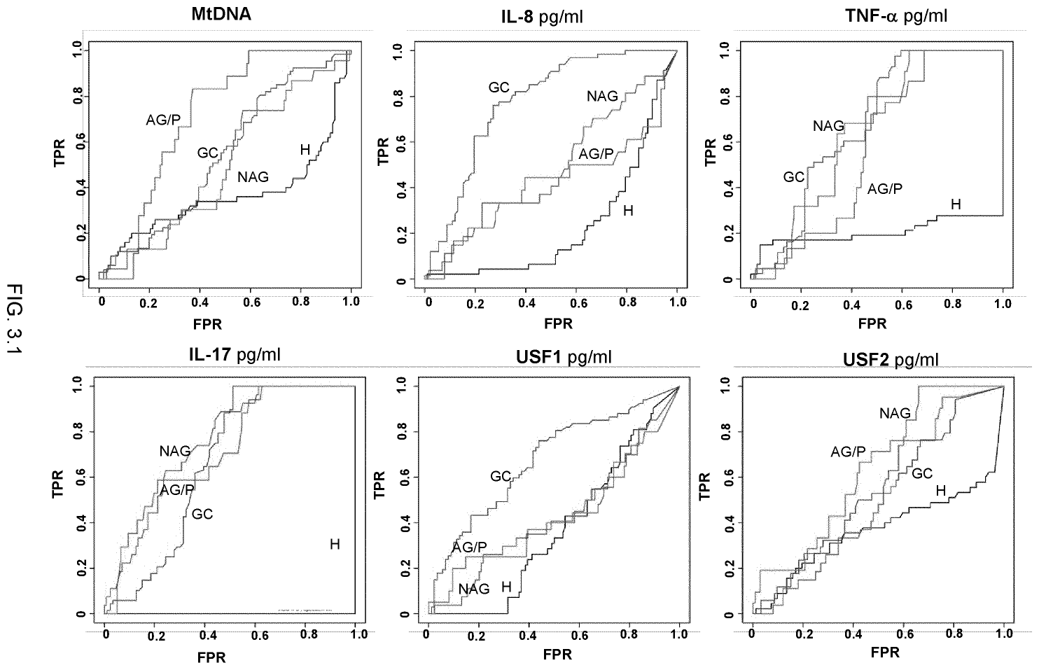

In blood plasma, haptoglobin (HP) binds to free hemoglobin. High serum HP level is associated with tumor progression and poor prognosis as reported in non-small cell lung cancer (17). Serum HP has also been proposed as a novel molecular biomarker to predict colorectal cancer (CRC) hepatic metastasis (18). Recently, aberrant glycosylation of serum HP has been associated with GC (19). In the present study, mean HP plasmatic level is higher in GC samples compared to H (1.7-fold; P=0.0006) (FIG. 2C), with 44% of GC patients with a HP level>1.5 g/l compared to 7% for H subjects (FIG. 2D).

Biomarker Candidates Identified by Proteome Profiler Analysis Biomarker candidates were also searched by proteome profiler analysis, consisting in membranes-based antibody array allowing the parallel determination of the relative levels of selected Human XL oncology pathways proteins (84 cancer-related proteins), as described in the Methodology section. Three candidates including Leptin (LEP), E-Selectin (SELE) and Mesothelin (MSLN) were selected from Oncology pathway arrays, according to the significant variation of their plasmatic level in AG/P and GC samples compared to H. They were then quantified on all samples from the cohort, using specific commercial ELISA assay as indicated in the methodology section.

### Leptin

LEP is a candidate of special interest due to its role as a digestive peptide hormone. LEP is an inducer of inflammatory cytokines. Its deregulation has been reported in a large variety of malignancies including gastrointestinal. In CRC, its expression increases gradually from normal mucosa to adenocarcinoma with high grade dysplasia (20). High leptin serum levels have been associated with an increased risk of gastric intestinal metaplasia and GC (21, 22). In the present study, the mean LEP plasmatic level is 3-fold higher in AG/P compared to H and GC samples (P<0.0001). In addition, 85% of AG/P samples show a LEP level>6ng/ml compared to only 7% in the H group (FIG. 2A and 2B). LEP is a good candidate to identified samples from patients with pre-neoplasia as also demonstrated by the ROC curve that determined an AUC value of 0.705 for AG/P (FIG. 3). The calculation of cut-off indicates that LEP>7.1 ng/ml corresponds to patients with pre-neoplasia (AG/P) with a sensitivity of 75% and a specificity of 86.2% (Table 2). In addition, LEP<4.1 ng/ml leads to predict GC patients with a sensitivity of 82.3% and a specificity of 72.3%. Furthermore, lower values of LEP<2 ng/ml, if not H corresponds to GC samples with a sensitivity of 100% and a specificity of 94.9% (Table 2).

### E-Selectin/CD62E

Selectins are glycoproteins. E-Selectin (SELE) is expressed on endothelial cells under NFκB-mediated transcriptional regulation. Its expression is crucial to control leukocytes accumulation during inflammation. SELE plasmatic levels increase as soon as the NAG stage (P<0.0001 vs healthy) (FIG. 2E), with 85%, 78% and 84% of NAG, AG/P and GC samples with SELE level>8 ng/ml compared to 24% in healthy samples (FIG. 2F). The calculation of cut-off value shows that SELE>7.1 ng/ml corresponds to not healthy samples with a sensitivity of 89.3% and a specificity of 76.3% (Table 2). As indicated in FIG. 3, by ROC curve analysis the best AUC value is 0.7565 for GC samples, using the decision rule, if the biomarker quantity is superior (or, depending upon the configuration, inferior, or superior or equal, or inferior or equal) to x, then the patients is H or NAG or AG/P or GC″, where x is a cut off value.

### Mesothelin

Mesothelin (MSLN) may be involved in cell adhesion. MSLN has been previously reported overexpressed in several human tumors (23). In our study, mean MSLN plasmatic level is 2-fold higher in AG/P samples (P<0.0001 vs H) (FIG. 2G), with 90% of samples with MSLN>10 ng/ml, compared to 28% in the H group. The ROC curve analysis also indicates that MSLN can be considered as a valuable biomarker candidate, with an AUC 0.7433, to identified patients with AG/P (FIG. 3). If MSNL>8.3 ng/ml the sample is not in the Healthy group and MSLN>10.8 ng/ml leads to predict AG/P patients with a sensitivity of 84.2% and a specificity of 63.1% (Table 2).

TABLE 2
List of the main interesting decision rules with corresponding
cut-off values, leading to predict “Not healthy” and patients
either with pre-neoplasia (AG/P) or cancer (GC), for candidates
analyzed by ELISA.

| Main interesting decision rules | | |
|---|---|---|
| “Not healthy” decision rules | True | False |
| In this situation, if marker | Positive | Positive |
| value > x then the patient is not | Rate | Rate |
| healthy (H) | % | % |
| IL-17 > 41 pg/ml | 100 | 0 |
| TNF > 74 pg/ml | 98.8 | 27.7 |
| SELE > 7.1 ng/ml | 89.3 | 23.7 |
| MSLN > 8.3 ng/ml | 76.2 | 38.6 |
| IL-8 > 17.7 pg/ml | 73.2 | 27.7 |
| Decision rules to detect pre-neoplasia: | | |
| In this situation, if marker value > x | | |
| pre-neoplasia (AG/P)then the patient has | | |
| mtDNA > 6.3 | 66.6 | 35 |
| MSLN > 10.8 ng/ml | 84.2 | 36.9 |
| LEP > 7.1 ng/ml | 75 | 13.8 |
| Decision rules to detect gastric cancer | | |
| In this situation if marker value > x or < x | | |
| depending on the analyzed parameter | | |
| (see below) then the patient has | | |
| cancer (GC) | | |
| LEP < 4.1 ng/ml | 82.3 | 27.7 |
| IL-8 > 29.4 pg/ml | 74.6 | 27.1 |
| HP > 1.2 g/l | 60.3 | 29.6 |
| In this situation, if marker value < x | | |
| and not healthy then | | |
| the patient has cancer (GC) | | |
| LEP < 2 ng/ml | 100 | 5.1 |

As reported in FIG. 5, the analysis of the related-function of all these selected biomarker candidates shows connection between SELE, HP, IL-8 (CXCL-8), IL-17 and TNFα altogether related to inflammatory response. As LEP, they have been previously suggested as biomarkers in diagnosis and prognosis evaluation of pancreatitis (24). LEP, also connected with HP, SELE and TNFα is related to signal transduction as USF1 and USF2. Interestingly, MSLN has been already mentioned as protein biomarker for ovarian cancer as LEP, HP, IL-8 (CXCL-8), IL-17 and TNFα (25).

### Biomarker Candidates Identified Through Large-Scale Screening of Plasma Proteins by Mass Spectrometry-Based Proteomics (MS)

In order to get a larger panel of candidate biomarkers and to define a signature profile that will allow to improve as much as possible the early detection of patients at risk of GC, a pilot high-throughput proteomic study has been developed with the UTechS MSBio platform at the Institut Pasteur. This study included the same four groups H, NAG, AG/P and GC with 10 representative samples/group. Plasma samples preparation and MS analysis were performed as described in the methodology section.

After plasma depletion, data analysis for spectral library and DIA acquisition, the comparison between the different groups led to identify a total of 224 differentially abundant proteins. When comparing samples from NAG, AG/P and GC groups to H samples, 114, 88 and 136 proteins showed significant variations, respectively, with some of them commonly found differential among the comparisons. According to correlation matrix and hierarchical clustering analysis (see methodology section), quantified plasma proteomes of cancer (or healthy) patients are generally well correlated between them and less with patients affected by other pathologies (FIG. 6A and 6B). It appears that quantified plasma proteomes of pre-neoplasia (AG/P) and NAG patients are globally well correlated together and less correlated with GC patients and H subjects. Partial Least Square—Discriminant Analysis (PLS-DA) was used to investigate the proteomic differences between the four group samples (H, NAG, AG/P, GC). PLS-DA plot displayed a good separation between H subjects and other pathologies (FIG. 6C). However, it fails to separate clearly NAG, AG/P and GC, indicating that plasma proteome of these three pathologies are closed but different from H individuals.

In order to select a subset of proteins responsible for separating the groups, a sparse PLS-DA was used. This method selects a set of 49 potential biomarkers with no missing values, which make it possible to clearly distinguish healthy individuals and cancer patients but fails to separate patients suffering from pre-neoplasia (AG/P) and patients suffering from gastritis (NAG) (FIG. 6D), as also indicated by the heatmap reported in FIG. 7 that displays the deviations from their mean intensity level of some of these candidates. Among the most relevant candidates, 5 of them allow to predict AG/P patients: IGFBP3, IGFALS, KIF20B, DCD, MAN2A1 and 6 for GC patients: ATAD3B, DCD, S100A12, TFRC, IGHG1, CSTA listed in Table 3.

TABLE 3
List of protein biomarker candidates identified by ELISA or proteomic analysis. Among
the potential candidates, 4 were identified by proteome array (Prot-array) and confirmed
by ELISA, 29 by mass-spectrometry (prot-MS) among which 12 have been confirmed by
ELISA and 4 were previously selected according to their known action in carcinogenesis
and were tested by ELISA. Importantly, LEP has been confirmed by the 3 approaches and
IL-17, SELE, MSLN and HP by 2 approaches. In bold: candidate biomarkers with the
best predictive properties to detect NAG, AG/P and/or GC according to AUC and residual
deviance evaluation. Marked by “x”: approach used to characterize the different biomarker
candidates. In bold: candidate biomarker validated in ELISA (IL-17 and LEP), and found
in the 10 best combinations of 3 biomarkers for proteins identified by mass spectrometry
(Table 6). ^{¥}Plasma proteins related to cancer according to the Human Protein
Atlas (https://www.proteinatlas.org/humanproteome/pathology).

| | Protein | | Cancer | | Prot- | Prot- | AUC | AUC |
|---|---|---|---|---|---|---|---|---|
| | description | Genes | related^{¥} | ELISA | array | MS | AG/P | GC |
| P00738 | Haptoglobin | HP | ¥ | x | | x | 0.629 | 0.567 |
| P08727 | Keratin, type I | KRT19 | ¥ | | | x | 0 | 0 |
| | cytoskeletal 19 | | | | | | | |
| Q96Q89 | Kinesin-like | KIF20B | | | | x | 0.516 | 0.505 |
| | protein KIF20B | | | | | | | |
| Q96T58 | Msx2-interacting | SPEN | ¥ | | | x | | |
| | protein | | | | | | | |
| P05089 | Arginase-1 | ARG1 | | | | x | 0.630 | 0.712 |
| P15924 | Desmoplakin | DSP | | | | x | | |
| P02533 | Keratin, type I | KRT14 | | | | x | | |
| | cytoskeletal 14 | | | | | | | |
| P27918 | Properdin | CFP | | | | x | | |
| P80511 | Protein S100- | S100A12 | | | | x | 0 | 0.488 |
| | A12 | | | | | | | |
| Q5T9A4 | ATPase Family | ATAD3B | | | | x | 0 | 0.583 |
| | AAA Domain- | | | | | | | |
| | Containing Prot | | | | | | | |
| | 3B | | | | | | | |
| P05154 | Plasma serine | SERPINA5 | ¥ | | | x | | |
| | protease | | | | | | | |
| | inhibitor | | | | | | | |
| Q9UI42 | Carboxypeptidase | CPA4 | | | | x | | |
| | A4 | | | | | | | |
| P14923 | Jonction | JUP | ¥ | | | x | 0 | 0.502 |
| | Plakoglobin | | | | | | | |
| P35908 | Keratin, type II | KRT2 | | | | x | | |
| | cytoskeletal 2 | | | | | | | |
| | epidermal | | | | | | | |
| Q15517 | Corneodesmosin | CDSN | | | | x | | |
| P10643 | Complement | C7 | | | | x | | |
| | component C7 | | | | | | | |
| | precursor | | | | | | | |
| P33908 | Mannosyl- | MAN1A1 | | | | x | | |
| | oligosaccharide | | | | | | | |
| | 1,2-alpha- | | | | | | | |
| | mannosidase | | | | | | | |
| Q16706 | Alpha- | MAN2A1 | | | | x | 0 | 0.463 |
| | mannosidase 2 | | | | | | | |
| P35321 | Cornifin A | SPRRA1 | | | | x | | |
| P42357 | Histidine | HAL | | | | x | | |
| | ammonia lyase | | | | | | | |
| P81605 | Dermcidin | DCD | | | | x | 0.581 | 0.597 |
| P00558 | Phosphoglycer | PGK1 | | | | x | | |
| | ate kinase 1 | | | | | | | |
| P35858 | Insulin-like | IGFALS | | | | x | 0 | 0.513 |
| | growth factor- | | | | | | | |
| | binding protein | | | | | | | |
| | complex acid- | | | | | | | |
| | labile subunit | | | | | | | |
| P00918 | Carbonic | CA2 | | | | x | 0 | 0.567 |
| | anhydrase 2 | | | | | | | |
| P48668 | Keratin, type II, | KRT6C | | | | x | | |
| | cytoskeletal 6C | | | | | | | |
| Q5T749 | Keratinocyte | KPRP | | | | x | | |
| | proline rich | | | | | | | |
| | protein | | | | | | | |
| P00488 | Coagulation | F13A1 | ¥ | | | x | | |
| | factor XIII A | | | | | | | |
| | chain | | | | | | | |
| PODJ18/ | Serum amyloid | SAA1/ | | | | x | | |
| PODJ19 | A1 | SAA2 | | | | | | |
| | protein/Serum | | | | | | | |
| | amyloid A2 | | | | | | | |
| | protein | | | | | | | |
| P18428 | LPS-binding | LBP | | | | x | | |
| | protein | | | | | | | |
| P41159 | Leptin | LEP | ¥ | x | | x | 0.685 | 0.61 |
| P10145 | Interleukin-8 | IL-8 | ¥ | x | | | 0 | 0.725 |
| Q16552 | Interleukin-17 | IL-17 | ¥ | x | | | 0 | 0.731 |
| P22415 | Upstream | USF1 | | x | | | 0 | 0.597 |
| | stimulating | | | | | | | |
| | factor 1 | | | | | | | |
| Q15853 | Upstream | USF2 | | x | | | 0.562 | 0 |
| | stimulating | | | | | | | |
| | factor 2 | | | | | | | |
| P16581 | E-Selectin | SELE | ¥ | x | | | 0 | 0.639 |
| Q13421 | Mesothelin | MSLN | ¥ | x | | | 0.515 | 0.543 |
| Q5STB3 | Tumor Necrosis | TNF-α | ¥ | x | | | 0 | 0.505 |
| | Factor α | | | | | | | |
| P00533 | Epidermal | EGFR | x | | | | 0.5 | 0.472 |
| | growth factor | | | | | | | |
| | receptor | | | | | | | |
| P40763 | Signal | STAT3 | x | | | | 0.5 | 0.672 |
| | transducer and | | | | | | | |
| | activator of | | | | | | | |
| | transcription 3 | | | | | | | |

### Prediction and Diagnostic Test Using Combinations of 2 and 3 Potential Biomarkers.

### Candidates Identified by ELISA and Proteome Profiler

The same analysis has been performed for candidates identified by ELISA and proteome profiler array. The deduced ROC curves and AUC specific to each pathology for a combination of two biomarker candidates are reported in FIG. 8A. As above for each pathology, it is possible to evaluate the number of times a protein appears in the models giving the best AUC criteria (FIG. 8B) and the residual deviance of the model (FIG. 9A and 9B). For these candidates, it seems difficult to predict perfectly either gastritis, pre-neoplasia or cancer as no combination of 2 or 3 markers lead to an AUC=1. However, the combination of LEP and mtDNA leads to predict pre-neoplasia (AUC=0.761) and LEP associated with IL-17 (AUC=0.8705) in the case of cancer. Furthermore, as reported in Table 4, LEP associated with HP, USF2, SELE, IL-8 or USF1 allows to predict pre-neoplasia (0.61≤AUC≤0.70) and LEP associated with mtDNA, SELE, IL-8 or USF1 can predict GC (0.74≤AUC≤0.78) as well as IL-17 combined with either mtDNA (AUC=0.76) or TNFα (AUC=0.75). Considering the combination of three biomarker candidates, LEP is always found in the five combinations that predict pre-neoplasia in association with mtDNA in four among the five (0.69≤AUC≤0.73). The AUC values for the eight best combinations of three biomarkers to predict GC are better, comprised between 0.84 and 0.87. For all of them IL-17 and LEP are present. They are associated either with mtDNA, HP, SELE, MSLN, IL-8, USF1 or USF2 (Table 5).

TABLE 4
List of combinations of 2 biomarkers identified by ELISA, with their parameter values and the best corresponding AUC
values to predict NAG, AG/P and GC patients, ranked from the minimum deviance to the highest.
IL.17 pg/ml), HP (g/l), SELE (ng/ml), LEP (ng/ml), MSLN (ng/ml), TNFα (pg/ml), IL.8 (pg/ml), USF1 (pg/ml), USF2 (pg/ml)

| | | Model parameters | | | | | | | | | | | Area Under the Curve | | | |
|---|---|---|---|---|---|---|---|---|---|---|---|---|---|---|---|---|
| v1 | v2 | a0 | b0 | c0 | a1 | b1 | c1 | a2 | b2 | c2 | AIC | Deviance | H | NAG | AG/P | GC |
| IL-17 | LEP | −24.11 | −25.08 | −20.73 | 1.22 | 1.22 | 1.21 | −1.74 | −1.66 | −2.17 | 169.23 | 151.23 | 1.00 | 0.62 | 0.60 | 0.87 |
| TNFα | IL-17 | −9.73 | −9.56 | −8.83 | −0.07 | −0.08 | −0.07 | 0.98 | 0.98 | 0.97 | 172.26 | 154.26 | 1.00 | 0.54 | 0.50 | 0.75 |
| IL-17 | USF2 | −19.70 | −20.12 | −19.39 | 0.57 | 0.57 | 0.57 | 0.00 | 0.01 | 0.01 | 180.58 | 162.58 | 1.00 | 0.53 | 0.52 | 0.71 |
| IL-17 | MSLN | −12.45 | −13.27 | −10.88 | 0.98 | 0.98 | 0.98 | −0.70 | −0.66 | −0.73 | 208.51 | 190.51 | 1.00 | 0.51 | 0.52 | 0.74 |
| mtDNA | IL-17 | −11.11 | −11.60 | −9.95 | −2.40 | −2.32 | −2.35 | 1.75 | 1.75 | 1.75 | 209.22 | 191.22 | 1.00 | 0.54 | 0.50 | 0.76 |
| IL-17 | USF1 | −21.85 | −22.04 | −20.77 | 0.69 | 0.69 | 0.69 | 0.04 | 0.04 | 0.04 | 214.65 | 196.65 | 1.00 | 0.51 | 0.50 | 0.72 |
| IL-8 | IL-17 | −14.38 | −14.39 | −13.53 | −0.07 | −0.08 | −0.06 | 0.64 | 0.63 | 0.63 | 216.49 | 198.49 | 1.00 | 0.53 | 0.50 | 0.73 |
| IL-17 | HP | −16.61 | −16.79 | −16.14 | 0.87 | 0.87 | 0.87 | −5.24 | −5.24 | −4.64 | 216.82 | 198.82 | 1.00 | 0.54 | 0.50 | 0.74 |
| IL-17 | SELE | −18.96 | −19.89 | −18.46 | 1.33 | 1.32 | 1.32 | −2.93 | −2.86 | −2.85 | 222.09 | 204.09 | 1.00 | 0.53 | 0.50 | 0.72 |
| TNFα | LEP | −2.29 | −3.02 | 0.63 | 0.00 | 0.00 | 0.00 | 0.27 | 0.32 | −0.14 | 289.63 | 271.63 | 0.69 | 0.58 | 0.58 | 0.78 |
| SELE | LEP | −3.05 | −4.64 | −1.06 | 0.21 | 0.23 | 0.37 | 0.16 | 0.26 | −0.43 | 298.94 | 280.94 | 0.80 | 0.58 | 0.62 | 0.78 |
| USF2 | LEP | −1.80 | −3.40 | 0.20 | 0.00 | 0.00 | 0.00 | 0.26 | 0.36 | −0.10 | 300.13 | 282.13 | 0.62 | 0.52 | 0.69 | 0.45 |
| TNFα | SELE | −2.25 | −3.19 | −2.69 | 0.00 | 0.00 | 0.00 | 0.20 | 0.27 | 0.31 | 300.96 | 282.96 | 0.77 | 0.50 | 0.50 | 0.62 |
| USF2 | SELE | −2.32 | −3.29 | −4.07 | 0.00 | 0.00 | 0.00 | 0.24 | 0.28 | 0.42 | 303.99 | 285.99 | 0.69 | 0.50 | 0.50 | 0.67 |
| TNFα | MSLN | −1.79 | −2.52 | −0.56 | 0.00 | 0.00 | 0.00 | 0.11 | 0.14 | 0.06 | 310.15 | 292.15 | 0.69 | 0.50 | 0.53 | 0.51 |
| TNFα | USF2 | −0.71 | −1.04 | −0.42 | 0.00 | 0.00 | 0.00 | 0.00 | 0.00 | 0.00 | 312.91 | 294.91 | 0.51 | 0.50 | 0.50 | 0.52 |
| MSLN | LEP | −2.66 | −4.88 | 0.88 | 0.09 | 0.14 | 0.07 | 0.20 | 0.32 | −0.31 | 317.75 | 299.75 | 0.67 | 0.58 | 0.68 | 0.73 |
| mtDNA | LEP | −2.29 | −4.08 | 1.28 | 0.04 | 0.13 | 0.08 | 0.23 | 0.32 | −0.36 | 320.05 | 302.05 | 0.74 | 0.57 | 0.72 | 0.79 |
| TNFα | USF1 | −0.87 | −0.91 | −0.35 | 0.00 | 0.00 | 0.00 | 0.00 | 0.00 | 0.00 | 321.00 | 303.00 | 0.51 | 0.50 | 0.50 | 0.59 |
| HP | LEP | −1.88 | −3.02 | 0.27 | 0.05 | −0.35 | 1.25 | 0.23 | 0.36 | −0.36 | 321.93 | 303.93 | 0.65 | 0.58 | 0.70 | 0.72 |
| USF1 | LEP | −2.26 | −3.69 | 0.81 | 0.00 | 0.00 | 0.00 | 0.24 | 0.34 | −0.26 | 322.01 | 304.01 | 0.63 | 0.57 | 0.61 | 0.74 |
| USF2 | MSLN | −1.43 | −2.55 | −0.94 | 0.00 | 0.00 | 0.00 | 0.10 | 0.13 | 0.07 | 325.76 | 307.76 | 0.63 | 0.53 | 0.54 | 0.56 |
| IL-8 | LEP | −2.67 | −3.87 | 0.48 | 0.02 | 0.02 | 0.02 | 0.27 | 0.37 | −0.23 | 325.93 | 307.93 | 0.74 | 0.55 | 0.64 | 0.77 |
| TNFα | HP | −1.26 | −1.31 | −1.32 | 0.00 | 0.00 | 0.00 | 0.49 | 0.35 | 1.00 | 328.52 | 310.52 | 0.63 | 0.50 | 0.50 | 0.64 |
| mtDNA | USF2 | −0.90 | −1.76 | −0.79 | 0.06 | 0.11 | 0.08 | 0.00 | 0.00 | 0.00 | 328.65 | 310.65 | 0.48 | 0.50 | 0.52 | 0.48 |
| mtDNA | TNFα | −1.08 | −1.53 | −0.45 | 0.02 | 0.08 | 0.05 | 0.00 | 0.00 | 0.00 | 329.95 | 311.95 | 0.50 | 0.50 | 0.50 | 0.48 |
| IL-8 | TNFα | −0.84 | −0.94 | −0.29 | 0.01 | 0.00 | 0.01 | 0.00 | 0.00 | 0.00 | 333.32 | 315.32 | 0.58 | 0.50 | 0.50 | 0.62 |
| USF1 | USF2 | −0.77 | −1.27 | −0.64 | 0.00 | 0.00 | 0.00 | 0.00 | 0.00 | 0.00 | 334.13 | 316.13 | 0.56 | 0.50 | 0.59 | 0.59 |
| IL-8 | SELE | −3.10 | −3.71 | −3.09 | 0.05 | 0.04 | 0.05 | 0.19 | 0.24 | 0.23 | 340.57 | 322.57 | 0.75 | 0.50 | 0.50 | 0.70 |
| IL-8 | USF2 | −0.82 | −1.32 | −0.71 | 0.01 | 0.01 | 0.01 | 0.00 | 0.00 | 0.00 | 341.34 | 323.34 | 0.64 | 0.50 | 0.53 | 0.56 |
| USF2 | HP | −0.80 | −1.29 | −1.46 | 0.00 | 0.00 | 0.00 | 0.35 | 0.31 | 0.95 | 343.22 | 325.22 | 0.60 | 0.50 | 0.54 | 0.67 |
| USF1 | MSLN | −2.10 | −3.42 | −1.24 | 0.00 | 0.00 | 0.00 | 0.12 | 0.17 | 0.10 | 353.56 | 335.56 | 0.69 | 0.50 | 0.52 | 0.61 |
| USF1 | SELE | −2.33 | −3.20 | −2.42 | 0.00 | 0.00 | 0.00 | 0.20 | 0.25 | 0.27 | 353.66 | 335.66 | 0.78 | 0.50 | 0.50 | 0.62 |
| SELE | MSLN | −2.56 | −3.51 | −2.23 | 0.17 | 0.17 | 0.26 | 0.07 | 0.11 | 0.03 | 353.83 | 335.83 | 0.79 | 0.50 | 0.52 | 0.61 |
| SELE | HP | −2.94 | −3.82 | −3.38 | 0.22 | 0.27 | 0.27 | 0.66 | 0.66 | 1.33 | 362.55 | 344.55 | 0.77 | 0.50 | 0.50 | 0.65 |
| mtDNA | MSLN | −2.00 | −3.18 | −0.92 | 0.04 | 0.10 | 0.07 | 0.10 | 0.14 | 0.08 | 363.49 | 345.49 | 0.69 | 0.50 | 0.52 | 0.56 |
| IL-8 | MSLN | −2.06 | −2.83 | −1.14 | 0.02 | 0.01 | 0.02 | 0.10 | 0.14 | 0.07 | 364.82 | 346.82 | 0.72 | 0.50 | 0.53 | 0.64 |
| mtDNA | SELE | −2.44 | −3.51 | −2.13 | −0.02 | 0.04 | 0.01 | 0.24 | 0.28 | 0.29 | 366.12 | 348.12 | 0.81 | 0.50 | 0.50 | 0.64 |
| mtDNA | USF1 | −1.42 | −2.02 | −0.80 | 0.05 | 0.11 | 0.08 | 0.00 | 0.00 | 0.00 | 367.78 | 349.78 | 0.63 | 0.50 | 0.50 | 0.65 |
| MSLN | HP | −2.07 | −2.96 | −1.71 | 0.10 | 0.14 | 0.08 | 0.43 | 0.45 | 1.03 | 370.65 | 352.65 | 0.67 | 0.50 | 0.51 | 0.61 |
| IL-8 | USF1 | −1.26 | −1.38 | −0.85 | 0.0 | 0.01 | 0.02 | 0.00 | 0.00 | 0.00 | 372.91 | 354.91 | 0.71 | 0.50 | 0.50 | 0.73 |
| mtDNA | IL-8 | −1.50 | −1.93 | −0.92 | 0.04 | 0.10 | 0.07 | 0.02 | 0.01 | 0.02 | 382.33 | 364.33 | 0.71 | 0.50 | 0.50 | 0.69 |
| IL-8 | HP | −1.27 | −1.56 | −1.40 | 0.02 | 0.01 | 0.02 | 0.19 | 0.26 | 0.82 | 386.87 | 368.87 | 0.71 | 0.50 | 0.50 | 0.71 |
| USF1 | HP | −1.29 | −1.72 | −1.34 | 0.00 | 0.00 | 0.00 | 0.34 | 0.30 | 0.95 | 388.32 | 370.32 | 0.62 | 0.50 | 0.50 | 0.64 |
| mtDNA | HP | −1.37 | −2.00 | −1.42 | 0.02 | 0.08 | 0.06 | 0.45 | 0.43 | 1.10 | 390.42 | 372.42 | 0.56 | 0.50 | 0.50 | 0.57 |

TABLE 5
List of combinations of 3 biomarkers identified by ELISA, with the best
corresponding AUC values to predict NAG, AG/P and GC patients.

| Combination of 3 biomarkers | NAG | AG/P | GC |
|---|---|---|---|
| IL-17, USF2, SELE | 0.72 | | |
| IL-17, USF2, HP | 0.67 | | |
| LEP, MSLN, HP | | 0.73 | |
| LEP, IL-17, mtDNA | | 0.72 | 0.87 |
| LEP, SELE, mtDNA | | 0.71 | |
| LEP, IL-8, mtDNA | | 0.71 | |
| LEP, MSLN, mtDNA | | 0.69 | |
| IL-17, LEP, HP | | | 0.87 |
| IL-17, LEP, SELE | | | 0.87 |
| IL-17, LEP, MSLN | | | 0.86 |
| IL-17, LEP, IL-8 | | | 0.86 |
| IL-17, LEP, USF1 | | | 0.86 |
| IL-17, LEP, TNFα | | | 0.86 |
| IL-17, LEP, USF2 | | | 0.84 |

### Candidates Identified by Mass Spectrometry Analysis.

Multinomial logistic regression models have been estimated using combinations of 2 or 3 biomarkers among the candidates identified by MS analysis listed in Table 3. Results of the model estimation have been assessed using two approaches. The first one is to predict pathologies from combination of biomarkers using the estimated model and to reduce ROC curves and AUC criteria specific to each pathology (FIG. 10A). Next, for each pathology it is possible to evaluate the number of times a protein appears in the models, giving the best AUC criteria (FIG. 10B). The second approach is to calculate the residual deviances of the models, which correspond to the estimated modeling errors and makes it possible to measure the capacity of the models to predict all pathologies simultaneously (FIG. 11A and 11B).

From our results, it seems difficult to predict perfectly pre-neoplasia and gastritis using only two biomarkers (no combination of biomarkers achieves AUC=1 for these pathologies), while patients suffering from cancer can be perfectly predicted using 30 combinations of two biomarkers (FIG. 10B) including KRT14, CFP, ARG1, SA10012, ATAD3B, KIF20B, SPEN, SERPINA5, DSP, CPA4, KRT19, JUP, KRT2, CDSN, MAN1A1, MAN2A1, SPRR1A, HAL, DCD as reported in Table 6. Importantly, S100A12 is the most often selected candidate to predict GC among the thirty combinations detected with two variables. In addition, gastritis (NAG) patients can be predicted using ten combinations of two biomarkers including PGK1, CFP, IGFALS, KRT19, CPA4, CA2, SERPINA5, MAN2A1 (0.8≤AUC≤0.85) and ten combinations of two biomarkers among which PGK1, CFP, KIF20B, SPEN, JUP, KRT6C, CDSN, KPRP, F13A1, SAA1, LBP, DSP (Table 6), allow to predict patients with pre-neoplasia (AG/P). When using three biomarkers, all pathologies can be perfectly predicted. Quite interestingly, the three combinations of biomarkers giving the lowest residual deviance: KRT19, KIF20B and SPEN; ARG1, KIF20B and SPEN; DSP, KIF20B and SPEN, give a perfect prediction of all the pathologies (AUC=1) for NAG, AG/P and GC. KRT19, KIF20B and SPEN are of special relevance as cancer biomarkers. Keratin 19 (KRT19) serves an important role in different types of cancer and as prognosis marker (26) (27). Kinesin family member 20B (KIF20B) might promote cancer development due to its effect on cell proliferation and apoptosis. Its high expression has been associated with advanced tumor stage and poor prognosis as example in hepatocellular carcinoma (28). In addition, Msx2-interacting protein (SPEN) has been suggested as a novel tumor suppressor and regulates the Notch pathway (29). As indicated in Table 3, KIF20B and SPEN are among cancer-related plasma proteins as JUP, SERPINA5 and F13A1.

TABLE 6
List of combinations of 2 and 3 biomarkers and corresponding
AUC value to predict NAG, AG/P and GC patients.

| Combination of | | | | Combination of | | | |
|---|---|---|---|---|---|---|---|
| 2 biomarkers | NAG | AG/P | GC | 3 biomarkers | NAG | AG/P | GC |
| PGK1, CFP | 0.825 | 0.8 | 0.925 | KRT19, | 1 | 1 | 1 |
| | | | | KIF20B, SPEN | | | |
| PGK1, | 0.775 | 0.75 | 0.875 | ARG1, | 1 | 1 | 1 |
| IGFALS | | | | KIF20B, SPEN | | | |
| KRT19, | 0.85 | 0.8 | 0.825 | DSP, KIF20B, | 1 | 1 | 1 |
| IGFALS | | | | SPEN | | | |
| SPRR1A, | 0.825 | 0.7 | 0.875 | HAL, KIF20B, | 1 | 1 | 1 |
| IGFALS | | | | SPEN | | | |
| IGFALS, | 0.775 | 0.725 | 0.9 | C7, KIF20B, | 1 | 1 | 1 |
| CPA4 | | | | SPEN | | | |
| PGK1, CA2 | 0.8 | 0.675 | 0.85 | JUP, KIF20B, | 1 | 1 | 1 |
| | | | | SPEN | | | |
| SERPINA5, | 0.8 | 0.7 | 0.9 | KIF20B, | 1 | 1 | 1 |
| KRT19 | | | | SPEN, CPA4 | | | |
| SERPINA5, | 0.8 | 0.65 | 0.875 | SPRR1A, | 1 | 1 | 1 |
| MAN2A1 | | | | KIF20B, SPEN | | | |
| SERPINA5, | 0.75 | 0.725 | 1 | MAN2A1, | 1 | 1 | 1 |
| CPA4 | | | | KIF20B, SPEN | | | |
| MAN2A1, | 0.8 | 0.8 | 0.95 | KRT14, | 1 | 1 | 1 |
| KIF20B | | | | KIF20B, SPEN | | | |
| KIF20B, | 0.725 | 0.9 | 0.7 | | | | |
| SPEN | | | | | | | |
| JUP, KRT6C | 0.8 | 0.875 | 0.875 | | | | |
| JUP, CDSN | 0.775 | 0.875 | 0.75 | | | | |
| JUP, KPRP | 0.75 | 0.825 | 0.75 | | | | |
| F13A1, | 0.65 | 0.85 | 0.75 | | | | |
| SAA1(SAA2) | | | | | | | |
| KRT19, LBP | 0.8 | 0.85 | 0.75 | | | | |
| DSP, KPRP | 0.775 | 0.85 | 0.775 | | | | |
| DSP, CDSN | 0.775 | 0.85 | 0.775 | | | | |
| KRT2, CDSN | 0.7 | 0.85 | 0.775 | | | | |
| KRT14, CFP | 0.6 | 0.65 | 1 | | | | |
| ARG1, CFP | 0.7 | 0.7 | 1 | | | | |
| ARG1, | 0.7 | 0.7 | 1 | | | | |
| S100A12 | | | | | | | |
| ARG1, | 0.6 | 0.65 | 1 | | | | |
| ATAD3B | | | | | | | |
| ARG1, | 0.725 | 0.75 | 1 | | | | |
| KIF20B | | | | | | | |
| ARG1, SPEN | 0.7 | 0.7 | 1 | | | | |
| SERPINA5, | 0.725 | 0.725 | 1 | | | | |
| DSP | | | | | | | |
| KRT19, | 0.775 | 0.775 | 1 | | | | |
| S100A12 | | | | | | | |
| KRT19, SPEN | 0.725 | 0.675 | 1 | | | | |
| JUP, KIF20B | 0.675 | 0.725 | 1 | | | | |
| DSP, | 0.625 | 0.625 | 1 | | | | |
| S100A12 | | | | | | | |
| DSP, | 0.65 | 0.675 | 1 | | | | |
| ATAD3B | | | | | | | |
| DSP, KIF20B | 0.725 | 0.75 | 1 | | | | |
| CFP, KRT2 | 0.7 | 0.7 | 1 | | | | |
| CFP, CDSN | 0.775 | 0.775 | 1 | | | | |
| CFP, CPA4 | 0.65 | 0.675 | 1 | | | | |
| MAN1A1, | 0.65 | 0.675 | 1 | | | | |
| SPRR1A | | | | | | | |
| SPRR1A, | 0.7 | 0.7 | 1 | | | | |
| S100A12 | | | | | | | |
| HAL, | 0.75 | 0.725 | 1 | | | | |
| S100A12 | | | | | | | |
| HAL, KIF20B | 0.8 | 0.825 | 1 | | | | |
| HAL, | 0.75 | 0.725 | 1 | | | | |
| ATAD3B | | | | | | | |
| HAL, SPEN | 0.725 | 0.675 | 1 | | | | |
| S100A12, | 0.6 | 0.65 | 1 | | | | |
| DCD | | | | | | | |
| S100A12, | 0.675 | 0.65 | 1 | | | | |
| MAN2A1 | | | | | | | |
| S100A12, | 0.725 | 0.75 | 1 | | | | |
| CPA4 | | | | | | | |
| MAN2A1, | 0.7 | 0.7 | 1 | | | | |
| SPEN | | | | | | | |
| ATAD3B, | 0.65 | 0.675 | 1 | | | | |
| CPA4 | | | | | | | |
| KIF20B, CPA4 | 0.725 | 0.75 | 1 | | | | |
| SPEN, CPA4 | 0.725 | 0.675 | 1 | | | | |

TABLE 7
variation profiles of the biomarkers identified by mass spectrometry-based proteomic,
expressed as log2 ratio, comparing their differential expression between healthy and
NAG, AG/P and GC or between two different stages of the gastric carcinogenesis cascade.
The bottom numbers in italics correspond to P values. The difference between
two groups, for example cancer vs healthy for a given candidate biomarker
will be considered statistically significant if the associated pvalue is >0.05.

| | | AVG Log2 Ratio | | | | | |
|---|---|---|---|---|---|---|---|
| | | Cancer/ | | | Pre- | Pre- | |
| BIO- | Uniprot | Pre- | Cancer/ | Cancer/ | neoplasia/ | neoplasia/ | Gastritis/ |
| MARKERS | ID | neoplasia | Gastritis | Healthy | Gastritis | Healthy | Healthy |
| KIF20B | Q96Q89 | −0.86 | −0.67 | −0.32 | 0.07 | 0.39 | 0.31 |
| SPEN | Q96T58 | −0.59 | −0.58 | −0.07 | −0.10 | 0.44 | 0.54 |
| KRT19 | P08727 | −1.63 | −0.90 | −0.95 | 0.46 | 1.11 | 0.65 |
| ARG1 | P05089 | −1.41 | −0.84 | −1.62 | 0.47 | 0.49 | 0.02 |
| DSP | P15924 | −1.59 | −1.17 | −1.92 | 0.22 | 0.56 | −0.75 |
| HAL | P42357 | −1.27 | −1.14 | −0.69 | −0.12 | 0.68 | 0.32 |
| C7 | P10643 | 0.61 | 0.54 | 0.19 | −0.07 | −0.42 | −0.35 |
| JUP | P14923 | −1.54 | −1.18 | −2.03 | 0.23 | 0.56 | −0.85 |
| CPA4 | Q9UI42 | −1.39 | −1.06 | −0.49 | 0.20 | 0.71 | 0.52 |
| SPRR1A | P35321 | −2.48 | −2.08 | −1.66 | 0.60 | 0.91 | 0.33 |
| CFP | P27918 | −0.26 | −0.33 | −0.35 | −0.08 | −0.09 | −0.01 |
| IGFALS | P35858 | 0.38 | −0.03 | −0.84 | −0.41 | −1.07 | −0.77 |
| PGK1 | P00558 | −1.19 | −1.03 | −0.33 | −0.14 | 0.36 | 0.19 |
| CA2 | P00918 | 0.65 | −0.23 | 2.09 | −0.74 | 1.44 | 2.18 |
| SERPINA5 | P05154 | −0.10 | −0.32 | −0.29 | −0.23 | −0.20 | 0.03 |
| MAN2A1 | Q16706 | 1.06 | 0.79 | −0.24 | −0.27 | −1.29 | −1.03 |
| KRT6C | P48668 | −1.44 | −0.99 | −1.84 | 0.43 | 0.57 | 0.15 |
| CDSN | Q15517 | −1.55 | −1.00 | −0.84 | 0.53 | 0.65 | 0.20 |
| F13A1 | P00488 | −0.61 | −0.30 | −2.52 | 0.04 | −1.90 | −2.25 |
| SAA1 | PODJI8; | 1.79 | 0.76 | 0.29 | −0.10 | −0.84 | −0.47 |
| (SAA2) | PODJI9 | | | | | | |
| LBP | P18428 | 0.66 | 0.88 | 0.13 | 0.04 | −0.60 | −0.70 |
| KPRP | Q5T749 | −1.65 | −1.07 | −2.20 | 0.58 | 0.85 | −1.14 |
| KRT14 | P02533 | −1.27 | −0.90 | −1.93 | 0.35 | −0.67 | 0.03 |
| S100A12 | P80511 | −0.76 | −0.62 | −0.28 | 0.08 | 0.43 | 0.35 |
| ATAD3B | Q5T9A4 | −0.87 | −0.73 | −0.77 | −0.02 | 0.18 | 0.19 |
| KRT2 | P35908 | −1.55 | −1.09 | −1.97 | 0.38 | 0.46 | −0.88 |
| MAN1A1 | P33908 | −0.06 | −0.18 | −0.21 | −0.12 | −0.15 | −0.03 |
| DCD | P81605 | −1.07 | −1.12 | −0.59 | 0.00 | 0.76 | 0.76 |
| HP | P00738 | 1.11 | 0.77 | 2.07 | −0.34 | 0.96 | 1.30 |
| LEP | P41159 | −1.11 | −1.51 | −1.57 | −1.04 | −0.64 | 0.25 |

| | P Value | | | | | |
|---|---|---|---|---|---|---|
| | Cancer/ | | | Pre- | Pre- | |
| BIO- | Pre- | Cancer/ | Cancer/ | neoplasia/ | neoplasia/ | Gastritis/ |
| MARKERS | neoplasia | Gastritis | Healthy | Gastritis | Healthy | Healthy |
| KIF20B | 5.43E−03 | 6.72E−03 | 1.93E−01 | 7.51E−01 | 1.49E−01 | 2.13E−01 |
| SPEN | 3.96E−02 | 6.43E−03 | 8.18E−01 | 6.80E−01 | 1.86E−01 | 8.96E−02 |
| KRT19 | 8.77E−04 | 3.31E−03 | 1.77E−01 | 2.79E−01 | 9.15E−02 | 2.79E−01 |
| ARG1 | 6.84E−04 | 1.02E−02 | 4.28E−02 | 2.29E−01 | 3.54E−01 | 9.76E−01 |
| DSP | 2.09E−03 | 5.33E−03 | 8.53E−02 | 6.33E−01 | 3.69E−01 | 5.76E−01 |
| HAL | 5.65E−04 | 2.83E−03 | 1.93E−01 | 7.29E−01 | 1.67E−01 | 5.54E−01 |
| C7 | 1.66E−02 | 1.61E−02 | 4.96E−01 | 7.15E−01 | 1.11E−01 | 1.37E−01 |
| JUP | 3.22E−03 | 1.19E−02 | 1.04E−01 | 6.38E−01 | 3.90E−01 | 6.21E−01 |
| CPA4 | 3.37E−04 | 5.75E−03 | 3.29E−01 | 5.12E−01 | 1.67E−01 | 2.78E−01 |
| SPRR1A | 1.31E−03 | 5.39E−03 | 6.87E−02 | 1.35E−01 | 1.76E−01 | 6.29E−01 |
| CFP | 2.33E−01 | 9.60E−02 | 1.39E−01 | 7.04E−01 | 7.03E−01 | 9.53E−01 |
| IGFALS | 1.19E−01 | 8.89E−01 | 8.09E−04 | 1.13E−01 | 1.01E−04 | 1.96E−03 |
| PGK1 | 1.83E−03 | 1.57E−03 | 4.66E−01 | 5.64E−01 | 3.53E−01 | 5.48E−01 |
| CA2 | 4.85E−01 | 6.72E−01 | 2.22E−03 | 1.31E−01 | 1.06E−03 | 1.64E−04 |
| SERPINA5 | 7.04E−01 | 1.56E−01 | 2.62E−01 | 3.44E−01 | 4.71E−01 | 9.00E−01 |
| MAN2A1 | 9.95E−03 | 1.68E−02 | 4.61E−01 | 4.52E−01 | 1.65E−03 | 1.76E−03 |
| KRT6C | 1.71E−04 | 4.24E−03 | 6.22E−02 | 2.60E−01 | 3.04E−01 | 7.95E−01 |
| CDSN | 1.13E−03 | 1.37E−02 | 1.35E−01 | 2.36E−01 | 2.51E−01 | 7.11E−01 |
| F13A1 | 3.49E−01 | 3.09E−01 | 5.66E−07 | 9.02E−01 | 1.62E−05 | 1.05E−06 |
| SAA1 | 9.70E−02 | 9.75E−02 | 5.34E−01 | 7.64E−01 | 6.75E−03 | 7.92E−02 |
| (SAA2) | | | | | | |
| LBP | 2.54E−02 | 9.74E−03 | 6.92E−01 | 8.51E−01 | 6.09E−02 | 2.70E−02 |
| KPRP | 2.61E−04 | 2.34E−02 | 1.09E−01 | 1.94E−01 | 2.26E−01 | 8.32E−01 |
| KRT14 | 2.37E−04 | 4.17E−03 | 3.79E−02 | 3.37E−01 | 4.87E−01 | 9.63E−01 |
| S100A12 | 4.64E−03 | 6.83E−03 | 3.69E−01 | 6.97E−01 | 1.66E−01 | 2.43E−01 |
| ATAD3B | 4.31E−03 | 1.82E−03 | 1.18E−02 | 9.54E−01 | 6.06E−01 | 5.30E−01 |
| KRT2 | 4.09E−04 | 5.68E−03 | 5.11E−02 | 3.50E−01 | 4.10E−01 | 8.87E−01 |
| MAN1A1 | 7.66E−01 | 3.49E−01 | 4.02E−01 | 5.53E−01 | 5.63E−01 | 8.98E−01 |
| DCD | 5.95E−05 | 2.05E−04 | 1.39E−01 | 9.91E−01 | 2.54E−02 | 3.76E−02 |
| HP | 1.40E−01 | 2.89E−01 | 1.58E−02 | 4.77E−01 | 1.09E−01 | 2.82E−02 |
| LEP | 9.40E−03 | 1.27E−03 | 1.37E−04 | 2.56E−02 | 5.91E−02 | 3.88E−01 |

### Confirmation of MS Data by ELISA

Further studies have been carried out to confirm the ability of biomarker candidates identified by MS analysis to predict either preneoplasia (AG/P) or GC. The plasma level of at least 11 proteins among those listed herein were quantified by ELISA on all samples of the cohort, as indicated in Table 3 where the protein names are also mentioned.

As reported in FIG. 13, significant differences are observed for both AG/P and GC groups compared to H, for plasmatic level of ARG1, JUP, MAN2A1, LBP and IGFALS, also including LEP previously measured. DCD plasma levels are significantly lower in AG/P compared to H and GC. In addition, ATAD3B, HP, and CA2 levels showed significant difference between GC and H samples (FIG. 13). ROC analysis led to AUC>0.6 to predict preneoplasia (AG/P) for LEP (AUC=0.685), ARG1 (AUC=0.63) and HP (AUC=0.629), and AUC>0.7 to predict GC for ARG1 (AUC=0.712) (Table 3).

Among these candidates, ARG1 is a key element of the urea cycle, that catalyses the conversion of arginine to ornithine and urea, further metabolized in proline and polyamides, driving collagen synthesis and bioenergetic pathways. It is also involved in the modulation of immune response toward cancer and higher level have been reported in tumor microenvironment of GC (30). In our study, ARG1 is able to predict AG/P with a sensitivity (Sens) of 72% and a specificity (Spec) of 54%, while Sens is 49% but Spec 93% in the case of GC.

According to string analysis (FIG. 14), functional connection is indicated with JUP also known as γ-catenin. JUP is involved in cell-cell junction as desmosome and tight-junction. It is implicated in cytoskeleton rearrangement. Its loss is closely correlated with GC malignancy and poor prognostic (31). Here we observed a global increase of JUP plasma levels in both NAG, AG/P and GC samples, but with an AUC of only 0.5 to predict GC with low Sens (27%) and Spec (73%).

Functionally connected to JUP (FIG. 14), HP binds free hemoglobin. Recently, aberrant glycosylation of serum HP has been associated with GC (9). In the present study, HP>1.5 g/l is observed for 44% of GC patients compared to 7% for H subjects. However, the AUC to predict GC is only 0.567 with a Spec of 69% and Sens of 44%.

LBP is functionally connected with LEP (FIG. 14). LBP is a glycoprotein which binds a variety of bacterial LPS, and plays a role in the innate immune response. We observed a significant increase of LBP plasma levels from NAG to GC stages (FIG. 13). LBP shows AUC of 0.5 and 0.562 to predict AG/P and GC respectively. It is to be noticed that in 100% of H samples LBP is 250 ng/ml while in 73% and 79% of AG/P and GC samples respectively, LBP is >250 ng/ml.

DCD can be cleaved in several peptides with different functions. Its best-known role is as antimicrobial host defence protein. A deregulated DCD expression has been reported in various cancer including GC (32). AUC values determined for DCD to predict AG/P and GC are 0.597 and 0.581, respectively, with Sens:88%; Spec 31% for AG/P and Sens:22%; Spec 94% for GC

ATAD3B plays a role in mitochondrial network organization in stem cells and has been shown to be re-expressed in cancer cells (33). ATAD3B plasma levels are higher in GC samples compared to healthy (FIG. 13), with an AUC of 0.583 for GC.

CA2 contributes to pH regulation in the duodenal upper villous epithelium during proton-coupled peptide absorption. An overexpression of CA2 has been reported in gastrointestinal stromal tumors (34). Higher levels of CA2 are observed in plasma from GC patients compared to H subjects (FIG. 13) but with an AUC of only 0.583.

The two others biomarker candidates also quantified are MAN2A1 and IGFALS. MAN2A1 is involved in the biosynthesis of glycans of which different types have been reported in the serum of GC patients (35). MAN2A1 is associated with AUC≤0.5 for both AG/P and GC. The last one, IGFALS is a serum protein that binds the insulin growth factor (IGF). IGFALS has been previously suggested as a marker for malignant progression in liver cancer (36). As MAN2A1, IGFALS is associated with AUC of 0.5 to predict both AG/P and GC.

In addition to these candidates, we also included in our panel factors susceptible to correspond to potent biomarker because of their known role in inflammation and cancer, as mentioned above for IL-8, TNFα, USF1 and USF2. STAT3 and EGFR also listed in Table 3 are two of these further candidates measured by ELISA on all samples of the cohort.

STAT3 is a key transcription factor, related to cellular response to interleukins, LEP and other growth factors (FIG. 14). Importantly, STAT3 has been previously reported up-regulated in GC (37). STAT3-phosphorylation participates to LEP signalling pathway and contributes to LEP resistance, a main risk factor of obesity (38). Here we observed that STAT3 plasmatic levels are higher in GC patients (FIG. 13). All and 89% of H and NAG samples respectively, show a STAT3 level 250 ng/ml while in most of GC samples (59%) STAT3 is >5 ng/ml with an AUC value of 0.672. (Sens 62.8%; Spec 71.6%).

The second one is EGFR which plays a crucial role in cell proliferation and tumor development. It is overexpressed in 27 to 64% of gastric tumors, and proposed as an indicator of worse outcome in GC patients (39). Higher EGFR levels are observed in GC samples compared to AG/P (FIG. 13), with AUC of only 0.5 and 0.472 for AG/P and GC respectively.

### Prediction and Diagnostic Test Using Combinations of 2 to 6 Biomarker Candidates

The present data as well as the literature on biomarkers discovery argue for the importance of a combination of biomarker panels rather than a single candidate to improve the early detection of cancer patients. Indeed from our results, it seems difficult to predict perfectly pre-neoplasia and/or cancer using a single biomarker as the best AUC values for a single candidate are 0.731 and 0.725 for IL-17 (Sens 50.5%, Spec 95.6%) and IL-8 (Sens 63%, Spec 82%), respectively, to predict GC. In the case of preneoplasia, LEP gives the best AUC value: 0.685 (Sens 94.2%, Spec 42.8%). To improve the detection of the presence of preneoplasia or cancer lesions, multinomial logistic regression models have been estimated using combinations of up to 6 biomarkers among the candidates of which plasma level has been measured by ELISA on all samples of the cohort.

Based on these data, results of the model estimation have been assessed using two approaches. The first one is to predict pathologies from combination of biomarkers using the estimated model and to deduce ROC curves and AUC values specific to each pathology. Next, for each pathology it is possible to evaluate the number of times a protein appears in the models giving the best AUC criteria. The second approach is to calculate the residual deviances of the models, which correspond to the estimated modeling errors and makes it possible to measure the capacity of the models to predict all pathologies simultaneously.

Using these models, we were able to identify the two best signatures according to AUC, Sens and Spec values to predict the presence of gastric preneoplasia (SIG-AGP) and cancer lesions (SIG-GC), including up to 6 biomarkers.

The so-called “SIG-AGP” signature corresponds to the combination of MSLN, HP, LEP, KRT19, IGFALS, EGFR with an AUC value of 0.852, Sens 91.4% with Spec 79% to predict preneoplasia (FIG. 15A).

The so-called “SIG-GC” signature includes IL-17, ARG1, LEP, MSLN, TNFα, SELE with an AUC value of 0.928, Sens 92.9% and Spec 92.7% to predict GC (FIG. 15B).

LEP and MSLN are found in both. Median, Min and Max plasma levels for each biomarker are reported in Table 8.

Importantly, the predictive properties of these two signatures increase with the number of biomarkers. For both, AUC increases from 1 to 6 proteins as well as Spec for SIG-AG/P and Sens for SIG-GC. Concerning SIG-AGP, a Sens of 94% is already observed with LEP as a single biomarker but with a Spec of only 42.8%. Similarly, in the case of SIG-GC, the highest Spec (95.6%) is obtained with a single biomarker, IL-17.

For completeness, Table 9 reports the list of the best combinations of 6 biomarkers to predict AG/P. These combinations correspond to an AUC≥0.8, with Sensitivity between 90 to 96% and Specificity between 71% and 79%.

In addition, as reported in Table 10, a combination of 5 biomarkers: MSNL, LEP, KRT19, MAN2A1, IGFALS can predict preneoplasia, with a higher Sens than SIG-AGP (94.1%) and a Spec of 73.7%.

In addition, to predict GC, an excellent score is also observed with the combination: TNFα, IL-17, MSLN, LEP, KIF20B, ARG1 (AUC: 0.928; Sens 93%, Spec 92.7%), where compared to SIG-GC, SELE is replaced by KIF20B. It is to be noticed that the best Spec (95.6%) for GC is already obtained with just a single biomarker, IL-17 but with lower AUC and Sens (0.731 and 50.6%).

For completeness, Table 11 reports the list of the best combinations of 6 biomarkers to predict GC. These combinations correspond to an AUC≥0.9, with Sensitivity between 87 to 94% and Specificity between 88% and 94%.

TABLE 8
Plasmatic levels of each biomarker identified in the signatures to predict preneoplasia and GC.
Values are measured by ELISA. H: Healthy; NAG: non-atrophic gastritis;
AG/P: atrophic gastritis and preneoplasia; GC: gastric cancer.

| | | | VALUES | | | |
|---|---|---|---|---|---|---|
| | SIGNATURE | | MEDIAN (MIN-MAX) | | | |
| PROTEINS | AG/P | GC | H | NAG | AG/P | GC |
| ARG-1 (NG/ML) | | X | 9.28 (0.8-77) | 5.77 (0-47.2) | 11.65 (0-64) | 19.45 (2.6-177) |
| IL-17 (NG/ML) | | X | 1.93 (1.2-20) | 85.9 (48-692) | 90.7 (50-148) | 64 (41.2-560) |
| EGFR (NG/ML) | X | | 6.06 (0-32) | 5.08 (0-42) | 8.35 (0-28) | 11.54 (0-258) |
| HP (G/L) | X | | 0.96 (0.1-2.7) | 0.98 (0.03-3.1) | 1.08 (0.43.2) | 1.29 (0.01-5.89) |
| IGFALS (NG/ML) | X | | 24.76 (3.16-151) | 14.77 (0.6-140.5) | 14.25 (0-70.5) | 10.66 (0-170.3) |
| KRT19 (NG/ML) | X | | 0.36 (0.04-17) | 0.59 (0-39) | 0.45 (0-61) | 0.87 (0-50.3) |
| LEP (NG/ML) | X | X | 4.95 (0.6-11.54) | 6.19 (0-21) | 11.15 (3.6-26) | 2.58 (0-19.15) |
| MSLN (NG/ML) | X | X | 6.45 (1.7-33.5) | 9.84 (2.8-32.6) | 15.82 (7.5-35) | 10.29 (0.4-40) |
| SELE (NG/ML) | | X | 6.22 (5.12-17) | 9.8 (4.2-13.3) | 9.79 (7-14.7) | 10.35 (4-16.4) |
| TNFa (PG/ML) | | X | 50.65 (42.1-1126) | 125.3 (80-1283) | 109.5 (75-262) | 130.3 (74-1843) |

TABLE 9
List of the best combinations of 6 biomarkers to predict AG/P. These combinations correspond
to an AUC > 0.8, with Sensitivity between 90 to 96% and Specificity between 71% and 79%.

| Biomarker 1 | Biomarker 2 | Biomarker 3 | | Biomarker 4 |
|---|---|---|---|---|
| MESOTHELIN..ng.ml. | HAPTOGLOBINE..g.l. | LEPTINE..ng.ml. | | KRT19..ng.ml. |
| E..SELECTIN..ng.ml. | MESOTHELIN..ng.ml. | LEPTINE..ng.ml. | | KRT19..ng.ml. |
| MESOTHELIN..ng.ml. | LEPTINE..ng.ml. | KRT19..ng.ml. | | IGFALS..ng.ml. |
| MESOTHELIN..ng.ml. | HAPTOGLOBINE..g.l. | LEPTINE..ng.ml. | | KRT19..ng.ml. |
| MESOTHELIN..ng.ml. | LEPTINE..ng.ml. | S100A12..pg.ml. | | KRT19..ng.ml. |
| MESOTHELIN..ng.ml. | LEPTINE..ng.ml. | KIF20B | | KRT19..ng.ml. |
| MESOTHELIN..ng.ml. | LEPTINE..ng.ml. | KRT19..ng.ml. | | MAN2A1.ng.ml |
| MESOTHELIN..ng.ml. | HAPTOGLOBINE..g.l. | LEPTINE..ng.ml. | | KRT19..ng.ml. |
| MESOTHELIN..ng.ml. | LEPTINE..ng.ml. | KRT19..ng.ml. | | MAN2A1.ng.ml |
| USF1..pg.ml. | MESOTHELIN..ng.ml. | LEPTINE..ng.ml. | | KRT19..ng.ml. |
| MESOTHELIN..ng.ml. | LEPTINE..ng.ml. | KRT19..ng.ml. | | IGFALS..ng.ml. |
| IL.17..pg.ml. | MESOTHELIN..ng.ml. | LEPTINE..ng.ml. | | KRT19..ng.ml. |
| MESOTHELIN..ng.ml. | HAPTOGLOBINE..g.l. | LEPTINE..ng.ml. | | KRT19..ng.ml. |
| IL.17..pg.ml. | MESOTHELIN..ng.ml. | HAPTOGLOBINE..g.l. | | LEPTINE..ng.ml. |
| IL.17..pg.ml. | MESOTHELIN..ng.ml. | LEPTINE..ng.ml. | | KRT19..ng.ml. |
| E..SELECTIN..ng.ml. | MESOTHELIN..ng.ml. | LEPTINE..ng.ml. | | S100A12..pg.ml. |
| IL.17..pg.ml. | MESOTHELIN..ng.ml. | HAPTOGLOBINE..g.l. | | LEPTINE..ng.ml. |
| IL.17..pg.ml. | E..SELECTIN..ng.ml. | MESOTHELIN..ng.ml. | | LEPTINE..ng.ml. |
| USF1..pg.ml. | MESOTHELIN..ng.ml. | HAPTOGLOBINE..g.l. | | LEPTINE..ng.ml. |
| MESOTHELIN..ng.ml. | HAPTOGLOBINE..g.l. | LEPTINE..ng.ml. | | KIF20B |
| IL.17..pg.ml. | MESOTHELIN..ng.ml. | LEPTINE..ng.ml. | | S100A12..pg.ml. |
| IL.8..pg.ml. | IL.17..pg.ml. | MESOTHELIN..ng.ml. | | LEPTINE..ng.ml. |
| IL.17..pg.ml. | MESOTHELIN..ng.ml. | LEPTINE..ng.ml. | | KIF20B |
| IL.17..pg.ml. | MESOTHELIN..ng.ml. | LEPTINE..ng.ml. | | KRT19..ng.ml. |
| IL.17..pg.ml. | MESOTHELIN..ng.ml. | LEPTINE..ng.ml. | | KRT19..ng.ml. |
| MESOTHELIN..ng.ml. | HAPTOGLOBINE..g.l. | LEPTINE..ng.ml. | | S100A12..pg.ml. |
| MESOTHELIN..ng.ml. | HAPTOGLOBINE..g.l. | LEPTINE..ng.ml. | | KRT19..ng.ml. |
| IL.8..pg.ml. | IL.17..pg.ml. | MESOTHELIN..ng.ml. | | LEPTINE..ng.ml. |
| USF1..pg.ml. | E..SELECTIN..ng.ml. | MESOTHELIN..ng.ml. | | LEPTINE..ng.ml. |
| IL.17..pg.ml. | USF1..pg.ml. | MESOTHELIN..ng.ml. | | LEPTINE..ng.ml. |
| IL.17..pg.ml. | USF2.pg.ml. | MESOTHELIN..ng.ml. | | LEPTINE..ng.ml. |
| IL.17..pg.ml. | MESOTHELIN..ng.ml. | HAPTOGLOBINE..g.l. | | LEPTINE..ng.ml. |
| IL.17..pg.ml. | MESOTHELIN..ng.ml. | LEPTINE..ng.ml. | | KRT19..ng.ml. |
| IL.17..pg.ml. | MESOTHELIN..ng.ml. | LEPTINE..ng.ml. | | KRT19..ng.ml. |
| IL.8..pg.ml. | USF2.pg.ml. | MESOTHELIN..ng.ml. | | LEPTINE..ng.ml. |
| IL.17..pg.ml. | USF1..pg.ml. | MESOTHELIN..ng.ml. | | LEPTINE..ng.ml. |
| | | AUC | Sensibility | Specificity |
| Biomarker 5 | Biomarker 6 | Preneoplasia | Preneoplasia | Preneoplasia |
| IGFALS..ng.ml. | EFGR..ng.ml. | 0.85 | 91% | 79% |
| IGFALS..ng.ml. | EFGR..ng.ml. | 0.85 | 92% | 78% |
| CA2..ng.ml. | EFGR..ng.ml. | 0.85 | 96% | 74% |
| MAN2A1.ng.ml | IGFALS..ng.ml. | 0.84 | 94% | 74% |
| MAN2A1.ng.ml | IGFALS..ng.ml. | 0.84 | 94% | 74% |
| MAN2A1.ng.ml | IGFALS..ng.ml. | 0.84 | 94% | 74% |
| IGFALS..ng.ml. | EFGR..ng.ml. | 0.84 | 94% | 74% |
| CA2..ng.ml. | EFGR..ng.ml. | 0.84 | 94% | 74% |
| IGFALS..ng.ml. | LBP..ng.ml. | 0.84 | 94% | 74% |
| MAN2A1.ng.ml | IGFALS..ng.ml. | 0.83 | 95% | 72% |
| JUP..ng.ml. | EFGR..ng.ml. | 0.83 | 93% | 74% |
| MAN2A1.ng.ml | EFGR..ng.ml. | 0.83 | 94% | 72% |
| MAN2A1.ng.ml | LBP..ng.ml. | 0.83 | 92% | 74% |
| KRT19..ng.ml. | MAN2A1.ng.ml | 0.83 | 93% | 72% |
| MAN2A1.ng.ml | IGFALS..ng.ml. | 0.83 | 93% | 72% |
| KRT19..ng.ml. | MAN2A1.ng.ml | 0.83 | 93% | 72% |
| KRT19..ng.ml. | LBP..ng.ml. | 0.83 | 93% | 72% |
| KRT19..ng.ml. | MAN2A1.ng.ml | 0.83 | 93% | 72% |
| KRT19..ng.ml. | EFGR..ng.ml. | 0.83 | 93% | 72% |
| KRT19..ng.ml. | EFGR..ng.ml. | 0.82 | 91% | 74% |
| KRT19..ng.ml. | MAN2A1.ng.ml | 0.82 | 93% | 72% |
| KRT19..ng.ml. | MAN2A1.ng.ml | 0.82 | 93% | 72% |
| KRT19..ng.ml. | MAN2A1.ng.ml | 0.82 | 93% | 72% |
| IGFALS..ng.ml. | EFGR..ng.ml. | 0.82 | 92% | 72% |
| JUP..ng.ml. | EFGR..ng.ml. | 0.82 | 92% | 72% |
| KRT19..ng.ml. | EFGR..ng.ml. | 0.82 | 91% | 74% |
| DCD..ug.ml. | EFGR..ng.ml. | 0.82 | 90% | 74% |
| KRT19..ng.ml. | IGFALS..ng.ml. | 0.82 | 91% | 72% |
| KRT19..ng.ml. | MAN2A1.ng.ml | 0.82 | 93% | 71% |
| KRT19..ng.ml. | MAN2A1.ng.ml | 0.81 | 92% | 71% |
| KRT19..ng.ml. | MAN2A1.ng.ml | 0.81 | 91% | 72% |
| KRT19..ng.ml. | EFGR..ng.ml. | 0.81 | 90% | 72% |
| MAN2A1.ng.ml | ATAD3B..ng.ml. | 0.81 | 92% | 71% |
| IGFALS..ng.ml. | ATAD3B..ng.ml. | 0.81 | 92% | 71% |
| KIF20B | KRT19..ng.ml. | 0.81 | 90% | 72% |
| KRT19..ng.ml. | IGFALS..ng.ml. | 0.81 | 91% | 71% |

TABLE 10
Combinations of 2 to 6 biomarkers selected according to the best AUC value to predict preneoplasia and
cancer lesions. For AG/P, as soon as 4 biomarkers, AUC and Sens are >0.82 and 92%, respectively for
a Spec slightly lower compared to 6 biomarkers 73.7% instead of 77.8%. In the case of GC, a number
of 6 biomarkers allows to improve the sensitivity while the spec is of 95% even with only one protein.

| Nb | | | Sens | Spec | | | Sens | Spec |
|---|---|---|---|---|---|---|---|---|
| biomarkers | Preneoplasia AG/P | AUC | % | % | Cancer GC | AUC | % | % |
| 6 | SELE, MSLN, LEP, | 0.848 | 91.8 | 77.8 | TNF&, IL-17, MSLN, | 0.928 | 93 | 92.7 |
| | KRT19, IGFALS, EGFR | | | | LEP, KIF20B, ARG1 | | | |
| 5 | MSNL, LEP, KRT19, | 0.839 | 94.1 | 73.7 | IL-17, ARG1, LEP, | 0.910 | 88 | 93.9 |
| | MAN2A1, IGFALS | | | | DCD, CA2 | | | |
| 4 | MSNL, LEP, KRT19, | 0.829 | 92.1 | 73.7 | IL-17, ARG1, LEP, | 0.898 | 86.4 | 93.2 |
| | LBP | | | | MSLN | | | |
| 3 | LEP, CA2, LBP | 0.778 | 93.7 | 61.9 | IL-17, ARG1, LEP | 0.880 | 81.9 | 94 |
| 2 | LEP, KRT19 | 0.755 | 94 | 57.1 | IL-17, ARG1 | 0.833 | 72.6 | 94.1 |
| 1 | LEP | 0.685 | 94.2 | 42.8 | IL-17 | 0.731 | 50.6 | 95.6 |

TABLE 11
List of the best combinations of 6 biomarkers to predict GC. These combinations correspond
to an AUC > 0.9, with Sensitivity between 87 to 94% and Specificity between 88% and 94%.

| Biomarker 1 | Biomarker 2 | Biomarker 3 | Biomarker 4 | |
|---|---|---|---|---|
| IL.17..pg.ml. | USF2.pg.ml. | MESOTHELIN..ng.ml. | LEPTINE..ng.ml. | |
| IL.17..pg.ml. | USF2.pg.ml. | MESOTHELIN..ng.ml. | LEPTINE..ng.ml. | |
| IL.17..pg.ml. | USF2.pg.ml. | LEPTINE..ng.ml. | S100A12..pg.ml. | |
| IL.17..pg.ml. | USF2.pg.ml. | MESOTHELIN..ng.ml. | LEPTINE..ng.ml. | |
| IL.17..pg.ml. | USF2.pg.ml. | MESOTHELIN..ng.ml. | LEPTINE..ng.ml. | |
| TNFa..pg.ml. | IL.17..pg.ml. | MESOTHELIN..ng.ml. | LEPTINE..ng.ml. | |
| TNFa..pg.ml. | IL.17..pg.ml. | MESOTHELIN..ng.ml. | HAPTOGLOBINE..g.l. | |
| TNFa..pg.ml. | IL.17..pg.ml. | MESOTHELIN..ng.ml. | LEPTINE..ng.ml. | |
| IL.8..pg.ml. | TNFa..pg.ml. | IL.17..pg.ml. | MESOTHELIN..ng.ml. | |
| TNFa..pg.ml. | IL.17..pg.ml. | USF2.pg.ml. | MESOTHELIN..ng.ml. | |
| TNFa..pg.ml. | IL.17..pg.ml. | MESOTHELIN..ng.ml. | LEPTINE..ng.ml. | |
| TNFa..pg.ml. | IL.17..pg.ml. | USF1..pg.ml. | MESOTHELIN..ng.ml. | |
| IL.17..pg.ml. | USF2.pg.ml. | MESOTHELIN..ng.ml. | LEPTINE..ng.ml. | |
| IL.8..pg.ml. | IL.17..pg.ml. | USF2.pg.ml. | MESOTHELIN..ng.ml. | |
| TNFa..pg.ml. | IL.17..pg.ml. | MESOTHELIN..ng.ml. | LEPTINE..ng.ml. | |
| TNFa..pg.ml. | IL.17..pg.ml. | MESOTHELIN..ng.ml. | LEPTINE..ng.ml. | |
| TNFa..pg.ml. | IL.17..pg.ml. | MESOTHELIN..ng.ml. | LEPTINE..ng.ml. | |
| TNFa..pg.ml. | IL.17..pg.ml. | MESOTHELIN..ng.ml. | S100A12..pg.ml. | |
| IL.17..pg.ml. | LEPTINE..ng.ml. | KRT19..ng.ml. | ARG1..ng.ml. | |
| IL.17..pg.ml. | LEPTINE..ng.ml. | ARG1..ng.ml. | IGFALS..ng.ml. | |
| IL.17..pg.ml. | USF2.pg.ml. | LEPTINE..ng.ml. | ARG1..ng.ml. | |
| IL.17..pg.ml. | USF2.pg.ml. | MESOTHELIN..ng.ml. | LEPTINE..ng.ml. | |
| TNFa..pg.ml. | IL.17..pg.ml. | MESOTHELIN..ng.ml. | LEPTINE..ng.ml. | |
| IL.8..pg.ml. | TNFa..pg.ml. | IL.17..pg.ml. | MESOTHELIN..ng.ml. | |
| IL.17..pg.ml. | HAPTOGLOBINE..g.l. | LEPTINE..ng.ml. | ARG1..ng.ml. | |
| IL.8..pg.ml. | IL.17..pg.ml. | LEPTINE..ng.ml. | ARG1..ng.ml. | |
| IL.17.pg.ml. | LEPTINE..ng.ml. | ARG1..ng.ml. | IGFALS..ng.ml. | |
| TNFa..pg.ml. | IL.17..pg.ml. | MESOTHELIN..ng.ml. | LEPTINE..ng.ml. | |
| IL.17..pg.ml. | USF2.pg.ml. | MESOTHELIN..ng.ml. | LEPTINE..ng.ml. | |
| IL.17..pg.ml. | MESOTHELIN..ng.ml. | LEPTINE..ng.ml. | KRT19..ng.ml. | |
| IL.17..pg.ml. | MESOTHELIN..ng.ml. | LEPTINE..ng.ml. | ARG1..ng.ml. | |
| IL.17..pg.ml. | LEPTINE..ng.ml. | ARG1..ng.ml. | DCD..ug.ml. | |
| TNFa..pg.ml. | MESOTHELIN..ng.ml. | LEPTINE..ng.ml. | ARG1..ng.ml. | |
| IL.17..pg.ml. | IL.17..pg.ml. | MESOTHELIN..ng.ml. | HAPTOGLOBINE..g.l. | |
| IL.17..pg.ml. | LEPTINE..ng.ml. | ARG1..ng.ml. | MAN2A1.ng.ml | |
| IL.8..pg.ml. | HAPTOGLOBINE..g.l. | LEPTINE..ng.ml. | ARG1..ng.ml. | |
| IL.8..pg.ml. | IL.17..pg.ml. | LEPTINE..ng.ml. | KRT19..ng.ml. | |
| IL.17..pg.ml. | IL.17..pg.ml. | LEPTINE..ng.ml. | ARG1..ng.ml. | |
| IL.17..pg.ml. | HAPTOGLOBINE..g.l. | LEPTINE..ng.ml. | ARG1..ng.ml. | |
| IL.17..pg.ml. | LEPTINE..ng.ml. | ARG1..ng.ml. | MAN2A1.ng.ml | |
| IL.17..pg.ml. | IL.17..pg.ml. | MESOTHELIN..ng.ml. | ARG1..ng.ml. | |
| IL.17..pg.ml. | USF1..pg.ml. | USF2.pg.ml. | MESOTHELIN..ng.ml. | |
| IL.17..pg.ml. | LEPTINE..ng.ml. | KRT19..ng.ml. | ARG1..ng.ml. | |
| IL.17..pg.ml. | LEPTINE..ng.ml. | S100A12..pg.ml. | KRT19..ng.ml. | |
| TNFa..pg.ml. | TNFa..pg.ml. | IL.17..pg.ml. | LEPTINE..ng.ml. | |
| IL.17..pg.ml. | IL.17..pg.ml. | MESOTHELIN..ng.ml. | LEPTINE..ng.ml. | |
| IL.17..pg.ml. | LEPTINE..ng.ml. | ARG1..ng.ml. | IGFALS..ng.ml. | |
| IL.17..pg.ml. | LEPTINE..ng.ml. | KIF20B | ARG1..ng.ml. | |
| IL.17..pg.ml. | LEPTINE..ng.ml. | ARG1..ng.ml. | MAN2A1.ng.ml | |
| IL.17..pg.ml. | LEPTINE..ng.ml. | S100A12..pg.ml. | ARG1..ng.ml. | |
| IL.17..pg.ml. | MESOTHELIN..ng.ml. | LEPTINE..ng.ml. | KIF20B | |
| IL.17..pg.ml. | LEPTINE..ng.ml. | ARG1..ng.ml. | DCD..ug.ml. | |
| IL.17..pg.ml. | LEPTINE..ng.ml. | S100A12..pg.ml. | ARG1..ng.ml. | |
| IL.17..pg.ml. | MESOTHELIN..ng.ml. | LEPTINE..ng.ml. | ARG1..ng.ml. | |
| IL.17..pg.ml. | MESOTHELIN..ng.ml. | LEPTINE..ng.ml. | S100A12..pg.ml. | |
| IL.17..pg.ml. | LEPTINE..ng.ml. | KRT19..ng.ml. | ARG1..ng.ml. | |
| IL.17..pg.ml. | MESOTHELIN..ng.ml. | LEPTINE..ng.ml. | KRT19..ng.ml. | |
| IL.17..pg.ml. | MESOTHELIN..ng.ml. | LEPTINE..ng.ml. | ARG1..ng.ml. | |
| IL.17..pg.ml. | LEPTINE..ng.ml. | ARG1..ng.ml. | DCD..ug.ml. | |
| IL.17..pg.ml. | USF1..pg.ml. | LEPTINE..ng.ml. | ARG1..ng.ml. | |
| IL.17..pg.ml. | IL.17..pg.ml. | MESOTHELIN..ng.ml. | LEPTINE..ng.ml. | |
| IL.17..pg.ml. | MESOTHELIN..ng.ml. | LEPTINE..ng.ml. | ARG1..ng.ml. | |
| IL.8..pg.ml. | LEPTINE..ng.ml. | KIF20B | ARG1..ng.ml. | |
| IL.17..pg.ml. | IL.17..pg.ml. | LEPTINE..ng.ml. | ARG1..ng.ml. | |
| IL.8..pg.ml. | LEPTINE..ng.ml. | S100A12..pg.ml. | ARG1..ng.ml. | |
| IL.17..pg.ml. | IL.17..pg.ml. | LEPTINE..ng.ml. | ARG1..ng.ml. | |
| | | AUC | Sensibility | Specificity |
| Biomarker 5 | Biomarker 6 | Cancer | Cancer | Cancer |
| KRT19..ng.ml. | ARG1..ng.ml. | 0.91 | 94% | 88% |
| ARG1..ng.ml. | MAN2A1.ng.ml | 0.92 | 94% | 91% |
| ARG1..ng.ml. | ATAD3B..ng.ml. | 0.92 | 93% | 91% |
| ARG1..ng.ml. | DCD..ug.ml. | 0.91 | 93% | 88% |
| ARG1..ng.ml. | EGFR..ng.ml. | 0.91 | 93% | 88% |
| KIF20B | ARG1..ng.ml. | 0.93 | 93% | 93% |
| LEPTINE..ng.ml. | ARG1..ng.ml. | 0.92 | 93% | 90% |
| S100A12..pg.ml. | ARG1..ng.ml. | 0.92 | 93% | 90% |
| S100A12..pg.ml. | ARG1..ng.ml. | 0.91 | 93% | 88% |
| LEPTINE..ng.ml. | ARG1..ng.ml. | 0.92 | 93% | 91% |
| ARG1..ng.ml. | MAN2A1.ng.ml | 0.91 | 93% | 90% |
| LEPTINE..ng.ml. | ARG1..ng.ml. | 0.91 | 93% | 90% |
| S100A12..pg.ml. | ARG1..ng.ml. | 0.92 | 93% | 91% |
| LEPTINE..ng.ml. | ARG1..ng.ml. | 0.90 | 93% | 88% |
| ARG1..ng.ml. | STAT3..ng.ml. | 0.91 | 92% | 90% |
| ARG1..ng.ml. | IGFALS..ng.ml. | 0.91 | 92% | 90% |
| ARG1..ng.ml. | ATAD3B..ng.ml. | 0.91 | 92% | 90% |
| ARG1..ng.ml. | DCD..ug.ml. | 0.90 | 92% | 88% |
| IGFALS..ng.ml. | CA2..ng.ml. | 0.91 | 92% | 89% |
| ATAD3B..ng.ml. | CA2..ng.ml. | 0.92 | 92% | 92% |
| IGFALS..ng.ml. | DCD..ug.ml. | 0.90 | 92% | 88% |
| ARG1..ng.ml. | IGFALS..ng.ml. | 0.90 | 92% | 88% |
| ARG1..ng.ml. | LBP..ng.ml. | 0.91 | 92% | 90% |
| LEPTINE..ng.ml. | ARG1..ng.ml. | 0.91 | 92% | 90% |
| IGFALS..ng.ml. | CA2..ng.ml. | 0.91 | 91% | 91% |
| IGFALS..ng.ml. | CA2..ng.ml. | 0.90 | 91% | 89% |
| DCD..ug.ml. | CA2..ng.ml. | 0.92 | 91% | 92% |
| ARG1..ng.ml. | JUP..ng.ml. | 0.90 | 91% | 90% |
| ARG1..ng.ml. | STAT3..ng.ml. | 0.91 | 91% | 91% |
| ARG1..ng.ml. | CA2..ng.ml. | 0.90 | 91% | 90% |
| MAN2A1.ng.ml | CA2..ng.ml. | 0.90 | 91% | 90% |
| CA2..ng.ml. | EGFR..ng.ml. | 0.92 | 90% | 94% |
| DCD..ug.ml. | CA2..ng.ml. | 0.90 | 90% | 90% |
| S100A12..pg.ml. | ARG1..ng.ml. | 0.90 | 90% | 90% |
| IGFALS..ng.ml. | CA2..ng.ml. | 0.91 | 89% | 92% |
| MAN2A1.ng.ml | CA2..ng.ml. | 0.91 | 89% | 92% |
| ARG1..ng.ml. | CA2..ng.ml. | 0.90 | 89% | 91% |
| MAN2A1.ng.ml | CA2..ng.ml. | 0.90 | 89% | 91% |
| DCD..ug.ml. | CA2..ng.ml. | 0.92 | 89% | 94% |
| ATAD3B..ng.ml. | CA2..ng.ml. | 0.91 | 89% | 93% |
| IGFALS..ng.ml. | CA2..ng.ml. | 0.91 | 89% | 92% |
| LEPTINE..ng.ml. | ARG1..ng.ml. | 0.90 | 89% | 91% |
| DCD..ug.ml. | CA2..ng.ml. | 0.90 | 89% | 91% |
| ARG1..ng.ml. | CA2..ng.ml. | 0.90 | 89% | 91% |
| ARG1..ng.ml. | IGFALS..ng.ml. | 0.91 | 89% | 93% |
| KIF20B | ARG1..ng.ml. | 0.91 | 88% | 93% |
| CA2..ng.ml. | STAT3..ng.ml. | 0.90 | 88% | 92% |
| DCD..ug.ml. | CA2..ng.ml. | 0.91 | 88% | 94% |
| DCD..ug.ml. | CA2..ng.ml. | 0.90 | 88% | 92% |
| MAN2A1.ng.ml | CA2..ng.ml. | 0.90 | 88% | 92% |
| ARG1..ng.ml. | DCD..ug.ml. | 0.91 | 88% | 93% |
| ATAD3B..ng.ml. | CA2..ng.ml. | 0.91 | 88% | 93% |
| ATAD3B..ng.ml. | CA2..ng.ml. | 0.91 | 88% | 93% |
| IGFALS..ng.ml. | ATAD3B..ng.ml. | 0.90 | 88% | 93% |
| ARG1..ng.ml. | ATAD3B..ng.ml. | 0.90 | 88% | 93% |
| CA2..ng.ml. | EGFR..ng.ml. | 0.90 | 88% | 92% |
| ARG1..ng.ml. | EGFR..ng.ml. | 0.90 | 88% | 93% |
| ATAD3B..ng.ml. | JUP..ng.ml. | 0.90 | 88% | 93% |
| CA2..ng.ml. | LBP..ng.ml. | 0.91 | 87% | 94% |
| DCD..ug.ml. | CA2..ng.ml. | 0.91 | 87% | 94% |
| KRT19..ng.ml. | ARG1..ng.ml. | 0.91 | 87% | 95% |
| EGFR..ng.ml. | LBP..ng.ml. | 0.90 | 87% | 93% |
| CA2..ng.ml. | STAT3..ng.ml. | 0.90 | 87% | 94% |
| CA2..ng.ml. | STAT3..ng.ml. | 0.90 | 87% | 94% |
| CA2..ng.ml. | STAT3..ng.ml. | 0.90 | 87% | 94% |
| DCD..ug.ml. | CA2..ng.ml. | 0.90 | 87% | 94% |

In addition to the 10 biomarkers that compose either SIG-AGP or SIG-GC, 9 additional candidates, i.e., CA2, MAN2A1, ATAD3B, S100A12, USF2, JUP, CPA4, KRT14 and F13A1 are also promising since they are also found in the combinations of 4 to 6 biomarkers giving the 10 best AUC to predict either preneoplasia or GC either considering ELISA data for CA2, MAN2A1, ATAD3B, S100A12, USF2, JUP or mass spectrometry data for CPA4, KRT14 and F13A1. A summary for this is provided in Table 12 below.

TABLE 12
Summary of promising candidates for incorporation into signatures.

| | | | | | | | Further |
|---|---|---|---|---|---|---|---|
| | | | | | | | promising |
| | Dosage | | | | | | candidates |
| | ELISA | Table | | Table | | | based on |
| Bio- | (Fig. | 8 | SIG- | 10 | SIG- | | ELISA |
| marker | 13) | AG/P | AGP | GC | GC | Table 3 | or MS |
| ARG1 | X | | | X | X | GC | |
| CA2 | X | X | | X | | AGP | ELISA |
| | | | | | | and GC | |
| EGFR | X | X | X | X | | AGP | |
| HP | X | X | X | X | | — | |
| IGFALS | X | X | X | X | | AGP | |
| IL-17 | X | X | | X | X | GC | |
| JUP | X | X | | X | | — | ELISA |
| KRT19 | X | X | X | X | | AGP | |
| LEP | X | X | X | X | X | AGP | |
| | | | | | | and GC | |
| MAN2A1 | X | X | | X | | AGP | ELISA |
| MSLN | X | X | X | X | X | AGP | |
| | | | | | | and GC | |
| SELE | X | X | | | X | AGP | |
| S100A12 | X | X | | X | | — | ELISA |
| TNFalpha | X | | | X | X | GC | |
| USF2 | X | X | | X | | — | ELISA |
| ATAD3B | X | X | | X | | — | ELISA |
| LBP | X | X | | X | | AGP | |
| KIF20B | X | X | | X | | GC | |
| DCD | X | X | | × | | GC | |
| IL-8 | X | X | | X | | | |
| USF1 | X | X | | X | | | |
| STAT3 | X | | | X | | | |
| CPA4 | | | | | | | MS |
| KRT14 | | | | | | | MS |
| F13A1 | | | | | | | MS |

### Conclusion

In conclusion, our analyses led us to shortlist a panel of 40 biomarker candidates described in Table 3 allowing to predict if a patient is affected by either gastritis, gastric pre-neoplasia or cancer lesions. Some candidates give better predictive results than others. Importantly, data from ELISA analysis show that at a first level IL-17 can allow to distinguish with good confidence between healthy subjects and patients. Among the groups of patients, the most striking result indicates that LEP combined to IL-17 can predict GC with high confidence levels and LEP associated with HP or MSLN leads to detect pre-neoplasia (AG/P). Importantly, LEP has been validated by the three approaches developed in this study and IL-17, SELE, MSLN and HP by two of them.

In addition, according to the MS-based analysis carried out, the combination of KIF20B and SPEN could give a good prediction for AG/P or GC. Their combination with KRT19 and S100A12 appears to predict perfectly GC, as summarized in FIG. 12. These promising analyses allow us to propose mathematical models based on combinations of biomarkers to predict the presence of gastric lesions, leading, non limitatively and in a particular embodiment, to a first biomarkers signature to detect the presence of gastric pre-neoplasia and cancer lesions, including IL-17, LEP, HP, MSLN, KIF20B, SPEN, KRT19 and S100A12. KIF20B, SPEN, KRT19 and S100A12 have been identified by the MS proteomic study that included 10 samples per group, further investigation by ELISA assays on all plasma samples of the cohort will help to confirm their accuracy as biomarker, as other potential candidates found in combinations with KIF20B and SPEN listed in Table 6.

According to another aspect, among the proteins initially identified by MS, the plasmatic level measured by ELISA for 12 of them confirmed the MS data leading to consider them as potential biomarkers for the detection of gastric lesions at the different stages of the GC cascade. These 12 proteins in addition to 10 previously selected candidates also quantified by ELISA, led us to define best combinations of 6 biomarkers corresponding, in particular embodiments, to two signatures SIG-AGP and SIG-GC to predict the presence of preneoplasia and GC lesions, respectively. It is to be highlighted that most of the ten candidates included in SIG-AGP and SIG-GC, including MSLN, HP, KRT19, TNFα, IL-17, LEP, ARG1, KIF20B, confirmed the data initially gathered. The predictive score of SIG-GC is excellent. In the case of SIG-AGP the AUC, Sens and Spec are a little bit lower than those obtained with SIG-GC, but still very good. The lowest Spec (79%) observed for SIG-AGP can be due to the heterogeneity of this group of samples which includes atrophic gastritis (AG), intestinal metaplasia (IM) and dysplasia (D). Among this group of patients, all different association of lesions can be found, AG, AG+IM, AG+IM+D. A good prediction for these lesions is extremely important due to the short period of time between the transformation of IM to D, just preceding the development of cancer lesions. It is also these preneoplastic lesions which are the most challenging to identify by endoscopy.

In conclusion, according to this aspect, two biomarker signatures SIG-AGP and SIG-GC were identified and confirmed using three different approaches. These signatures constitute an important tool to predict the presence of gastric preneoplasia and cancer lesions, based on a simple blood sampling and pave the way for future development of a diagnostic non-invasive test to improve the detection/prevention of GC patients. As illustrated in FIG. 16, this test could not only be proposed for a first screening before to drive the patients towards further clinical investigations as endoscopy in the case of positive results, but it could be also useful for the follow-up of patients previously detected with preneoplasia as well as to follow the recurrence/remission of patients under anticancer treatment.

### Perspectives

The characterization of biomarker signatures allowing to predict the presence of NAG, AG/P and GC lesions, paves the way for a future development of a non-invasive prognostic/diagnostic tool for the early detection and prevention of individuals at risk of GC development. This diagnostic test could be, according to a particular embodiment, based on an ELISA assay performed, according to a particular embodiment, on plasma samples, combining the measure of the different factors that compose the biomarker signature allowing to predict the presence of gastric pre-neoplasia and cancer lesions. According to another embodiment, such a diagnostic test could be run using a technology such as a Luminex assay. The concomitant measure of the plasmatic level of each factor that composes this signature and its comparison with the corresponding predetermined cut-off value will indicate the presence or not of pre-neoplasia or cancer lesions. This important tool, based on a simple blood sampling, will allow a first screening of patients at risk of GC leading to drive them toward further clinical investigations. In addition, this diagnostic tool will be also very helpful to predict disease recurrence/outcome and to monitor a personalized treatment and follow-up of patients.

### REFERENCES

1. Bray F, Ferlay J, Soerjomataram I et al. Global cancer statistics 2018: GLOBOCAN estimates of incidence and mortality worldwide for 36 cancers in 185 countries. CA: a cancer journal for clinicians. 2018; 68:394-424.
2. Arnold M, Young Park J, Camargo M C et al. Is gastric cancer becoming a rare disease? A global assessment of predicted incidence trends to 2035. Gut. 2020; 69: 823-829.
3. Correa P. Human gastric carcinogenesis: a multistep and multifactorial process. First American Cancer Society Award Lecture on Cancer Epidemiology and Prevention. Cancer Res. 1992; 52:6735-40.
4. Rugge M, Genta RM, Di Mario F et al. Gastric Cancer as Preventable Disease. Clin Gast Hep: the official clinical practice journal of the American Gastroenterological Association. 2017; 15:1833-43.
5. Ford A C, Forman D, Hunt R H et al. Helicobacter pylori eradication therapy to prevent gastric cancer in healthy asymptomatic infected individuals: systematic review and meta-analysis of randomised controlled trials. BMJ (Clinical research ed). 2014; 348:g3174.
6. Fernandes J, Michel V, Camorlinga-Ponce M et al. Circulating mitochondrial DNA level, a noninvasive biomarker for the early detection of gastric cancer. Cancer Epidemiol Biomarkers Prey. 2014; 23:2430-8.
7. Fan A X, Radpour R, Haghighi M M et al. Mitochondrial DNA content in paired normal and cancerous breast tissue samples from patients with breast cancer. J Cancer Res Clin Oncol. 2009; 135:983-9.
8. Rappsilber J, Mann M, Ishihama Y. Protocol for micro-purification, enrichment, pre-fractionation and storage of peptides for proteomics using StageTips. Nature Prot. 2007; 2:1896-906.
9. Kulak N A, Pichler G, Paron I et al. Minimal, encapsulated proteomic-sample processing applied to copy-number estimation in eukaryotic cells. Nature Meth. 2014; 11:319-24.
10. Cox J, Mann M. MaxQuant enables high peptide identification rates, individualized p.p.b.-range mass accuracies and proteome-wide protein quantification. Nature Biotech. 2008; 26:1367-72.
11. Cox J, Neuhauser N, Michalski A et al. Andromeda: a peptide search engine integrated into the MaxQuant environment. J Prot Res. 2011; 10(:1794-805.
12. Perez-Riverol Y, Csordas A, Bai J et al. The PRIDE database and related tools and resources in 2019: improving support for quantification data. Nucleic Acids Res. 2019; 47:D442-d50.
13. Gorouhi F, Islami F, Bahrami H et al. Tumour-necrosis factor-A polymorphisms and gastric cancer risk: a meta-analysis. Br J Cancer. 2008; 98:1443-51.
14. Corre S, M D Galibert. Upstream stimulating factors: highly versatile stress-responsive transcription factors. Pigment Cell Res 2005; 18:337-48.
15. Chen N, Szentirmay M N, Pawar S A et al. Tumor-suppression function of transcription factor Usf2 in prostate carcinogenesis. Oncogene. 2006; 25:579-87.
16. Costa L, Corre S, Michel Vet al. USF1 defect drives p53 degradation during Helicobacter pylori infection and accelerates gastric carcinogenesis. Gut. 2019.
17. Lu J, Wang Y, Yan M et al. High serum haptoglobin level is associated with tumor progression and predicts poor prognosis in non-small cell lung cancer. Oncotarget. 2016; 7:41758-66.
18. Sun L, Hu S, Yu L et al. Serum haptoglobin as a novel molecular biomarker predicting colorectal cancer hepatic metastasis. Int J Cancer. 2016; 138:2724-31.
19. Jeong S, Oh M J, Kim U et al. Glycosylation of serum haptoglobin as a marker for gastric cancer: an overview for clinicians. Exp. Rev Prot. 2020. 17: 109-117.
20. Endo H, Hosono K, Uchiyama T et al. Leptin acts as a growth factor for colorectal tumours at stages subsequent to tumour initiation in murine colon carcinogenesis. Gut. 2011; 60 :1363-71.
21. Capelle L G, de Vries A C, Haringsma J et al. Serum levels of leptin as marker for patients at high risk of gastric cancer. Helicobacter. 2009. 14: 596-604.
22. Tas F, Karabulut S, Erturk K et al. Clinical significancer of serum leptin levels in patients with gastric cancer. Eur Cytokines Netw. 2018. 29: 52-58.
23. Ho M, Bera T K, Willingham M C et al. Mesothelin expression in human lung cancer. Clin Cancer Res. 2007; 13:1571-5.
24. Meher S, Mishra T S, Sasmal P K et al. Role of biomarkers in diagnosis and prognostic evaluation of acute pancreatitis. J. Biomarkers. 2015. 2015: 519534.
25. Nolen B M, Lokshin A E. Protein biomarkers of ovarian cancer: the forest and the trees. Future Oncol. 2012. 8: 55-71.
26. Cen D, Chen J, Li Z et al. Prognostic significance of cytokeratin 19 expression in pancreatic neuroendocrine tumor: A meta-analysis. PLoS One. 2017; 12:e0187588.
27. Takano M, Shimada K, Fujii T et al. Keratin 19 as a key molecule in progression of human hepatocellular carcinomas through invasion and angiogenesis. BMC Cancer. 2016; 16:903.
28. Liu X, Li Y, Zhang X et al. Inhibition of kinesin family member 20B sensitizes hepatocellular carcinoma cell to microtubule-targeting agents by blocking cytokinesis. Cancer Sci. 2018; 109:3450-60.
29. Legare S, Cavallone L, Mamo A et al. The Estrogen Receptor Cofactor SPEN Functions as a Tumor Suppressor and Candidate Biomarker of Drug Responsiveness in Hormone-Dependent Breast Cancers. Cancer Res. 2015; 75:4351-63.
30. Jang T J, Kim A S, Kim K M. Increased number of arginase-1 positive cells in the stroma of carcinomas compared to precursor lesions and nonneoplastic tissues. Pathology-Research and Practice. 2018. 214:1179-1184.
31. Chen Y, Yang L, Qin Y et al. Effects of differential distributed-JUP on the malignancy of gastric cancer. J. Adv. Res. 2021. 28:195-208
32. Stewart G D, Skipworth R J, Ross J A et al. The dermcidin gene in cancer: role in cachexia, carcinogenesis and tumour cell survival. Curr Opin Clin Nutr Metab Care. 2008. 11:208-13.
33. Merle N, Féraud O, Gilquin B et al. ATAD3B is a human embryonic stem cell specific mitochondrial protein, re-expressed in cancer cells, that functions as dominant negative for the ubiquitous ATAD3A. Mitochondrion. 2012. 12: 441-448.
34. Parkkila S, Lasota J, Fletcher J A et al. Carbonic anhydrase II. A novel biomarker for gastrointestinal stromal tumors. Modern Pathology. 2010. 23: 743-750
35. Ozcan S, Bark D A, Ruhak Lr et al. Serum glycan signatures of gastric cancer. Cancer Prey Res. 2014. 7: 226-235.
36. Marquardt J U, Seo D, Andersen J B et al. Sequential transcriptome analysis of human liver cancer indicates late stage acquisition of malignant traits. J Hepatol. 2014. 60:346-353.
37. Ashrafizadeh M, Zarrabi A, Orouei S et al. Stat3 pathways in gastric cancer: signaling, therapeutic targeting and future prospects. Biology (Basel). 2020. 9:126.
38. Liu H, Du T, Yang G. STAT3 phosphorylation in central leptin resistance. Nutrition and Metabolism. 2021. 18 : 39.
39. Lieto E, Ferraraccio F, Orditura M et al. Expression of vascular endothelial growth factor (VEGF) and epidermal growth factor (EGFR) is an indicator of independent prognosis of worse outcome in gastric cancer patients. 2008. Ann Surg Oncol. 15: 69-79.

1. An in vitro method of determining whether a human patient has lesions rendering said patient at risk of a gastric cancer condition and/or needs further medical test in relation thereto, comprising screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, said method comprising the steps of:
a. determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level with the proviso that the selected biomarkers do not consist of the association of IL-8 and mtDNA level, optionally determining the level of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level,
b. comparing the levels determined in step a. to a control, and
c. if levels of at least two biomarkers as determined and compared in steps a. and b. deviate from the levels of their controls, conclusion is made that the human patient has lesions rendering said patient at risk of a gastric cancer condition, and/or needs further medical test in relation thereto, especially clinical investigation.

2. The method of claim 1, wherein step a. consists of determining the level of at least three biomarkers, encompassing the two biomarkers that are IL-8 protein and mtDNA level, in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE and MSLN, optionally encompassing the two biomarkers that are IL-8 protein and mtDNA level in further combination with one or more biomarker(s) selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-17, TNF-alpha, USF1, USF2, SELE, EGFR, STAT3 and MSLN.

3. The method of claim 1, wherein step a. consists of determining the level of at least three biomarkers, including two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, and mtDNA level, and at least one further biomarker selected amongst: EGFR and STAT3.

4. The method of claim 1, wherein step a. consists of determining the level of at least three biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level.

5. The method of claim 1, wherein step a. consists of determining the level of at least two, preferably between two and six, biomarkers selected amongst: IGFALS, KRT19, CPA4, CA2, MAN2A1, KIF20B, JUP, F13A1, LBP, KRT14, ARG1, S100A12, ATAD3B, DCD, HP, LEP, IL-8, IL-17, TNF-alpha, USF1, USF2, SELE, MSLN, EGFR and STAT3.

6. (canceled)

7. (canceled)

8. (canceled)

9. The method according to claim 1, which is for assessing the risk that a human patient has an atrophic gastritis/pre-neoplasia (AG/P) with a sensitivity of at least 80% and/or a specificity of at least 80%, in particular a sensitivity and a specificity of at least 80% each.

10. (canceled)

11. (canceled)

12. The method according to claim 1, , which is for assessing the risk that a human patient has gastric cancer (GC) with a sensitivity of at least 75% and/or a specificity of at least 90%, in particular a sensitivity and a specificity of at least 90% each.

13. (canceled)

14. The method of claim 1, which is for monitoring or diagnosing the health status of a patient susceptible of suffering from condition(s) susceptible to evolve in a gastric cancer condition or susceptible of suffering from a gastric pre-cancer condition or susceptible of suffering from a gastric cancer condition, or the health status of a human patient that has lesions rendering said patient at risk of a gastric cancer condition, wherein the method is repeated at least once over time so as to conclude about the health status of the tested patient if the comparison set in step b. of claim 1 and/or the deviation observed in step c. of claim 1 shows an evolution, in particular for monitoring or diagnosing the health status of a patient diagnosed with gastric cancer, and optionally treated for gastric cancer.

15. The method of claim 1, which comprises as a distinct, simultaneous or parallel step, a step of detecting an Helicobacter pylori infection, in particular through detection of antigen(s) specific for H. pylori infection, or through an assay involving DNA amplification and subsequent detection of said DNA, or detection of the presence of specific H. pylori IgA and IgG antibodies in a biological sample previously removed from the tested patient, or through an 13C urea breath test performed on the tested patient.

16. The method of claim 1, wherein the blood sample is from a patient diagnosed with gastric carcinogenesis and optionally said patient is under treatment for this condition, and/or is from a patient having an ongoing, treated or not, Helicobacter pylori infection, and/or a patient having antecedents of Helicobacter pylori infection(s), eradicated or not, and/or an individual having gastric pain and/or a family history of gastric cancer.

17. The method of claim 1, wherein the levels of plasmatic biomarkers are determined by enzyme-linked immunosorbent assay (ELISA) testing, or Mass Spectrometry, or quantitative polymerase chain reaction (q-PCR), or Luminex assay, and, when carried out, the level of mtDNA is determined by quantitative polymerase chain reaction (q-PCR).

18. Kit suitable for carrying out a method as defined in claim 1, comprising:
at least two types of antibodies having different antigen specificity wherein each type of antibody is specific for a protein selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins or a combination of several antibodies having different antigen specificity for PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3, and MSLN proteins, and, optionally, at least an antibody specific for H. pylori antigen(s), such as CagA antigens, and, optionally one or several of the following reagents,
a secondary antibody, such as a biotinylated antibody, or reagent to reveal a complex between specific antibody(ies) recited above and its(their) target,
optionally, beads such as color-coded beads, and/or magnetic or non-magnetic beads, and/or carboxylated beads, with optionally an amine coupling kit for attaching antibodies to beads,
optionally, phycoerythrin (PE)-conjugated streptavidin to reveal biotinylated antibodies,
optionally, a buffer solution,
optionally, an assay plate, and
optionally a notice providing instructions for use and expected values for interpretation of results.

19. Kit suitable for carrying out a method as defined in claim 1, comprising:
At least one pair of specific oligonucleotide primers specific for hybridization with mtDNA, and/or
At least two pairs of specific oligonucleotide primers specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins, and, optionally, at least one pair of specific oligonucleotide primers or nucleic acid molecule specific for hybridization with H. pylori nucleic acid(s) sequence(s), and, optionally, one or several of the following reagents,
nucleotides (e.g. dATP, dCTP, dGTP, dUTP),
a DNA polymerase, in particular a thermostable DNA polymerase, such as a Taq DNA Polymerase,
at least one dye for staining nucleic acids, in particular a dye detectable in a real-time PCT equipment,
optionally, a buffer solution,
optionally, reagents necessary for the hybridization of the primers to their targets,
optionally, a reference dye and,
a notice providing instructions for use and expected values for interpretation of results.

20. Kit suitable for carrying out a method as defined in claim 1, comprising:
at least two types of antibodies having different antigen specificity wherein each type of antibody is specific for a protein selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins or a combination of several antibodies having different antigen specificity for PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3, MSLN proteins, and, optionally, at least an antibody specific for H. pylori antigen(s), such as CagA antigens, and, optionally one or several of the following reagents,
a secondary antibody, such as a biotinylated antibody, or reagent to reveal a complex between specific antibody(ies) recited above and its(their) target,
optionally, beads such as color-coded beads, and/or magnetic or non-magnetic beads, and/or carboxylated beads, with optionally an amine coupling kit for attaching antibodies to beads,
optionally, phycoerythrin (PE)-conjugated streptavidin to reveal biotinylated antibodies,
optionally, a buffer solution,
optionally, an assay plate, and
optionally a notice providing instructions for use and expected values for interpretation of results, and
At least one pair of specific oligonucleotide primers specific for hybridization with mtDNA and/or
At least two pairs of specific oligonucleotide primers specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and, optionally, at least one pair of specific oligonucleotide primers or nucleic acid molecule specific for hybridization with H. pylori nucleic acid(s) sequence(s), and, optionally, one or several of the following reagents,
nucleotides (e.g. dATP, dCTP, dGTP, dUTP),
a DNA polymerase, in particular a thermostable DNA polymerase, such as a Taq DNA Polymerase,
at least one dye for staining nucleic acids, in particular a dye detectable in a real-time PCT equipment,
optionally, at least one buffer solution,
optionally, reagents necessary for the hybridization of the primers to their targets,
optionally, a reference dye.

21. Set of markers comprising or consisting of at least two antibodies specific for a protein selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and optionally at least one pair of specific oligonucleotide primers, or nucleic acid molecules, specific for hybridization with mtDNA, or set of markers comprising or consisting of at least two pairs of specific oligonucleotide primers specific for hybridization with the DNA regions coding for, respectively, two or more of PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, EGFR, STAT3 and MSLN proteins and optionally at least one pair of specific oligonucleotide primers or nucleic acid molecule(s) specific for hybridization with mtDNA, suitable to carry out a method as defined in claim 1.

22. Method for using the set of markers according to claim 21 for determining whether a human patient has lesions rendering said patient at risk of a gastric cancer condition and/or needs further medical test in relation thereto, or for prognosing or diagnosing a gastric pre-cancer condition or a gastric cancer condition, by screening a biological sample of blood or plasma previously removed from a human patient susceptible of suffering of condition(s) susceptible to evolve in a gastric cancer condition or susceptible of suffering from a gastric pre-cancer condition or susceptible of suffering from a gastric cancer condition, by measuring the level of the at least two markers in a biological blood or plasma sample removed from a human patient susceptible of suffering of condition(s) susceptible to evolve in a gastric cancer condition or susceptible of suffering from a gastric pre-cancer condition or susceptible of suffering from a gastric cancer condition.

23. A method carried out by a computer for investigating whether a human patient has lesions rendering said patient at risk of a gastric cancer condition and/or needs further medical test in relation thereto, the method comprising the steps of:
a. receiving the levels of at least two biomarkers selected amongst: PGK1, CFP, IGFALS, KRT19, SPRR1A, CPA4, CA2, SERPINA5, MAN2A1, KIF20B, SPEN, JUP, KRT6C, CDSN, KPRP, F13A1, SAA1(SAA2), LBP, DSP, KRT2, KRT14, ARG1, S100A12, ATAD3B, MAN1A1, HAL, DCD, C7, HP, LEP, IL-8, IL-17, TNF-alpha, USF1, USF2, SELE, MSLN, EGFR, STAT3 and mtDNA level with the proviso that the selected biomarkers do not consist of the association of IL-8 and mtDNA level, or receiving any levels of at least two biomarkers whose determination is defined in claim 1, in particular where the levels of the at least two biomarkers are measured through an in vitro method for measuring such levels, or a device adapted to the same, and
b. processing the levels determined in step a. by comparing them to a control, and
c. determining through a predetermined decision rule, especially a decision rule associated with determined sensitivity and/or specificity associated with the investigated biomarkers, in particular if the levels of at least two biomarkers compared in step b. deviate from their controls, whether the levels received in step a. make that the human patient from which they have been measured, especially through an in vitro method for measuring the levels defined in step a., has lesions rendering said patient at risk of a gastric cancer condition, and/or needs further medical test in relation thereto, especially clinical investigation.

24. (canceled)

25. The data processing apparatus of claim 24, which comprises:
an input interface to receive the levels of the at least two biomarkers,
a memory for storing at least instructions of a computer program comprising instructions which, when the program is executed by a computer or processor, cause the computer to carry out the method of claim 23, optionally a memory for storing control data and decision rules,
a processor accessing to the memory for reading the aforesaid instructions and executing the method,
an output interface to provide at least the determination of whether the levels of the at least two biomarkers make that the human patient from which they have been measured has lesions rendering said patient at risk of a gastric cancer condition, and/or needs further medical test in relation thereto, especially clinical investigation.

26. A computer program comprising instructions which, when the program is executed by a computer or processor, cause the computer to carry out the method of claim 23.

27. A computer-readable medium, in particular a computer-readable non-transient recording medium, having stored thereon the computer program of claim 26.
